(19) United States
(12) Patent Application Publication
(10) Pub. No.: US 2017/0247769 A1
(43) Pub. Date: Aug. 31, 2017

(54) BIOSENSORS AND METHODS OF USE

(71) Applicants: CARNEGIE INSTITUTION OF WASHINGTON, WASHINGTON, DC (US); THE BOARD OF TRUSTEES OF THE LELAND STANFORD JUNIOR UNIVERSITY, STANFORD, CA (US)

(72) Inventors: Cindy AST, Stanford, CA (US); Luke M. OLTROGGE, Stanford, CA (US); Wolf B. FROMMER, Stanford, CA (US)

(21) Appl. No.: 15/438,078

(22) Filed: Feb. 21, 2017

Related U.S. Application Data
(60) Provisional application No. 62/298,211, filed on Feb. 22, 2016.

(51) Int. Cl. C12Q 1/68; C07K 14/435

(52) U.S. Cl. CPC C12Q 1/6897; C07K 14/43595; C07K 2319/60

(57) ABSTRACT

The present disclosure provides fluorescent polypeptides containing a fusion of a circularly permuted, first fluorescent protein and a second fluorescent protein, the first fluorescent protein containing a first fluorescent moiety and the second fluorescent protein containg a second fluorescent moiety, wherein the second fluorescent protein is contained in the circularly permuted first fluorescent protein to form a cassette that can be inserted into sites within a sensing protein of interest to form novel biosensors; alternatively, the reference domain can be inserted into an existing single-fluorescent protein-based, intensiometric biosensor in order to make a ratiometric biosensor in one cloning step; nucleic acid sequences encoding the fluorescent polypeptides, fluorescent sensors including the fluorescent polypeptides, and methods of making and using same are described herein.

The present application claims benefit of U.S. Provisional Patent Application No. 62/298,211, filed Feb. 22, 2016, the entire contents of which is incorporated herein by reference.

Current biosensors with the highest dynamic range are intensiometric (based on the read-out of a single wavelength) and deploy environmentally-sensitive circularly permuted fluorescent proteins (cpFPs). These intensiometric biosensors have the drawback, however, of only allowing relative quantitation. Ratiometric biosensors arerobust to intensity changes due to changes in variable expression levels and/or instrument-related artefacts (changes in laser intensity, focus etc) and thus facilitate absolute analyte quantification. Unfortunately most suffer from poor dynamic ranges. To enable ratiometric analyses while retaining the high dynamic range, biosensors containing nested fluorescent proteins (generally referred to herein as “Matryoshka” biosensors due to their resemblance to the nested Russian doll) have been engineered and are described herein.

The biosensors of the presently disclosed technology are exemplified herein by incorporation of a fluorescent polypeptide of the present disclosure. Fluorescent polypeptides of the present disclosure are exemplified by a fusion of a circularly permuted superfolder green FP (cpsfGFP) (first protein) and a large Stokes shift orange fluorescent protein (LSSmOrange) (second protein) wherein the second protein is contained in the first protein. An exemplified fluorescent polypeptide of the presently disclosed technology containing cpsfGFP and LSSmOrange is referred to herein as GO-Matryoshka (wherein “GO” refers to “green-orange”). At 442 nm excitation, GO-Matryoshka shows distinct green and orange emission bands, while 488 nm excitation leads to green emission alone. The laser lines 442 and 488 are common fluorescence excitation sources. GO-Matryoshka is demonstrated herein to be useful in biosensors of the present disclosure as a replacement for cpEGFP in ultrasensitive calcium sensors, such as GCaMP6s, and the ammonium transporter activity sensor AmTrac, thereby endowing these sensors with ratiometric outputs for absolute quantitation.

The presently disclosed technology includes therefore fluorescent sensors or biosensors, such as are exemplified by the calcium and ammonium transporter activity sensors described herein (generally referred to as “MatryoshCaMP” and “AmTryoshka”, respectively herein), containing fluorescent polypeptides of the present disclosure.

Biosensors of the present disclosure combine the advantages of intensiometric and ratiometric FRET (Förster Resonance Energy Transfer)-based biosensors.

The development of fluorescent protein (FP)-based, genetically-encoded biosensors of the present disclosure provides realtime monitoring of dynamic biological processes such as ion signaling, hormone dynamics, metabolism, protein transport and receptor activation with high spatial and temporal resolution in living cells or organisms¹.

Depending on the fluorescence read-out, biosensors can generally be divided into two major categories: single-FP or ratiometric, dual-FP sensors.

Single-FP sensors are comprised of one FP, either exploiting the intrinsic sensitivities of FPs alone towards certain stimuli, such as pH², or by connecting the sensing domain to circularly permuted FPs (cpFPs)³. Structural changes of the sensor domain due to target binding or signal perception can be transferred to the cpFP and visualized as a change in the fluorescence intensity (FI). Numerous biosensors of different hues exploit this design principle, including a palette of calcium sensors³⁻⁸ with recent additions of photoconvertible variants^{9,10}, sensors for metabolites, such as maltose, and protein transporter activity¹².

Single-FP-based biosensors may show a large dynamic range but most are intensiometric, as they rely on the readout of a single fluorescence intensity and thus do not provide ratiometric information. However, absolute concentration knowledge is crucial for accurate analyte observations as potential inconsistencies in expression level as well as instrumental artifacts may occur, particularly during long-term experiments. To address this, a few ratiometric single-FP biosensors were developed, displaying two excitation or emission maxima with opposite intensity changes^{7,13,14}. These systems make for difficult sensor design because the protein has many additional constraints. The excited-state proton transfer (ESPT) network must remain intact and the fluorescence quantum yield of the protonated species must be preserved while trying to optimize the dynamic range.

Alternatively, a spectrally distinct FP has been co-expressed as reference^{8,15-17}. Most previously available ratiometric biosensors (FIG. 1) consist of a target-sensing domain sandwiched between two FPs (such as FP1 and FP2 of FIG. 1) and usually exploit FRET, i.e. the distance-dependent, non-radiative energy transfer between a donor and an acceptor FP^{1,18,19}. A conformational change of the sensor domain upon target perception alters the efficiency of FRET. The acceptor FP has been used as control as it can be directly excited and monitored. However, FRET-based biosensor are restricted in their dynamic range due to the large size of the FP barrel limiting the distance of the chromophores²⁰ and potential rotational averaging due to fluctuations in FP dipole orientations²¹.

The presently disclosed technology provides a biosensor design (FIG. 3) which combines the advantages of single- and FRET-based sensors, i.e. high FI (fluorescent intensity), large dynamic range and ratiometric read-out into one fluorescent polypeptide entity (FIG. 2) that can be readily employed for novel sensor construction. Furthermore the modular nature of this technology can be readily used to upgrade existing intensiometric single-FP biosensors to be ratiometric. To maintain the large dynamic range demonstrated by cpFP-based sensors, circularly permuted superfolder GFP (cpsfGFP)²²⁻²⁴ has been used in the exemplified fluorescent polypeptides and biosensors as the cpsfGFP is fast-maturing, particularly stable and tolerant of insertions. In an exemplified fluorescence polypeptide a large Stokes shift (LSS) mOrange^{25,26} was inserted between the native N- and C-termini of cpsfGFP. This FP-fusion showed no detectable changes in the photophysical properties with regard to steady-state analysis compared to the individual FPs cpsfGFP and LSSmOrange. The exemplified FP-fusion is excited at a single wavelength of λ_{exc}˜442 nm leading to two emission bands at λₑₘ˜510 nm and λₑₘ˜570 nm. Green emission (λₑₘ˜510 nm) with only minimal cross-excitation of the LSSmOrange was observed with excitation at λ_{exc}˜488 nm.

The presently disclosed technology however is not limited to combinations of cpsfGFP and LSSmOrange but is broadly applicable to combinations of circularly permuted fluorescent proteins, fluorescent proteins generally and biosensors containing fluorescent polypeptides of the disclosure generally.

Insertion of GO-Matryoshka or variations into a suitable position of sensor domain, such as schematically depicted in FIG. 3, allows the creation of a ratiometric biosensor of the presently disclosed technology in a single step.

Ratiometric calcium sensors based on GCaMP6s⁸ of the presently disclosed technology are described herein. GCaMPs are the most widely applied calcium sensors and have undergone multiple rounds of structure-guided optimization. GCaMP6s are comprised of a cpEGFP centered between the calcium-binding protein calmodulin (CaM) and CaM-interacting M13 peptide (FIG. 4). For normalization of resting FI, a red-shifted FP, such as mCherry, is generally co-expressed, e.g. in the nucleus⁸. However, this can be a problematic normalization procedure since there is uncertainty in the expression levels of both the sensor and the mCherry.

An exemplified embodiment of a calcium biosensor of the present disclosure is depicted in FIG. 5 wherein the fluorescent polypeptide may be a GO-Matryoshka of the present disclosure and wherein the circularly permuted fluorescent protein of the GO-Matryoshka may be either the cpEGFP of GCaMP6s or a cpsfGFP as described herein.

Further detailed herein are the effects of residue histidine (78H) that was reported to yield the most sensitive calcium response in the GCaMP6 set screen. The in vitro characterization and evaluation of both excitation wavelengths (442 nm and 488 nm), of the different MatryoshCaMP variants exemplified herein (i.e., wherein the circularly permuted fluorescent protein is either cpEGFP or a cpsfGFP) revealed no detectable modifications of the sensors properties due to the presence of the LSSmOrange when compared to the cpFP-based GCaMP controls without the second fluorescent protein of the presently disclosed technology.

The cpEGFP-based MatryoshCaMP, cpsfGFP-based sfMatryoshCaMP and sfMatryoshCaMP-T78H demonstrated different calcium binding affinities, sensitivities and chromophore pKₐ values due to different characteristics of the cpEGFP or cpsfGFP. Depending on the application, each MatryoshCaMP version can be individually beneficial.

As a further exemplification of the presently disclosed technology, a GO-Matryoshka of the present disclosure was used as a part of an AmTrac biosensor wherein an existing cpEGFP in the ammonium activity state sensor AmTrac was replaced with a GO-Matryoshka. AmTrac is based on a cpEGFP introduced into the Arabidopsis thaliana Ammonium Transporter 1;3 (AtAMT1;3)¹² (FIG. 6). AmTrac is intensiometric and attempts to render AmTrac ratiometric by attaching a second FP have been unsuccessful.

The exemplified construct of the present disclosure based on AmTrac, termed AmTryoshka, was tested in living yeast cells. Initially, AmTryoshka did not show a response towards saturating ammonium conditions, most likely due to inhibited ammonium transport. Identification of two individual mutations restored the transport phenotype. The fluorescent intensity (FI) changed up to 30% in the green emission channel as response to ammonium. The LSSmOrange served as non-responsive control.

The presently disclosed technology therefore is broadly applicable in that the fluorescent polypeptides of the present disclosure may be used to generate ratiometric biosensors with large dynamic range, such as in the high-performance calcium and ammonium transport activity sensors exemplified herein. Different fluorescent protein combinations, such as are described herein, may be used in known insertion sites or variations of insertion sites of known sensors. This will be especially useful in, for example, sensitive proteins, such as transporters, where only one insertion position may be tolerated. In a similar manner, the second fluorescent protein of the present disclosure may be inserted in the sequence regions between the β-strands of the first fluorescent protein of the present disclosure which may be generally identified in a manner known in the art. See for example, Tsien (U.S. Pat. No. 7,060,793), Waldo et al. (U.S. Patent Application Publication No. 2015/0099271 and U.S. Pat. No. 7,955,821), Frommer et al. (U.S. Pat. No. 9,176,143 and U.S. Patent Application Publication No. 2014/0356896), and Pedelacq et al (“Engineering and characterization of a superfolder green fluorescent protein”, Nature Biotechnology, volume 24, number 1, January 2006) (the entire contents of each of which is hereby incorporated herein by reference).

### BRIEF DESCRIPTION OF DRAWINGS

FIG. 1 is a schematic of ratiometric biosensors of the prior art consisting of a target-sensing domain sandwiched between two FPs which usually exploit FRET (Förster Resonance Energy Transfer).

FIG. 2 is a schematic of a fluorescent polypeptide of the present disclosure wherein (—C) schematically depicts the region of the native C-terminus of the first fluorescent protein and (—N) schematically depicts the region of the native N-terminus of the first fluorescent protein.

FIG. 3 is a schematic of a biosensor of the present disclosure.

FIG. 4 is a schematic of an available calcium sensor GCaMP6s.

FIG. 5 is a schematic of calcium sensor of the present disclosure.

FIG. 6 is a schematic of an AmTrac biosensor as described in U.S. Patent Application Publication No. 2014/0356896 (Frommer et al, published Dec. 4, 2014).

FIG. 7A. Schematic representation of GO-Matryoshka with the LSSmOrange sandwiched between the reversed C- and N-termini of the sfGFP, connected by the optional flexible first and second linker, depicted as GGT and GGS in the schematic and dashed lines in drawing L1 and L2 indicate the left and right peptide linker, which are LS and FN, respectively, in this exemplified embodiment.

FIG. 7B. Steady-state fluorescence excitation (two dashed lines to the left) and emission (two solid lines to the right) of cpsfGFP (λ_{exc} 440 nm, λₑₘ 550 nm; black—two middle traces) and LSSmOrange (λ_{exc} 440 nm, λₑₘ 570 nm; grey—two outer traces).

FIG. 7C. Steady-state fluorescence excitation (λₑₘ 570 nm; dashed line large left trace) and emission (λ_{exc} 440 nm; solid line—two right peaks) of GO-Matryoshka (black-grey). Excitation trace with grey to left and black to right, and emission trace of two emission peaks of black (left) and grey (right).

FIG. 8A. Schematic representation of MatryoshCaMP and sfMatryoshCaMP, with the LSSmOrange inserted between the native C- and N-terminus of the EGFP or sfGFP, sandwiched between the M13 peptide and Calmodulin domain. LE and LP indicate the peptide linker. T78H mutation included in sfMatryoshCaMP-T78H (in sfGFP-C*).

FIG. 8B. Normalized calcium affinity titration of MatryoshCaMP (square, left trace), sfMatryoshCaMP-T78H (circle, middle trace) and sfMatryoshCaMP (triangle, right trace). Data were corrected for fluorescence bleed-through (bleed-through factor 0.10).

FIG. 8C. Steady-state fluorescence spectra (λ_{exc} 440 nm) of calcium titration of MatryoshCaMP, sfMatryoshCaMP-T78H and sfMatryoshCaMP, respectively left to right.

FIG. 9A. Schematic representation of AmTryoshka, with LSSmOrange sandwiched between the native C- and N-termini of the sfGFP and this cassette inserted into loop 5-6 (between transmembrane helix 5-6) of AtAMT1.3. LS and FN indicate the peptide linker.

FIG. 9B-1. AmTryoshka constructs generated from five (5) different orange or red FPs. Five (5) different orange or red fluorescent proteins were tested for Matryoshka approach. sfAmTrac-GS served as basis for insertion of either of the red-shifted FP into the middle of the GGTGGS linker of the cpsfGFP inside the AtAMT1;3. Steady-state spectra were obtained from liquid cultures of yeast triple mutant transformed with the indicated constructs and measured at OD600˜0.5. Normalized fluorescence emission spectra (λexc=480 nm) with sfAmTrac-GS as control.

FIG. 9B-2. Same plot as in FIG. 9B-1 but includes the LSSmOrange variant (λexc=440 nm) to compare the relative orange and red maxima. Note: no shift in the emission peak upon insertion of second FP.

FIG. 9B-3. Same as in FIG. 9B. Plot of absolute intensities (λexc=480 nm). Note: mCherry, mKate2 and Katushka lead to a decrease in overall green intensity when inserted into sfAmTrac-GS. The red fluorescence maxima seen in FIG. 9B-1 are a result of FRET from cpsfGFP to the orange or red FP. The sfGS-LSSmOrange (later termed AmTryoshka-GS) construct differs, since the green and orange emission derives from direct excitation of both FPs at λexc=440 nm (see FIG. 9B-2)

FIG. 9C. Yeast complementation assay of yeast Δmep1,2,3 mutant transformed with indicated constructs and grown on solid media with indicated N-sources. Arginine served as growth control. Vector control served as negative control.

FIG. 9D. Relative fluorescence intensity (normalized to sfAmTrac-LS=1) and fluorescence response in the green channel after addition of 1 mM NH₄Cl (mean±SEM; n=3).

FIG. 9E. Steady-state emission spectra of AmTryoshka-LS-F138I and -T78H with λ_{exc} 440 nm. Treatment with NH₄Cl at indicated concentration. Spectra were normalized to the maximum intensity.

FIG. 9F. Corresponding titration of ΔR/R₀ (R=FI510 nm/F1570 nm) of AmTryoshka-LS-F138I and -T78H (black squares) and the Hill fit (black line). Data were corrected for fluorescence bleed-through (bleed-through factor 0.08) and normalized to water-treated controls.

FIG. 9G. Fluorescence response (ΔR/R₀) of Δmep1,2,3 or wild type (wt) transformed with AmTryoshka-LS-F138I, -T78H or the non-responsive control AmTryoshka-GS (mean±SEM; n=3).

FIG. 9H. Plot of fluorescence change as response towards 1 mM NH₄Cl over FI. Comparison of AmTrac-LE (empty diamond—left most) 12 with the cpsfGFP-based sfAmTracs containing the left linker peptides LE, LS and GS (grey diamond—3 right).

FIG. 9I. AmTroshka LS-F138I, GS-F138I and -L255I. Steady-state emission spectra with λ_{exc} 440 nm after treatment with indicated NH₄Cl concentrations (normalization to highest value) on the left. Corresponding titration of the fluorescent response ΔR/R₀ (R=FI₅₁₀ₙₘ/FI₅₇₀ₙₘ) (black square) and Hill fit (black line) on the right. Data were corrected for bleed-through (bleed-through factor 0.08) and normalized to water-treated controls.

FIG. 9J. Titration of sfAmTrac-LS and -GS with the mutations F138I and L255I. Steady-state emission spectra with λ_{exc} 440 nm after treatment with increasing NH₄Cl concentrations and normalized to water treated control on the left. Corresponding titration of the fluorescence response ΔF/F₀ (F=FI₅₁₀ₙₘ) (black square) and the Hill fit (black line) on the right. Data were normalized to water-treated controls.

FIG. 10. Amino acid and DNA sequences of cpEGFP (SEQ ID NOs:26 and 25, respectively). Chromophore TYG is amino acids 162-164.

FIG. 11. Amino acid and DNA sequences of cpsfGFP (SEQ ID NOs:7 and 6, respectively). Chromophore TYG is amino acids 163-165.

FIG. 12. Amino acid and DNA sequences of cpsfGFP-T78H (SEQ ID NOs:10 and 9, respectively). Mutation at position 21 of amino acid sequence and chromophore TYG is amino acids 163-165.

FIG. 13. Amino acid and DNA sequences of GO-Matryoshka (LS-FN) (SEQ ID NOs:30 and 29, respectively). LS linker (amino acids 1-2), cpsfGFP sequence (amino acids 3-91), GGT linker (amino acids 92-94), LSSmOrange (amino acids 95-330), GGS linker (amino acids 331-333), cpsfGFP sequence (amino acids 334-459), FN linker (amino acids 460-461).

FIG. 14. GO-Matryoshka (LS-FN) T78H amino acid and DNA sequences (SEQ ID NOs:164 and 163, respectively). LS linker (amino acids 1-2), cpsfGFP sequence (amino acids 3-91), T78H (amino acid 20), GGT linker (amino acids 92-94), LSSmOrange (amino acids 95-330), GGS linker (amino acids 331-333), cpsfGFP sequence (amino acids 334-459), FN linker (amino acids 460-461).

FIG. 15. AtAMT1;3 amino acid and DNA sequences (SEQ ID NOs:34 and 165, respectively).

FIGS. 16A and 16B. AmTrac-LE (AmTrac) amino acid and DNA sequences (SEQ ID NOs:167 and 166, respectively). LE linker (amino acids 234-235), cpEGFP (amino acids 236-476) and FN linker (amino acids 477-478).

FIGS. 17A and 17B. deAmTrac-CP amino acid and DNA sequences (SEQ ID NOs:169 and 168, respectively). CP linker (amino acids 234-235), cpEGFP (amino acids 236-476) and FN linker (amino acids 477-478).

FIGS. 18A and 18B. deAmTrac-FP amino acid and DNA sequences (SEQ ID NOs:170 and 171, respectively). FP linker (amino acids 234-235), cpEGFP (amino acids 236-476) and FN linker (amino acids 477-478).

FIGS. 19A and 19B. sfAmTrac-LE amino acid and DNA sequences (SEQ ID NOs:172 and 173, respectively). LE linker (amino acids 234-235), cpsfGFP (amino acids 236-474) and FN linker (amino acids 475-476).

FIGS. 20A and 20B. sfAmTrac-LS amino acid and DNA sequences (SEQ ID NOs:174 and 175, respectively). LS linker (amino acids 234-235), cpsfGFP (amino acids 236-474) and FN linker (amino acids 475-476).

FIGS. 21A and 21B. sfAmTrac-GS amino acid and DNA sequences (SEQ ID NOs:176 and 177, respectively). GS linker (amino acids 234-235), cpsfGFP (amino acids 236-474) and FN linker (amino acids 475-476).

FIGS. 22A and 22B. AmTryoshka-GS amino acid and DNA sequences (SEQ ID NOs:178 and 179, respectively). GS linker (amino acids 234-235), cpsfGFP (amino acids 236-324), GGT linker (amino acids 325-327), LSSmOrange (amino acids 328-563), GGS linker (amino acids 564-566), cpsfGFP (amino acids 567-710) and FN linker (amino acids 711-712).

FIGS. 23A and 23B. AmTryoshka-GS-F138I amino acid and DNA sequences (SEQ ID NOs:180 and 181, respectively). F138I suppressor mutation (amino acid 138 (nucleotide 412)), GS linker (amino acids 234-235), cpsfGFP (amino acids 236-324), GGT linker (amino acids 325-327), LSSmOrange (amino acids 328-563), GGS linker (amino acids 564-566), cpsfGFP (amino acids 567-710) and FN linker (amino acids 711-712).

FIGS. 24A and 24B. AmTryoshka-GS-L255I amino acid and DNA sequences (SEQ ID NOs:182 and 183, respectively). GS linker (amino acids 234-235), cpsfGFP (amino acids 236-324), GGT linker (amino acids 325-327), LSSmOrange (amino acids 328-563), GGS linker (amino acids 564-566), cpsfGFP (amino acids 567-710), FN linker (amino acids 711-712) and L255I suppressor mutation (amino acid 734 (nucleotide 2200)).

FIGS. 25A and 25B. AmTryoshka-LS-F138I amino acid and DNA sequences (SEQ ID NOs:184 and 185, respectively). F138I suppressor mutation (amino acid 138 (nucleotide 412)), LS linker (amino acids 234-235), cpsfGFP (amino acids 236-324), GGT linker (amino acids 325-327), LSSmOrange (amino acids 328-563), GGS linker (amino acids 564-566), cpsfGFP (amino acids 567-710), and FN linker (amino acids 711-712).

FIGS. 26A and 26B. AmTryoshka-LS-L255I amino acid and DNA sequences (SEQ ID NOs:186 and 187, respectively). LS linker (amino acids 234-235), cpsfGFP (amino acids 236-324), GGT linker (amino acids 325-327), LSSmOrange (amino acids 328-563), GGS linker (amino acids 564-566), cpsfGFP (amino acids 567-710), FN linker (amino acids 711-712) and L255I suppressor mutation (amino acid 734 (nucleotide 2200)).

FIG. 27. GCaMP6s amino acid and DNA sequences (SEQ ID NOs:188 and 189, respectively). M13 peptide (amino acids 1-21), LE linker (amino acids 22-23), cpsfGFP (amino acids 24-264), and LP linker (amino acids 265-266).

FIG. 28. sfGaMP amino acid and DNA sequences (SEQ ID NOs:190 and 191, respectively). M13 peptide (amino acids 1-21), LE linker (amino acids 22-23), cpEGFP (amino acids 24-262), and LP linker (amino acids 263-264).

FIG. 29. sfGaMP-T78H amino acid and DNA sequences (SEQ ID NOs:192 and 193, respectively). M13 peptide (amino acids 1-21), LE linker (amino acids 22-23), cpsfGFP (amino acids 24-262), T78H mutation (amino acid 41) and LP linker (amino acids 263-264).

FIGS. 30A and 30B. MatryoshCaMP amino acid and DNA sequences (SEQ ID NOs:194 and 195, respectively). M13 peptide (amino acids 1-21), LE linker (amino acids 22-23), cpEGFP (amino acids 24-113), GGT linker (amino acids 114-116), LSSmOrange (amino acids 117-352), GGS linker (amino acids 353-355), cpEGFP (amino acids 356-500) and LP linker (amino acids 501-502).

FIGS. 31A and 31B. sfMatryoshCaMP amino acid and DNA sequences (SEQ ID NOs:196 and 197, respectively). M13 peptide (amino acids 1-21), LE linker (amino acids 22-23), cpsfGFP (amino acids 24-112), GGT linker (amino acids 113-115), LSSmOrange (amino acids 116-351), GGS linker (amino acids 352-354), cpEGFP (amino acids 355-498) and LP linker (amino acids 499-500).

FIGS. 32A and 32B. sfMatryoshCaMP-T78H amino acid and DNA sequences (SEQ ID NOs:198 and 199, respectively). M13 peptide (amino acids 1-21), LE linker (amino acids 22-23), cpsfGFP (amino acids 24-112), GGT linker (amino acids 113-115), LSSmOrange (amino acids 116-351), GGS linker (amino acids 352-354), cpEGFP (amino acids 355-498) and LP linker (amino acids 499-500).

FIG. 33. Alignment of fluorescent proteins (GFP (SEQ ID NO:3), EGFP (SEQ ID NO:4), mCerulean (SEQ ID NO:2), mVenus (SEQ ID NO:12), mCherry (SEQ ID NO:14), mApple (SEQ ID NO:18), mRuby2 (SEQ ID NO:94), mKate (SEQ ID NO:16), mKate2 (SEQ ID NO:96) and mRuby (SEQ ID NO:92)), with a bottom row for each alignment of Consistency assigning a number from 0-10 for any position, and indication of potential circular permutation positions. Positions are identified on a scale of from zero (0) to ten (10) from unconserved to conserved, respectively. Boxed regions (spanning aligned amino acid positions 128-148, 155-160, 1168-176 and/or 227-229) are positions of potential insertion of a second protein of the present technology in the circularized first protein according to the GFP numbering.

FIG. 34. Cartoon illustration of position of 138 and 255 in side view (left) and top view (right) of AMT monomer (PDB: 2B2F).

FIG. 35. Alignment of fluorescent proteins (GFP (SEQ ID NO:3), EGFP (SEQ ID NO:4), mCerulean (SEQ ID NO:2), mVenus (SEQ ID NO:12), mCherry (SEQ ID NO:14), mApple (SEQ ID NO:18), mRuby2 (SEQ ID NO:94), mKate (SEQ ID NO:16), mKate2 (SEQ ID NO:96) and mRuby (SEQ ID NO:92)), with a bottom row for each alignment of Consistency assigning a number from 0-10 for any position, and indication of potential circular permutation positions. Positions are identified on a scale of from zero (0) to ten (10) from unconserved to conserved, respectively. Boxed regions (spanning aligned amino acid positions 128-148, 155-160, 1168-176 and/or 227-229) are positions of potential insertion of a second protein of the present technology in the circularized first protein according to the GFP numbering.

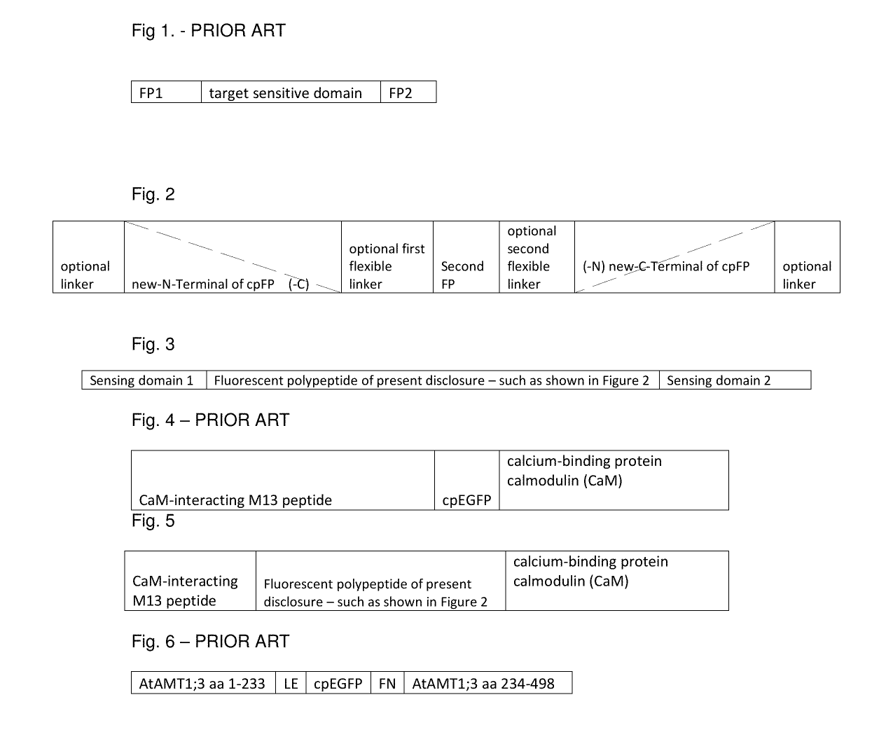

### DESCRIPTION

The present disclosure provides a fluorescent polypeptide containing a fusion of a circularly permuted, first fluorescent protein and a second fluorescent protein, the first fluorescent protein containing a first fluorescent moiety and the second fluorescent protein containing a second fluorescent moiety, wherein the second fluorescent protein is contained in the circularly permuted first fluorescent protein. The fluorescent polypeptide of the presently disclosed technology include a first fluorescent moiety and second fluorescent moiety that may be excited at a single wavelength and that fluoresce at different or distinguishable wavelengths in the fluorescent polypeptide and/or when the fluorescent polypeptide of the present disclosure is included in a sensor or biosensor of the present disclosure.

The present disclosure provides fluorescent polypeptides containing a sensing domain, which may be a circularly permuted single fluorescent protein-based biosensor, and also contains a nested reference domain, wherein the reference domain may be a spectrally distinct unpermuted fluorescent protein. The nested reference domain in embodiments of the presently disclosed technology may be contained within the circularly permuted single fluorescent protein-based biosensor. Fluorescent polypeptides of the present disclosure therefore may include a circularly permuted single fluorescent protein-based biosensor as a first fluorescent protein and a nested reference domain as a second fluorescent protein.

The fluorescent polypeptide of the presently disclosed technology includes a first fluorescent moiety and second fluorescent moiety that may act as partners for Förster Resonance Energy Transfer (FRET). Excitation of the first fluorescent moiety may lead to excitation of the second fluorescent moiety via resonance energy transfer from the first to the second fluorescent moiety or excitation of the second fluorescent moiety may lead to excitation of the first fluorescent moiety via energy transfer from the second to the first fluorescent moiety and fluorescence at different or distinguishable wavelengths in the fluorescent polypeptide and/or when the fluorescent polypeptide of the present disclosure is included in a sensor or biosensor of the present disclosure can be detected.

A fluorescent polypeptide of the presently disclosed technology includes a first optionally circularly permuted fluorescent protein. Specifically, circular permutation entails the interruption of the protein at a new site to form a free amino-(N-) terminus and a free carboxy-(C-) terminus while the original N- and C-termini are linked, such as by a short peptide sequence (such as SEQ ID NO:112). A second fluorescent protein of an embodiment of the presently disclosed technology is joined to the first fluorescent protein by insertion into a loop of the first fluorescent protein. This loop may be the sequence spanning the original N- and C-termini of a circularly permuted first fluorescent protein as in the exemplified sensors presented herein.

A fluorescent polypeptide of the presently disclosed technology may include, as a first fluorescent protein, mCerulean (SEQ ID NO:2), GFP (SEQ ID NO:5), EGFP (SEQ ID NO:4), mVenus (SEQ ID NO:12), T-Sapphire (SEQ ID NO:206), mCherry (SEQ ID NO:14), mKate (SEQ ID NO:16), or mApple (SEQ ID NO:18), which may be circularly permuted as a part of a fluorescent polypeptide of the presently disclosed technology.

A fluorescent polypeptide of the presently disclosed technology may include a native amino-terminus of the first fluorescent protein which is joined to the second fluorescent protein by a second linker and a native carboxy-terminus of the first fluorescent protein is joined to the second fluorescent protein by a first linker, wherein the first linker and the second linker may be the same or different, the first linker and/or said second linker optionally containing a sequence of amino acids. A fluorescent polypeptide of the presently disclosed technology may include an amino-terminus of the first fluorescent protein joined to the carboxy-terminus of the second fluorescent protein and the carboxy-terminus of the first fluorescent protein joined to the amino-terminus of the second fluorescent protein. A fluorescent polypeptide of the presently disclosed technology may include a sequence of amino acids as linker(s) between the first fluorescent protein and the second fluorescent protein, which may be the same or different, and wherein the amino acid sequence of the linker(s) may contain 1, 2, 3, 4, 5, 6, 7, 8, 9, 10, 11, 12, 13, 14, 15, 16, 17, 18, 19 or 20 amino acids. A first linker of a fluorescent polypeptide of the presently disclosed technology and/or a second linker of a fluorescent polypeptide of the presently disclosed technology may be flexible and/or contain an amino acid sequence -Gly-Gly-.

A first linker of a fluorescent polypeptide of the presently disclosed technology and a second linker of a fluorescent polypeptide of the presently disclosed technology, if joined in the absence of the second fluorescent protein, may form an amino acid sequence containing at least one of the following amino acid sequences: GGTGEL (SEQ ID NO:111), GGTGGS (SEQ ID NO:112), FKTRHN (SEQ ID NO:113), GGGGSGGGGS (SEQ ID NO:114), GKSSGSGSESKS (SEQ ID NO:115), GSTSGSGKSSEGKG (SEQ ID NO:116), GSTSGSGKSSEGSGSTKG (SEQ ID NO:117), GSTSGSGKPGSGEGSTKG (SEQ ID NO:118), or EGKSSGSGSESKEF (SEQ ID NO:119). A first linker of a fluorescent polypeptide of the presently disclosed technology and/or a second linker of a fluorescent polypeptide of the presently disclosed technology may contain an amino acid sequence containing at least one of the following amino acid sequences GGT, GEL, GGS, FKT, RHN, GGGGS (SEQ ID NO:120), GKSSGS (SEQ ID NO:121), GSESKS (SEQ ID NO:122), GSTSGSG (SEQ ID NO:123), KSSEGKG (SEQ ID NO:124), GSTSGSGKS (SEQ ID NO:125), SEGSGSTKG (SEQ ID NO:126), GSTSGSGKP (SEQ ID NO:127), GSGEGSTKG (SEQ ID NO:128), EGKSSGS (SEQ ID NO:129), or GSESKEF (SEQ ID NO: 130).

A fluorescent polypeptide of the presently disclosed technology additionally and/or optionally includes a linker(s), such as amino acid sequence linker(s), joined to the free amino-terminus and/or the free carboxy-terminus of the fluorescent polypeptide that are different from the native amino-terminus of the first fluorescent protein and/or the native carboxy-terminus of the first fluorescent protein. The additionally and/or optionally included linker(s) may include an amino acid sequence of 1, 2, 3, 4, 5, 6, 7, 8, 9, 10, 11, 12, 13, 14, 15, 16, 17, 18, 19 or 20 amino acids and may include or contain a combination of naturally-occurring and/or synthetic amino acids or a single naturally-occurring or synthetic amino acid. Linkers of the present disclosed exemplified embodiments include LS, LE, GS, joined to the free amino-terminus or FN, LP joined to the free carboxy-terminus of the fluorescent polypeptide that are different from the native amino-terminus of the first fluorescent protein and/or the native carboxy-terminus of the first fluorescent protein.

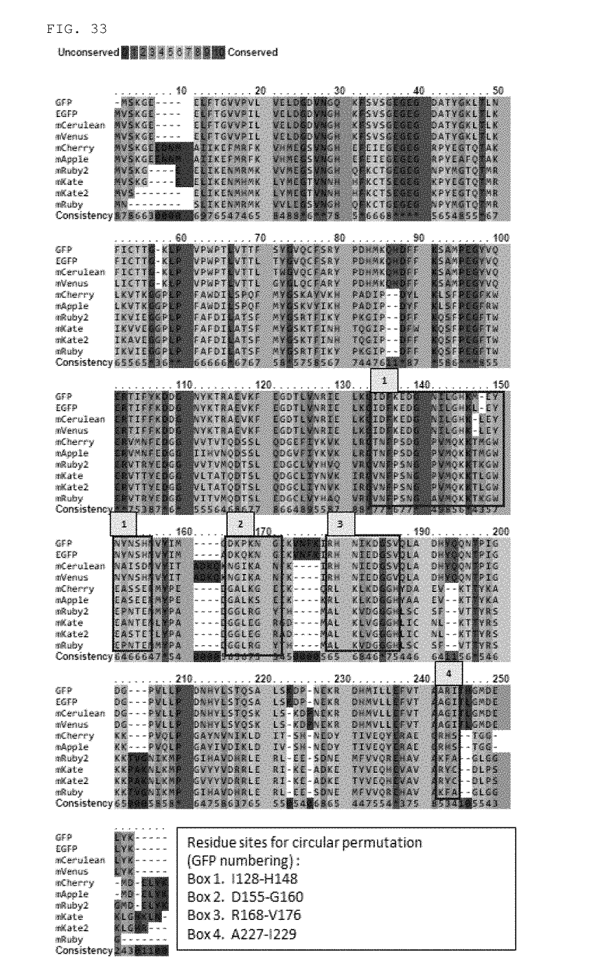

A fluorescent polypeptide of the presently disclosed technology includes circularly permuted, first fluorescent protein wherein the native sequence of the first fluorescent protein may be mCerulean (SEQ ID NO:2), GFP (SEQ ID NO:5), EGFP (SEQ ID NO:4), mVenus (SEQ ID NO:12), T-Sapphire (SEQ ID NO:206), mCherry (SEQ ID NO:14), mKate (SEQ ID NO:16), or mApple (SEQ ID NO:18). The second fluorescent protein of the presently disclosed technology may be nested in the first fluorescent protein at an amino acid position which maintains the fluorescence properties of the first fluorescent protein and the second fluorescent protein. FIG. 33 provides an alignment of the exemplified first fluorescent proteins of the present disclosure and amino acid positions that may be potential insertion sites for the nested second fluorescent second protein.

A fluorescent polypeptide of the presently disclosed technology includes a circularly permuted, first fluorescent protein selected from cpsfGFP (SEQ ID NO:7) or cpEGFP (SEQ ID NO:26), or an alternate form of cpsfGFP (SEQ ID NO:7) or cpEGFP (SEQ ID NO:26) wherein the motif GGTGGS formed from a joining of the second linker and the first linker is GGTGEL (SEQ ID NO:111), GGTGGS (SEQ ID NO:112), FKTRHN (SEQ ID NO:113), GGGGSGGGGS (SEQ ID NO:114), GKSSGSGSESKS (SEQ ID NO:115), GSTSGSGKSSEGKG (SEQ ID NO:116), GSTSGSGKSSEGSGSTKG (SEQ ID NO:117), GSTSGSGKPGSGEGSTKG (SEQ ID NO:118), or EGKSSGSGSESKEF (SEQ ID NO:119), or one of GGT or GGS of the GGTGGS motif is GGT, GEL, GGS, FKT, RHN, GGGGS (SEQ ID NO:120), GKSSGS (SEQ ID NO:121), GSESKS (SEQ ID NO:122), GSTSGSG (SEQ ID NO:123), KSSEGKG (SEQ ID NO:124), GSTSGSGKS (SEQ ID NO:125), SEGSGSTKG (SEQ ID NO:126), GSTSGSGKP (SEQ ID NO:127), GSGEGSTKG (SEQ ID NO:128), EGKSSGS (SEQ ID NO:129), or GSESKEF (SEQ ID NO:130).

A fluorescent polypeptide of the presently disclosed technology may include as a second fluorescent protein a fluorescent protein selected from mVenus (SEQ ID NO:12), LSSmOrange (SEQ ID NO:20), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG.

A fluorescent polypeptide of the presently disclosed technology may contain GO-Matryoshka-LS-FN (SEQ ID NO:30) or a sequence of GO-Matroshka-LS-FN wherein the LSSmOrange (SEQ ID NO:20) motif of SEQ ID NO:30 is mVenus (SEQ ID NO:12), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG.

Examples of combinations of the first and second fluorescent proteins of the presently disclosed fluorescent polypeptides are provided in the following Table 1:

| Circularly permuted | | |
|---|---|---|
| first fluorescent | Second fluorescent | Exemplary literature |
| protein - | protein - | description of individual |
| Fluorophore 1 | Fluorophore 2 | fluorescent proteins |
| cpCerulean | Venus/cpVenus | Meng and Sachs (2012) |
| | LSSmOrange | Shcherbakova et al (2012) |
| | mHoneydew, | Shaner et al (2004) |
| | mBanana, | Shaner et al (2008) |
| | mOrange, | Kredel et al (2009) |
| | dTomato, tdTomato, | Lam et al (2012) |
| | mTangerine, | Eisenstein (2010) |
| | mStrawberry, | Shui et al (2011) |
| | mCherry/cp-mCherry | Yang et al (2013) |
| | mApple | |
| | mRuby/mRuby2 | |
| | mKate2/cp-mKate | |
| | Neptune | |
| | TagRFP-T | |
| | mBeRFP | |
| cpEGFP/cpsfGFP | mRuby2 | Lam et al (2012) |
| | LSS-mKate2 | Piatkevich et al (2010) |
| | mKeima | Kawano et al (2008) |
| | mApple | Eisenstein (2010) |
| | mStrawberry | Tsutsui et al (2008) |
| | Neptune | Shaner et al (2008) |
| | mKOκ | Shui et al (2011) |
| | TagRFP-T | Yang et al (2013) |
| | mCherry/cp-mCherry | |
| | mKate2/cp-mKate | |
| | mBeRFP | |
| cpVenus and | mCherry | Meng and Sachs (2012) |
| cpT-Sapphire | mApple | Hung et al (2011) |
| | mRuby/mRuby2 | Lam et al (2012) |
| | mKate2 | Shaner et al (2004) |
| | mKOκ | Shaner et al (2008) |
| | mOrange | Eisenstein (2010) |
| | Neptune | |
| | TagRFP-T | |
| cp-mCherry/ | cpEGFP/cpsfGFP | Gautam et al (2009) |
| cp-mKate | cpCerulean | Shui et al (2011) |
| | mTurquoise 2 | Goedhart et al (2012) |
| | Clover | Lam et al (2012) |
| | mNeon-Green | Shaner et al (2013) |
| | mUKG | Tsutsui et al (2008) |
| cp-mApple and | cpEGFP/cpsfGFP | Zhao et al (2011) |
| cp-mRuby | cpCerulean | Akerboom et al (2013) |
| | Clover | Shui et al (2011) |
| | mNeon-Green | Goedhart et al (2012) |
| | mUKG | Lam et al (2012) |
| | | Shaner et al (2013) |
| | | Tsutsui et al (2008) |

A fluorescent polypeptide of the presently disclosed technology includes a circularly permuted, first fluorescent protein that is optionally interrupted to form a free amino-terminus and a free carboxy-terminus of the fluorescent polypeptide that are different from the native amino-terminus of the first fluorescent protein and the native carboxy-terminus of the first fluorescent protein, respectively, the circularly permuted, first fluorescent protein being cpsfGFP (SEQ ID NO:7) or cpEGFP (SEQ ID NO:26) and the optional interruption being an amino acid position which maintains the fluorescence properties of the first fluorescent protein and the second fluorescent protein, such as are exemplified in FIG. 33. A fluorescent polypeptide of the presently disclosed technology may have a circularly permuted, first fluorescent protein of cpsfGFP (SEQ ID NO:7) or cpEGFP (SEQ ID NO:26) and the optional interruption to form an amino-terminal end at E142, Y143, Y145, H148, D155, H169, E172, D173, A227 or I229 of the first fluorescent protein, and/or a carboxy-terminal end at N144, N146, N149, K162, K156, N170, I171, D173, E172, A227, or I229, of the first fluorescent protein.

A fluorescent polypeptide of the presently disclosed technology includes a circularly permuted, first fluorescent protein that is optionally interrupted to form a free amino-terminus and a free carboxy-terminus of the fluorescent polypeptide that are different from the native amino-terminus of the first fluorescent protein and the native carboxy-terminus of the first fluorescent protein, respectively, the circularly permuted, first fluorescent protein may be a circularly permuted form of mCerulean (SEQ ID NO:2) and the optional interruption being an amino acid position which maintains the fluorescence properties of the first fluorescent protein and the second fluorescent protein, such as are exemplified in FIG. 33 The optional interruption to form an amino-terminal end may be at G175 of the first fluorescent protein and/or a carboxy-terminal end may be at D174 of the first fluorescent protein (Meng and Sachs, 2012) wherein the native amino-terminus and native carboxy-terminus may be also optionally joined by a second linker and a first linker as described herein.

A fluorescent polypeptide of the presently disclosed technology includes a circularly permuted, first fluorescent protein that is optionally interrupted to form a free amino-terminus and a free carboxy-terminus of the fluorescent polypeptide that are different from the native amino-terminus of the first fluorescent protein and the native carboxy-terminus of the first fluorescent protein, respectively, the circularly permuted, first fluorescent protein being a circularly permuted form of mCherry (SEQ ID NO:14), and the optional interruption being an amino acid position which maintains the fluorescence properties of the first fluorescent protein and the second fluorescent protein, such as are exemplified in FIG. 33, wherein the native amino-terminus and native carboxy-terminus may be also optionally joined by a second linker and a first linker as described herein.

A fluorescent polypeptide of the presently disclosed technology includes a circularly permuted, first fluorescent protein that is optionally interrupted to form a free amino-terminus and a free carboxy-terminus of the fluorescent polypeptide that are different from the native amino-terminus of the first fluorescent protein and the native carboxy-terminus of the first fluorescent protein, respectively, the circularly permuted, first fluorescent protein being a circularly permuted form of mKate (SEQ ID NO:16), and the optional interruption being an amino acid position which maintains the fluorescence properties of the first fluorescent protein and the second fluorescent protein, such as are exemplified in FIG. 33, wherein the native amino-terminus and native carboxy-terminus may be also optionally joined by a second linker and a first linker as described herein.

A fluorescent polypeptide of the presently disclosed technology includes a circularly permuted, first fluorescent protein that is optionally interrupted to form a free amino-terminus and a free carboxy-terminus of the fluorescent polypeptide that are different from the native amino-terminus of the first fluorescent protein and the native carboxy-terminus of the first fluorescent protein, respectively, the circularly permuted, first fluorescent protein being a circularly permuted form of mApple (SEQ ID NO:18), and the optional interruption being an amino acid position which maintains the fluorescence properties of the first fluorescent protein and the second fluorescent protein, such as are exemplified in FIG. 33, wherein the native amino-terminus and native carboxy-terminus may be also optionally joined by a second linker and a first linker as described herein.

A number of methods for identifying insertion sites in fluorescent proteins and/or sensor polypeptides are known in the art, including, for example, site directed mutagenesis, insertional mutagenesis, and deletional mutagenesis. Sites in a sensor polypeptide which can tolerate insertion of a fluorescent polypeptide of the present disclosure can be identified by generating mutant proteins by manipulating the DNA sequence such that a variety of different insertions are produced and screening the mutants by fluorimetric analysis and/or flow cytometry for mutants which retain sensor and fluorescence activity. Such insertions may include replacement of certain amino acids, as well as the addition of a new sequence without a corresponding deletion or replacement in the sequence of the sensor and/or fluorescent protein. Variants identified in this fashion reveal sites which can tolerate insertions while retaining sensor and fluorescence activities.

Additionally, circularly permutation techniques are also useful in identifying sites in fluorescent proteins which are capable of tolerating insertions while retain the ability to fluoresce. Such techniques include are exemplified herein as well as known to those of skill in the art (see, for example, Graf et al., Proc. Natl. Acad. Sci USA, 93:11591-11596 (1996), which is incorporated herein by reference).

In circular permutations, the original N-terminal and C-terminal amino acids of a fluorescent protein are engineered to be linked by a linker moiety. Such linker moieties include those described herein, as well as other easily ascertain by one skilled in the art. This is typically performed at the nucleic acid level resulting in a polynucleotide sequence wherein the 5′ codon encoding the N-terminal amino acid is linked to the 3′ codon encoding the C-terminal amino acid, resulting in a circularized fluorescent protein nucleic acid sequence. The circularized sequence is then cleaved with a nuclease to create a linear polynucleotide sequence, the cleavage site corresponding to an amino acid in of the fluorescent protein. The cleavage of the circularized sequence is either random or specific depending on the desired product, nuclease, and desired sequence. The linearized polynucleotide, which contains sequence homologous to the starting fluorescent protein sequence, is cloned into an expression vector and expressed. The expressed protein sequence is then screened, for example by flow cytometry, for proteins retaining the ability to fluoresce. Accordingly, proteins which retain the ability to fluorescence correspondingly, via identification of the cleavage site, identify amino acids which can tolerate insertions without destroying the ability of the fluorescent protein to fluoresce.

Further provided herein is a fluorescent sensor containing a fluorescent polypeptide of the present disclosure and a sensor, such as a sensor polypeptide.

A fluorescent sensor of the presently disclosed technology may be a ratiometric fluorescent sensor wherein measurement of the fluorescence of the first moiety and the fluorescence of the second moiety upon excitation with said single wavelength and/or due to FRET provides a ratiometric measurement. The present disclosure provides a fluorescent sensor, wherein the sensor polypeptide may be calmodulin or binding fragment thereof, a calmodulin-related protein, recoverin, a nucleoside diphosphate or triphosphate binding protein, an inositol-1,4,5-triphosphate receptor, a cyclic nucleotide receptor, a nitric oxide receptor, a growth factor receptor, a hormone receptor, a ligand-binding domain of a hormone receptor, a steroid hormone receptor, a ligand binding domain of a steroid hormone receptor, a cytokine receptor, a growth factor receptor, a neurotransmitter receptor, a ligand-gated channel, mechanosensitive ion channel, a voltage-gated channel, a protein kinase C, a domain of protein kinase C, a cGMP-dependent protein kinase, an inositol polyphosphate receptor, a phosphate receptor, a carbohydrate receptor, an SH2 domain, an SH3 domain, a PTB domain, an antibody, an antigen-binding site from an antibody, a single-chain antibody, a zinc-finger domain, a protein kinase substrate, a protease substrate, a phosphorylation domain, a redox sensitive loop, Perceval, CH-GECO 2.1, RCaMP, RGECO1, REX-GECO1, Flamindo2, FlincGs, DAG sensor iGluSnFR, HyPer, Ins(1,3,4,5)P4, a maltose sensor, a membrane voltage sensor, peredox, sonar, protein phosphorylation, tandem fluorescent protein timers, rxRFP, a superoxide indicator, ASAP 1, a VSFP, or LOOn-GFP. A fluorescent sensor of the present disclosure includes calmodulin or a calmodulin-related protein moiety as a sensor polypeptide.

A fluorescent sensor of the present disclosure includes, as a sensor polypeptide, a calmodulin-binding domain of skMLCKp, smMLCK, CaMKII, Caldesmon, Calspermin, phosphofructokinase calcineurin, phosphorylase kinase, Ca2+-ATPase 59 kDa PDE, 60 kDa PDE, nitric oxide synthase, type I adenylyl cyclase, Bordetella pertussis adenylyl cyclase, Neuromodulin, Spectrin, MARCKS, F52, .beta.-Adducin, HSP90a, HIV-1 gp160, BBMHBI, Dilute MHC, Mastoparan, Melittin, Glucagon, Secretin, VIP, GIP, or Model Peptide CBP2.

A fluorescent polypeptide or a fluorescent sensor of the presently disclosed technology may include a circularly permuted, first fluorescent protein that further contains a localization sequence.

The presently disclosed technology provides a fluorescent polypeptide wherein the circularly permuted, first fluorescent protein is capable of being made by a method of producing a circularly permuted fluorescent nucleic acid sequence, that includes: linking a nucleic acid sequence encoding a linker moiety to the 5′ nucleotide of a polynucleotide encoding the first fluorescent protein; circularizing the polynucleotide with the nucleic acid sequence encoding the linker sequence; and cleaving the circularized polynucleotide with a nuclease, wherein cleavage linearizes the circularized polynucleotide, and expressing the polynucleotide sequence. The presently disclosed technology provides a fluorescent polypeptide wherein the circularly permuted, first fluorescent protein is capable of being made by a method of producing a circularly permuted fluorescent nucleic acid sequence, involving: linking a nucleic acid sequence encoding a linker moiety to the 5′ nucleotide of a polynucleotide encoding the first fluorescent protein; circularizing the polynucleotide with the nucleic acid sequence encoding the linker sequence; and cleaving the circularized polynucleotide with a nuclease, wherein cleavage linearizes the circularized polynucleotide, and expressing the polynucleotide sequence.

The presently disclosed technology provides a nucleic acid sequence encoding a fluorescent polypeptide of the presently disclosed technology. The presently disclosed technology provides a nucleic acid sequence encoding a fluorescent sensor of the presently disclosed technology.

A nucleic acid sequence encoding a fluorescent polypeptide of the presently disclosed technology may include as a nucleic acid sequence encoding a second fluorescent protein a nucleic acid sequence encoding a second fluorescent protein selected from mVenus (SEQ ID NO:12), LSSmOrange (SEQ ID NO:20), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG. A nucleic acid sequence encoding a fluorescent polypeptide of the presently disclosed technology may include as a nucleic acid sequence encoding a second fluorescent protein a nucleic acid sequence selected from mVenus (SEQ ID NO:11), LSSmOrange (SEQ ID NO:19), mHoneydew (SEQ ID No:21), mBanana (SEQ ID NO:23), mOrange, dTomato (SEQ ID NO:83), tdTomato (SEQ ID NO:85), mTangerine (SEQ ID NO:87), mStrawberry (SEQ ID NO:89), mCherry (SEQ ID NO:13), mApple (SEQ ID NO:17), mRuby (SEQ ID NO:91), mRuby2 (SEQ ID NO:93), mKate2 (SEQ ID NO:95), mNeptune (SEQ ID NO:97), TagRFP-T (SEQ ID NO:99), mBeRFP, LSS-mKate2 (SEQ ID NO:101), mKeima (SEQ ID NO:103), mKOκ (SEQ ID NO:131), mOrange, mTurquoise 2 (SEQ ID NO:105), Clover (SEQ ID NO:107), mNeon-Green (SEQ ID NO:109), or mUKG.

A fluorescent polypeptide of the presently disclosed technology may contain GO-Matroshka-LS-FN (SEQ ID NO:30) or a sequence of GO-Matroshka-LS-FN wherein the LSSmOrange (SEQ ID NO:20) motif of SEQ ID NO:30 is mVenus (SEQ ID NO:12), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG.

A nucleic acid sequence of the present disclosure may encode a fluorescent polypeptide of the presently disclosed technology that contains GO-Matroshka-LS-FN (SEQ ID NO:30) or a polypeptide sequence of GO-Matroshka-LS-FN wherein the LSSmOrange (SEQ ID NO:20) motif of SEQ ID NO:30 is mVenus (SEQ ID NO:12), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG. A nucleic acid sequence of the present disclosure may include a nucleic acid sequence GO-Matroshka-LS-FN (SEQ ID NO:29) or a nucleic acid sequence of GO-Matroshka-LS-FN wherein the LSSmOrange (SEQ ID NO:19) encoding motif of SEQ ID NO:29 is mVenus (SEQ ID NO:11), mHoneydew (SEQ ID No:21), mBanana (SEQ ID NO:23), mOrange, dTomato (SEQ ID NO:83), tdTomato (SEQ ID NO:85), mTangerine (SEQ ID NO:87), mStrawberry (SEQ ID NO:89), mCherry (SEQ ID NO:13), mApple (SEQ ID NO:17), mRuby (SEQ ID NO:91), mRuby2 (SEQ ID NO:93), mKate2 (SEQ ID NO:95), mNeptune (SEQ ID NO:97), TagRFP-T (SEQ ID NO:99), mBeRFP, LSS-mKate2 (SEQ ID NO:101), mKeima (SEQ ID NO:103), mKOκ (SEQ ID NO:131), mOrange, mTurquoise 2 (SEQ ID NO:105), Clover (SEQ ID NO:107), mNeon-Green (SEQ ID NO:109), TSapphire (SEQ ID NO:205) or mUKG.

The presently disclosed technology provides a vector containing a nucleic acid sequence encoding a fluorescent polypeptide of the presently disclosed technology. The presently disclosed technology provides a vector containing a nucleic acid sequence encoding a fluorescent sensor of the presently disclosed technology.

The presently disclosed technology provides a vector containing a nucleic acid sequence encoding a fluorescent polypeptide of the presently disclosed technology and expression control sequences operatively linked to the nucleic acid sequence. The presently disclosed technology provides a vector containing a nucleic acid sequence encoding a fluorescent sensor of the presently disclosed technology and expression control sequences operatively linked to the nucleic acid sequence.

The presently disclosed technology provides for a transgenic non-human animal, plant, bacteria or fungi, isolated animal cell, or plant cell containing a nucleic acid sequence encoding a fluorescent polypeptide of the presently disclosed technology or a nucleic acid sequence encoding a fluorescent sensor of the presently disclosed technology or a vector containing a nucleic acid sequence encoding a fluorescent polypeptide of the presently disclosed technology or a vector containing a nucleic acid sequence encoding a fluorescent sensor of the presently disclosed technology or a vector containing a nucleic acid sequence encoding a fluorescent polypeptide of the presently disclosed technology and expression control sequences operatively linked to the nucleic acid sequence or a vector containing a nucleic acid sequence encoding a fluorescent sensor of the presently disclosed technology and expression control sequences operatively linked to the nucleic acid sequence.

The presently disclosed technology provides for a host cell, such as a prokaryote cell, such as an E. coli., or a eukaryotic cell, such as a yeast cell or a mammalian cell, transfected with an expression vector of the presently disclosed technology.

Biosensors according to the presently disclosed technology may include a sensor polypeptide that is responsive to a chemical, biological, electrical or physiological parameter, and a fluorescent polypeptide wherein the fluorescence of the fluorescent polypeptide is affected by the responsiveness of the sensor polypeptide the responsiveness resulting in protonation or deprotonation of the chromophore of the first fluorescent protein of the fluorescent polypeptide.

The presently disclosed technology provides a method for detecting the presence of an environmental parameter in a sample, by contacting the sample with a fluorescent sensor or biosensor of the present disclosure containing a sensor polypeptide that is responsive to a chemical, biological, electrical, or physiological parameter, and a fluorescent polyprotein as described herein wherein the fluorescence polypeptide is affected by the responsiveness of the sensor polypeptide, and detecting a change in fluorescence wherein a change is indicative of the presence of a parameter which affects the sensor polypeptide. Utilization of FRET based techniques to analyze or detect changes in chemical, biological or electrical parameters may be performed. For example, binding of an analyte such as calcium to a sensor polypeptide such as calmodulin would change the distance or angular orientation of the two fluorescent protein moieties relative to each other and thereby modlulate FRET.

Classes of sensor polypeptides that can be included in sensors or biosensors and/or methods of the presently disclosed technology include, but are not limited to, channel proteins, receptors, enzymes, and G-proteins. Example of sensor polypeptides include calmodulin, a calmodulin-related protein moiety, recoverin, a nucleoside diphosphate or triphosphate binding protein, an inositol-1,4,5-triphosphate receptor, a cyclic nucleotide receptor, a nitric oxide receptor, a growth factor receptor, a hormone receptor, a ligand-binding domain of a hormone receptor, a steroid hormone receptor, a ligand binding domain of a steroid hormone receptor, a cytokine receptor, a growth factor receptor, a neurotransmitter receptor, a ligand-gated channel, a voltage-gated channel, a protein kinase C, a domain of protein kinase C, a cGMP-dependent protein kinase, an inositol polyphosphate receptor, a phosphate receptor, a carbohydrate receptor, an SH2 domain, an SH3 domain, a PTB domain, an antibody, an antigen-binding site from an antibody, a single-chain antibody, a zinc-finger domain, a protein kinase substrate, a protease substrate, a phosphorylation domain, a redox sensitive loop, a loop containing at least two cysteines that can form a cyclic disulfide, and a fluorescent protein moiety.

Channel polypeptides of the presently disclosed technology include, but are not limited to, voltage-gated ion channels including the potassium, sodium, chloride, G-protein-responsive, and calcium channels. A “channel polypeptide” is typically a polypeptide embedded in a cell membrane, and is or is part of a structure that determines what particle sizes and/or charges can traverse the cell membrane. Channel polypeptides include the “voltage-gated ion channels”, which are proteins imbedded in a cell membrane that serve as a crossing point for the regulated transfer of a specific ion or group of ions across the membrane. Specifically, Shaker potassium channels or dihydropuridine receptors from skeletal muscle can be advantageously used in the presently disclosed technology. Several ion channel polypeptides useful in the presently disclosed technology include Human voltage-gated chloride ion channel CLCNS (GenBank accession no X91906), Human delayed rectifier potassium channel (Isk) gene (GenBank accession no L33815), Human potassium channel protein (HPCN3) gene (GenBank accession no M55515), Human potassium channel (HPCN2) (mRNA) (GenBank accession no M55514), Human potassium channel (HPCN1) (mRNA) (GenBank accession no M55513), Human gamma subunit of epithelial amiloride-sensitive sodium channel (mRNA) (GenBank accession no X87160), Human beta subunit of epithelial amiloride-sensitive sodium channel (GenBank accession no X87159).

Channels also include those activated by intracellular signals such as those where the signal is by binding of ligand such as calcium, cyclic nucleotides, G-proteins, phosphoinositols, arachidonic acid, for example, and those where the signal is by a covalent modification such as phosphorylation, enzymatic cleavage, oxidation/reduction, and acetylation, for example. Channel proteins also include those activated by extracellular ligands (e.g., ionotropic receptors). These can be activated by acetylcholine, biogenic amines, amino acids, and ATP, for example.

The sensor or biosensor polypeptide of the presently disclosed technology may include a polypeptide found within or on a cell, often on a membrane, that can combine with a specific type of molecule, e.g., a ligand, and alter a function of the cell. Receptor polypeptides of the presently disclosed technology include, but are not limited to, the growth factor receptors, hormone receptors, cytokine receptors, chemokine receptors, neurotransmitter receptors, ligand-gated channels, and steroid receptors. Sensor polypeptides further include insulin-like growth factor, insulin, somatostatin, glucagon, interleukins, e.g., IL-2, transforming growth factors (TGF-α, TGF-β), platelet-derived growth factor (PDGF), epidermal growth factor (EGF), nerve growth factor (NGF), fibroblast growth factor (FGF), interferon-.gamma. (IFN-.gamma.), and GM-CSF receptors. Receptors such as those where binding of ligand is transmitted to a G-protein (e.g., for 7-transmembrane receptors) or kinase domains (for single transmembrane receptors) can be included as a sensor polypeptide of the presently disclosed technology. These can be activated by acetylcholine, biogenic amines, amino acids, ATP, and many peptides, such as opioids, hypothalamic-releasing hormones, neurohypophyseal hormones, pituitary hormones, tachykinins, secreting, insulins, somatostatins, and gastrointestinal peptides. Exemplary receptor polypeptides that may be sensor polypeptides of the presently disclosed technology include the following: Human insulin receptor gene (Genbank accession No. M29929), Human somatostatin receptor gene (Genbank accession No. L14856), Human IL-2 receptor gene (Genbank accession Nos. X01057, X01058, XD1402), Human TGF receptor (mRNA) (Genbank accession No. M8509), Human PDGF receptor (mRNA) (Genbank accession No. M22734), Human EGF receptor gene (Genbank accession No. X06370), Human NGF receptor (mRNA) (Genbank accession No. M14764), Human FGF receptor (mRNA) (Genbank accession No. M34641), Human GM-CSF receptor (mRNA) (Genbank accession No. M73832), Human IFN-.gamma. receptor (mRNA) (Genbank accession No. X62468).

Examples of uses of the fluorescent sensors of the presently disclosed technology are described in the following Table 2:

Table of cpFP-based sensor as potential examples for improvement with “Matryoshka”

| | | Fluorescent | Modification with the presently | |
|---|---|---|---|---|
| Sensor name | Target | Protein | disclosed technology | Literature |
| Perceval | ATP/ADP | cpmVenus | GO-Matryoshka to replace cpVenus | Berg et al (2009) |
| CH-GECO 2.1 | Ca2+ | cp-mCherry | sfGFP could be inserted into the cp- | Carlson and Campbel |
| | | | mCherry using the same insertion loop | (2013) |
| | | | as presented in GO-Matryoshka | |
| RCaMP | Ca2+ | cp-mRuby | sfGFP could be inserted into the cp- | Akerboom et al (2013) |
| | | | mRuby | |
| RGECO1 | Ca2+ | cp-mApple | sfGFP could be inserted into the cp- | Zhao et al., 2011 |
| | | | mApple | |
| REX-GECO1 | Ca2+ | LSS RFP | sfGFP could be inserted into the | Wu et al (2014) |
| | | | LSSRFP | |
| Flamindo2 | cAMP | Citrine | GO-Matryoshka to replace Citrine | Odaka et al (2014) |
| FlincGs | cGMP | cpEGFP | GO-Matryoshka to replace cpEGFP | Nausch et al (2008) |
| DAG sensor | Diacylglycerol | cpGFP | GO-Matryoshka to replace GFP | Tewson et al (2012) |
| | (DAG) | | | |
| iGluSnFR | Glutamate | cpGFP | GO-Matryoshka to replace cpGFP | Marvin et al (2013) |
| HyPer | Hydrogen | cpYFP | GO-Matryoshka to replace cpYFP | Belousov et al (2006) |
| | Peroxide | | | |
| Ins(1,3,4,5)P4 | inositol-1,3,4,5- | cpGFP | GO-Matryoshka to replace cpGFP | Sakaguchi et al (2009) |
| | tetrakisphosphate | | | |
| Maltose sensors | Maltose | Different | GO-Matryoshka to replace cpGFP | Marvin et al (2011) |
| | | cpFP | | |
| Membrane | Membrane voltage | GFP variant | GO-Matryoshka to replace GFP | Siegel and Isacoff |
| voltage sensor | | | | (1997) |
| Peredox | NAD+/NADH | cpFP T- | mCherry could be inserted into the | Hung et al (2011) |
| | | Sapphire | cpFP T-Sapphire (was tried as tandem | |
| | | | fusion) | |
| Sonar | NAD+/NADH | cpYFP | GO-Matryoshka to replace cpYFP or | Zhao et al (2015) |
| | | | cyan FP insertion into cpYFP | |
| Protein | Protein | Different | GO-Matryoshka to replace cpFP | Kawai et al (2004) |
| Phosphorylation | Phosphorylation | cpFP color | | |
| | | variants | | |
| Tandem | Protein turnover | sfGFP and | GO-Matryoshka could be used as timer | Khmelinskii et al |
| fluorescent | | mCherry | itself due to different maturation times | (2012) |
| protein timers | | fusion | of cpsfGFP (fast) and LSSmOrange | |
| | | | (slow) | |
| rxRFP | Redox | cpRFP | sfGFP could be inserted into the cpRFP | Fan et al (2015) |
| Superoxide | superoxide | cpYFP | GO-Matryoshka to replace cpYFP or | Wang et al (2008) |
| indicators | | | cyan FP insertion into cpYFP | |
| ASAP 1 | Voltage | cpsfGFP | GO-Matryoshka to replace cpsfGFP | St. Pierre et al (2014) |
| VSFPs | Voltage | cpEGFP | GO-Matryoshka to replace cpEGFP or | Gautam et al (2009) |
| | | and mKate | sfGFP could be inserted into the | |
| | | | cpmKate | |
| LOOn-GFP | | cpGFP, | LSSmOrange could be inserted into the | Huang et al (2015) |
| | | truncated | cpGFP version | |

The responsiveness of a sensor polypeptide (e.g. a change in conformation or state) that occurs in response to interaction of the sensor polypeptide with a chemical, biological, electrical or physiological parameter can cause a change in fluorescence of the fluorescence polypeptide of the presently disclosed technology. The change can be the result of an alteration in the environment, structure, protonation or oligomerization status of the fluorescent indicator or chromophore. The optical properties (e.g., fluorescence) of the indicator that can be altered in response to the conformational change in the sensor polypeptide include, but are not limited to, changes in the excitation or emission spectrum, quantum yield, extinction coefficient, excited-state lifetime and degree of self-quenching for example. The cause of the changes in these parameters can include, but are not limited to, changes in the environment, changes in the rotational or vibrational freedom of the fluorescent protein in the sensor, changes in the angle of the fluorescent proteins in the sensor with respect to the exciting light or the optical detector apparatus, changes in the protonation or deprotonation of amino acids or side groups associated with and/or part of a chromophore, changes in the solvent accessibility to the chromophore, changes to the excited-state proton transfer pathway, or changes in distance or dipole orientation between fluorescent proteins in the sensors on associated responsive polypeptides.

In the fluorescent sensor of the presently disclosed technology, a fluorescent polypeptide of the present disclosure is operably inserted in the sensor polypeptide. Detection or measurement of fluorescence or a fluorescent property of the fluorescent sensor of the presently disclosed technology provides a means of detecting the responsiveness of the sensor. Fluorescent properties of the fluorescent sensor that may be detected or measured include molar extinction coefficient at an appropriate excitation wavelength, the fluorescence quantum efficiency, the shape of the excitation spectrum or emission spectrum, the excitation wavelength maximum and emission wavelength maximum, the ratio of excitation amplitudes at two different wavelengths, the ratio of emission amplitudes at two different wavelengths, the excited state lifetime, or the fluorescence anisotropy. A measurable difference in any one of these properties between the active and inactive states of the fluorescent polypeptide of the fluorescent sensor of the presently disclosed technology may be useful in detecting and/or measuring a response of the sensor in assays for activity. A measurable difference can be determined by determining the amount of any quantitative fluorescent property, e.g., the amount of fluorescence at a particular wavelength, or the integral of fluorescence over the emission spectrum.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. For example, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. Other means of measuring fluorescence can also be used with the invention.

Methods of performing assays on fluorescent materials are well known in the art and are described in, e.g., Lakowicz, Principles of Fluorescence Spectroscopy, Plenum Press (1983); Herman, Resonance energy transfer microscopy, in: Fluorescence Microscopy of Living Cells in Culture, Part B. Methods in Cell Biology, vol. 30, ed. Taylor & Wang, San Diego: Academic Press (1989), pp. 219-243; Turro, Modern Molecular Photochemistry, Menlo Park: Benjamin/Cummings Publishing Col, Inc. (1978), pp. 296-361.

As noted above and herein, the fluorescent polypeptide of the presently disclosed technology provides a basis for a ratiometric measurement or detection wherein fluorescence of the fluorescent first protein may be compared to the fluorescence of the fluorescent second protein in real time.

Combinations of fluorescent first and second proteins in fluorescent polypeptides of the presently disclosed technology make it possible to use a single fluorescent excitation wavelength to generate separate and distinguishable fluorescent emission wavelengths which may be reported in a ratiometric manner. Combinations of fluorescent first and second proteins in fluorescent polypeptides of the presently disclosed technology may alternatively be detected and/or measured with separate fluorescent excitation wavelengths for the first fluorescent protein and the second fluorescent protein to generate separate and distinguishable fluorescent emission wavelengths which may be reported in a ratiometric manner.

Fluorescent polypeptides of the presently disclosed technology may be excited with a single fluorescent excitation wavelength(s) in the range of 400-800 nm in a manner known or determinable to produce separate and distinguishable fluorescent emission wavelengths in the range of 400-800 nm that is distinguishable from the excitation wavelength(s).

Alternatively, fluorescent polypeptides of the presently disclosed technology may be excited with separate fluorescent excitation wavelengths in the ranges of 400-800 nm to produce separate and distinguishable fluorescent emission wavelengths in the ranges of 400-800 nm.

The fluorescent polypeptides of the presently disclosed technology may be produced as chimeric proteins by recombinant DNA technology. Recombinant production of fluorescent proteins and polypeptides involves expressing nucleic acids having sequences that encode the proteins and polypeptides. Nucleic acids encoding fluorescent proteins and polypeptides are described herein and may be transcribed and translated by methods known in the art. Mutant versions of fluorescent proteins can be made by site-specific mutagenesis of other nucleic acids encoding fluorescent proteins, such as those described herein, or by random mutagenesis caused by increasing the error rate of PCR of the original polynucleotide with 0.1 mM MnCl₂ and unbalanced nucleotide concentrations, for example.

In the chimeric proteins or the fluorescent sensors of the presently disclosed technology, the fluorescent polypeptide is operably inserted into the sensor polypeptide, which responds (e.g., a conformation change), for example, to a cell signaling event. Cell signaling events that occur in vivo can be of very short duration. The fluorescent sensors of the presently disclosed technology allow measurement and/or detection of the optical parameter, such as fluorescence, which is altered in response to the cell signal, for example, over the same time period that the event actually occurs. Alternatively, the response can be measured after the event occurs (over a longer time period) as the response that occurs in a fluorescent sensor of the disclosure may be of a longer duration than the cell signaling event itself. In either embodiment, the presence of the second fluorescent protein of the fluorescent polypeptide of the present disclosure provides for a ratiometric determination of the response of the fluorescent sensor of the presently disclosed technology.

Polynucleotide and nucleic acid sequences are a polymeric form of nucleotides at least 2 bases in length. An isolated nucleic acid sequence is a polynucleotide that is no longer immediately contiguous with both of the coding sequences with which it was naturally and immediately contiguous (one on the 5′ end and one on the 3′ end) in the naturally occurring genome of the organism from which it is derived or may be found. Isolated nucleic acid sequences includes, for example, a recombinant DNA, which can be incorporated into a vector, including an autonomously replicating plasmid or virus; or into the genomic DNA of a prokaryotic or eukaryotic cell or organism; or that exists as a separate molecule (e.g. a cDNA) independent of other sequences. The nucleotides of the presently disclosed technology can be ribonucleotides, deoxyribonucleotides, or modified forms thereof, and the polynucleotides can be single stranded or double stranded.

A nucleic acid sequence of the presently disclosed technology may be operatively linked to expression control sequences or juxtaposed wherein the components so described are in a relationship permitting them to function in their intended manner. An expression control sequence operatively linked to a coding sequence is ligated such that expression of the coding sequence is achieved under conditions compatible with the expression control sequences.

Expression control sequences are nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding nucleic acid sequence, splicing signals for introns, maintenance of the correct reading frame of that gene to permit proper translation of the mRNA, and stop codons. Nucleic acid sequences of the present disclosure listed as including stop codons, such as in the figures and sequences, may be optionally excluded from the described sequence when used in a construct of the presently disclosed technology in a manner recognized by those of ordinary skill and sequences described in the figures and sequences as including stop codons are similarly described herein as not operatively not containing any included stop codons.

Control sequences include, at a minimum, components whose presence can influence expression, and can also include additional components whose presence is advantageous, for example, leader sequences and chimeric partner sequences. Expression control sequences can include a promoter.

A promoter is a minimal sequence sufficient to direct transcription. Also included in the presently disclosed technology are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the presently disclosed technology (see e.g., Bitter et al., 1987, Methods in Enzymology 153:516-544). For example, when cloning in bacterial systems, inducible promoters such as pL of bacteriophage .gamma., plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter; CMV promoter) may be used. Promoters produced by recombinant DNA or synthetic techniques may also be used to provide for transcription of the nucleic acid sequences of the presently disclosed technology.

Fluorescent proteins of the presently disclosed technology may include proteins capable of emitting light when excited with appropriate electromagnetic radiation, and which has an amino acid sequence that is either natural or engineered and may be derived from the amino acid sequence of an Aequorea-related fluorescent protein. Fluorescent indicators of the presently disclosed technology may include a fluorescent protein having a sensor polypeptide whose emitted light varies with the response state or conformation of the sensor polypeptide upon interaction with a chemical, biological, electrical or physiological parameter. Fluorescent indicators of the present disclosure may also alternatively include a fluorescent protein whose amino acid sequence has been circularly permuted. The fluorescent indicators of the presently disclosed technology may also or alternatively be sensitive to pH in the range of about 5 to about 10.

The presently disclosed technology additionally includes functional fragments of fluorescent polypeptides and fluorescent proteins and sensor polypeptides described herein. Functional fragments are fluorescent polypeptides and fluorescent proteins and sensor polypeptides which possesses biological function or activity which is identified through a defined functional assay.

Minor modifications of the fluorescent polypeptides, fluorescent proteins and/or fluorescent sensors of the presently disclosed technology can result in polypeptides and/or proteins that have substantially equivalent activity as compared to the unmodified counterpart polypeptide and/or protein as described herein. Such modifications may be deliberate, as by site-directed mutagenesis, or may be spontaneous. All of the polypeptides and proteins produced by these modifications are included herein as long as fluorescence of the polypeptide, proteins and/or sensor exists.

Substantially identical or substantially homologous polypeptides, proteins and/or sensors of the presently disclosed polypeptides, proteins and/or sensors are additionally included in the present description, such being a protein or polypeptide that retains the activity of a polypeptides, proteins and/or sensors, or nucleic acid sequence or polynucleotide encoding the same, and which exhibits at least 80%, 85%, 90%, 95%, 97%, 98% or 99% homology or identity to a reference amino acid or nucleic acid sequence. For polypeptides, the length of comparison sequences will generally be along the entire sequence or functional fragment of the protein (such as the first fluorescent proteins or second fluorescent proteins described herein) or polypeptide (such as the fluorescent polypeptides or fluorescent sensors described herein). For nucleic acids, the length of comparison sequences will generally be along the entire sequence encoding the protein, polypeptide or functional fragment of the protein (such as the first fluorescent proteins or second fluorescent proteins described herein) or polypeptide (such as the fluorescent polypeptides or fluorescent sensors described herein).

Substantially identical amino acid sequences additionally or alternatively differ by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein or polypeptide (e.g., assayed as described herein). Homology may be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

Proteins or polypeptides described herein may be purified or substantially purified. Substantially pure proteins or polypeptides include proteins or polypeptides which have been separated from components which naturally accompany it. Typically, the protein or polypeptide is substantially pure when it is at least 60%, 75%, 85%, 95%, 97%, 98% or 99% by weight, free from the proteins and naturally-occurring organic molecules with which it is naturally associated. A substantially pure protein or polypeptide may be obtained, for example, by extraction from a natural source (e.g., a plant cell); by expression of a recombinant nucleic acid encoding a functional engineered fluorescent protein; or by chemically synthesizing the protein. Purity may be measured by any appropriate method, e.g., column chromatography, polyacrylamide gel electrophoresis, or HPLC analysis.

A protein or polypeptide is substantially free of naturally associated components when it is separated from those contaminants which accompany it in its natural state. Thus, a protein or polypeptide which is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

The presently disclosed technology provides polynucleotides encoding the fluorescent proteins, polypeptides and sensors described herein. These polynucleotides include DNA, cDNA, and RNA sequences. Such polynucleotides include naturally occurring, synthetic, and intentionally manipulated polynucleotides. For example, the polynucleotide may be subjected to site-directed mutagenesis. The polynucleotides of the presently disclosed technology include sequences that are degenerate as a result of the genetic code. Therefore, all degenerate nucleotide sequences are included in the presently disclosed technology as long as the amino acid sequence of the proteins, polypeptides and sensors described herein encoded by the nucleic acid sequences are functionally unchanged.

Protein, polypeptide and sensors included herein may also include a targeting sequence to direct the fluorescent proteins, fluorescent polypeptides and/or fluorescent sensors of the presently disclosed technology to particular cellular sites by fusion to appropriate organellar targeting signals or localized host proteins. A polynucleotide encoding a targeting sequence can be ligated to the 5′ terminus of a polynucleotide encoding the fluorescent proteins, fluorescent polypeptides and/or fluorescent sensors such that the targeting peptide is located at the amino terminal end of the resulting fusion polynucleotide/polypeptide. The targeting sequence can be, e.g., a signal peptide. In the case of eukaryotes, the signal peptide is believed to function to transport the fusion polypeptide across the endoplasmic reticulum. The secretory protein is then transported through the Golgi apparatus, into secretory vesicles and into the extracellular space or, preferably, the external environment. Signal peptides which can be utilized according to the invention include pre-pro peptides which contain a proteolytic enzyme recognition site. Other signal peptides with similar properties are known to those skilled in the art, or can be readily ascertained using well known and routine methods.

In the presently disclosed technology, the nucleic acid sequences encoding the fluorescent proteins, fluorescent polypeptides and/or fluorescent sensors may be inserted into a recombinant expression vector. Recombinant expression vectors include plasmids, virus or other vehicle known in the art that has been manipulated by insertion or incorporation of the nucleic acid sequences encoding the chimeric peptides of the presently disclosed technology. The expression vector typically contains an origin of replication, a promoter, as well as specific genes which allow phenotypic selection of the transformed cells. Vectors suitable for use in the presently disclosed technology include, but are not limited to the T7-based expression vector for expression in bacteria (Rosenberg et al., Gene, 56:125, 1987), the pMSXND expression vector, or adeno or vaccinia viral vectors for expression in mammalian cells (Lee and Nathans, J. Biol. Chem., 263:3521, 1988), baculovirus-derived vectors for expression in insect cells, cauliflower mosaic virus, CaMV; tobacco mosaic virus, TMV.

The nucleic acid sequences encoding a fluorescent protein, fluorescent polypeptide and/or fluorescent sensor of the presently disclosed technology may also include a localization sequence to direct the fluorescent protein, fluorescent polypeptide and/or fluorescent sensor to particular cellular sites by fusion to appropriate organellar targeting signals or localized host proteins. A polynucleotide encoding a localization sequence, or signal sequence, can be ligated or fused at the 5′ terminus of a polynucleotide encoding the fluorescent protein, fluorescent polypeptide and/or fluorescent sensor such that the signal peptide is located at the amino terminal end of the resulting chimeric polynucleotide/polypeptide. In the case of eukaryotes, the signal peptide is believed to function to transport the chimeric polypeptide across the endoplasmic reticulum. The secretory protein is then transported through the Golgi apparatus, into secretory vesicles and into the extracellular space or, preferably, the external environment. Signal peptides that can be utilized according to the presently disclosed technology include pre-propeptides which contain a proteolytic enzyme recognition site. Other signal peptides with similar properties to those described herein are known to those skilled in the art, or can be readily ascertained without undue experimentation. The localization sequence can be a nuclear localization sequence, an endoplasmic reticulum localization sequence, a peroxisome localization sequence, a mitochondrial localization sequence, or a localized protein. Localization sequences can be targeting sequences which are described, for example, in “Protein Targeting”, Chapter 35 of Stryer, Biochemistry (4th ed.), W. H. Freeman, 1995. The localization sequence can also be a localized protein. Some important localization sequences include those targeting the nucleus (KKKRK (SEQ ID NO:158)), mitochondrion (amino terminal MLRTSSLFTRRVQPSLFRNILRLQST-; (SEQ ID NO:159)), endoplasmic reticulum (KDEL; (SEQ ID NO:160)) at C-terminus, assuming a signal sequence present at N-terminus), peroxisome (SKF at C-terminus), synapses (S/TDV or fusion to GAP 43, kinesin and tau) prenylation or insertion into plasma membrane (CAAX (SEQ ID NO:161), CC, CXC, or CCXX (SEQ ID NO:162) at C-terminus), cytoplasmic side of plasma membrane (chimeric to SNAP-25), or the Golgi apparatus (chimeric to furin). The construction of expression vectors and the expression of genes in transfected cells involves the use of molecular cloning techniques also well known in the art (see, for example, Sambrook et al., Molecular Cloning: A Laboratory Manual (Cold Spring Harbor Laboratory Press 1989); and Current Protocols in Molecular Biology, Ausubel et al., eds. (Greene Publishing Associates, Inc., and John Wiley & Sons, Inc., 1994, and most recent Supplement). These methods include in vitro recombinant DNA techniques, synthetic techniques and in vivo recombination/genetic recombination. (See, for example, Sambrook et al., supra, 1989).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the chimeric polypeptides of the present disclosure, and a second foreign DNA molecule encoding a selectable phenotype, such as the herpes simplex thymidine kinase gene. Another method is to use a eukaryotic viral vector, such as simian virus 40 (SV40) adenovirus, vaccinia virus, or bovine papilloma virus, to transiently infect or transform eukaryotic cells and express the protein. (Eukaryotic Viral Vectors, Cold Spring Harbor Laboratory, Gluzman ed., 1982). Methods of stable transfer, meaning that the foreign DNA is continuously maintained in the host, are known in the art.

Eukaryotic systems, including mammalian expression systems, allow for proper post-translational modifications of expressed mammalian proteins to occur. Eukaryotic cells which possess the cellular machinery for proper processing of the primary transcript, glycosylation, phosphorylation, and optionally secretion of the gene product may be used as host cells for the expression of the fluorescent protein, polypeptides and/or sensors. Such host cell lines may include but are not limited to CHO, VERO, BHK, HeLa, COS, MDCK, Jurkat, HEK-293, and WI38.

Mammalian cell systems which utilize recombinant viruses or viral elements to direct expression may be engineered. For example, when using adenovirus expression vectors, the nucleic acid sequences encoding a fluorescent protein, polypeptide and/or sensor of the present disclosure may be ligated to an adenovirus transcription/translation control complex, e.g., the late promoter and tripartite leader sequence. This nucleic acid sequence may then be inserted in the adenovirus genome by in vitro or in vivo recombination. Insertion in a non-essential region of the viral genome (e.g., region E1 or E3) will result in a recombinant virus that is viable and capable of expressing the fluorescent protein, polypeptide and/or sensor in infected hosts (see, for example, Logan & Shenk, Proc. Natl. Acad. Sci. USA, 81: 3655-3659, 1984). Alternatively, the vaccinia virus 7.5K promoter may be used (see, for example, Mackett et al., Proc. Natl. Acad. Sci. USA, 79: 7415-7419, 1982; Mackett et al., J. Virol. 49: 857-864, 1984; Panicali et al., Proc. Natl. Acad. Sci. USA 79: 4927-4931, 1982). Vectors based on bovine papilloma virus which have the ability to replicate as extrachromosomal elements (Sarver et al., Mol. Cell. Biol. 1: 486, 1981) may be used.

The presently disclosed technology includes a method for determining the presence of a chemical, biological, electrical or physiological parameter, by contacting the sample with a fluorescent sensor of the present disclosure; exciting the sensor; and measuring the amount of an optical property of the fluorescent polypeptide in the presence and absence of a parameter, such that a change in the optical property is indicative of an effect of the parameter on the fluorescent polypeptide. A series of standards, with known levels of activity, can be used to generate a standard curve or the second fluorescent protein of the fluorescent polypeptide of the fluorescent sensor may be used as an internal and ratiometric control. The optical event, such as change in intensity of fluorescence, that occurs following exposure of a sample to the change in environmental condition that is detected by the sensor of the present disclosure is measured, and the amount of the optical property is then compared to the standard curve or the second fluorescent protein of the fluorescent polypeptide of the fluorescent sensor may be used as an internal and ratiometric control. A standard, with a known level of activity or concentration, may be used to generate a standard curve, or to provide reference standards.

The presently disclosed technology provides methods for determining transient changes in a chemical, biological, electrical or physiological parameter, by contacting the sample with a fluorescent sensor of the present disclosure and measuring or detecting a change in the optical property of the fluorescent sensor over time.

The presently disclosed technology provides screenings assays to determine whether a compound (e.g., a drug, a chemical or a biologic) alters the properties of the fluorescent sensor polypeptide of the present disclosure. The assay may be performed on a sample containing the chimeric protein or fluorescent sensor of the disclosure in vitro or in vivo.

In one embodiment, the assay is performed on a sample containing the fluorescent sensor of the present disclosure in vitro. The fluorescent sensor of the present disclosure is mixed with a known amount of analyte (e.g. calcium) and the optical properties, such as fluorescence properties, are assessed. The difference in fluorescence properties of the fluorescent sensor in absence and presence of analyte (e.g. calcium) is indicative of fluorescent sensor response.

In another embodiment, the ability of a compound to alter the activity of a particular protein (i.e., a sensor polypeptide) in vivo is determined. In an in vivo assay, cells transfected with an expression vector encoding the fluorescent sensor of the present disclosure are exposed to different amounts of the test analyte (e.g. ammonium), and the effect on the optical parameter, such as fluorescence, in each cell or a pool of cells can be determined. Typically, the difference is calibrated against standard measurements to yield an absolute amount of fluorescent sensor activity and analyte concentration. In a given cell type, any measurable change between activity in the presence of the analyte (e.g. ammonium) as compared with the activity in the absence of the analyte (e.g. ammonium), is indicative of fluorescent sensor response.

The disclosed technology additionally provides kits for determining the presence of an activity and/or analyte in a sample. Such a kit may contain a container containing a chimeric protein comprising a fluorescent sensor polypeptide, or fragment thereof, which is affected by a change in a parameter or the environment, wherein optical properties of the sensor are altered in response to the change. In another embodiment, a kit of the invention contains an isolated nucleic acid sequence which encodes a chimeric protein comprising an optically active polypeptide having operatively inserted therein a sensor polypeptide, or fragment thereof, which is affected by a change in a parameter or the environment, wherein optical properties of the sensor are altered in response to the change. The nucleic acid sequence of the later kit may be contained in a host cell, preferably stably transfected. The cell could optionally be transiently transfected. Thus, the cell acts as an indicator kit in itself. Screening of the optical properties, such as fluorescence properties, of the fluorescent sensor alone or expressed by a host cell can determine the presence of sensor activity and/or quantify the analyte in a sample.

The presently disclosed technology provides transgenic, non-human, animals that have cells that express a fluorescent sensor or fluorescent polypeptide as described herein. Such non-human animals include vertebrates such as rodents, non-human primates, sheep, dog, cow, pig, amphibians, reptiles and fish. Such transgenic animals may be produced by introducing transgenes into the germline of the non-human animal. Embryonal target cells at various developmental stages can be used to introduce transgenes. Different methods are used depending on the stage of development of the embryonal target cell. The zygote is the best target for micro-injection. In the mouse, the male pronucleus reaches the size of approximately 20 micrometers in diameter which allows reproducible injection of 1-2 picoliters of DNA solution. The use of zygotes as a target for gene transfer has a major advantage in that in most cases the injected DNA will be incorporated into the host gene before the first cleavage (Brinster et al., Proc. Natl. Acad. Sci. USA 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

Viral infection can also be used to introduce transgene into a non-human animal (e.g., retroviral, adenoviral or any other RNA or DNA viral vectors). The developing non-human embryo can be cultured in vitro to the blastocyst stage. During this time, the blastomeres can be targets for retro viral infection (Jaenich, R., Proc. Natl. Acad. Sci USA 73:1260-1264, 1976). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner, et al., Proc. Natl. Acad. Sci. USA 82:6927-6931, 1985; Van der Putten, et al., Proc. Natl. Acad. Sci USA 82:6148-6152, 1985). Transfection is easily and efficiently obtained by culturing the blastomeres on a monolayer of virus-producing cells (Van der Putten, supra; Stewart, et al., EMBO J. 6:383-388, 1987). Alternatively, infection can be performed at a later stage. Virus or virus-producing cells can be injected into the blastocoele (D. Jahner et al., Nature 298:623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retro viral infection of the midgestation embryo (D. Jahner et al., supra).

The presently disclosed technology is exemplified by the following non-limiting examples.

### Examples

GO-Matryoshka

In order to design a construct suitable for one step generation of ratiometric sensors with large dynamic range, cpsfGFP was fused with LSSmOrange. The sensing domain cpEGFP is commonly used in single-FP biosensors, due to the large dynamic range of the intensiometric response to conformational changes in its environment. While retaining the sensitivity of cpEGFP, cpsfGFP is more stable and tolerant to insertions and has demonstrated improved brightness compared to cpEGFP²². As a nested reference fluorescent protein, LSSmOrange was chosen because of its brightness and pH-stability. Both fluorescent proteins are spectrally distinct with little fluorescence emission spectral overlap.

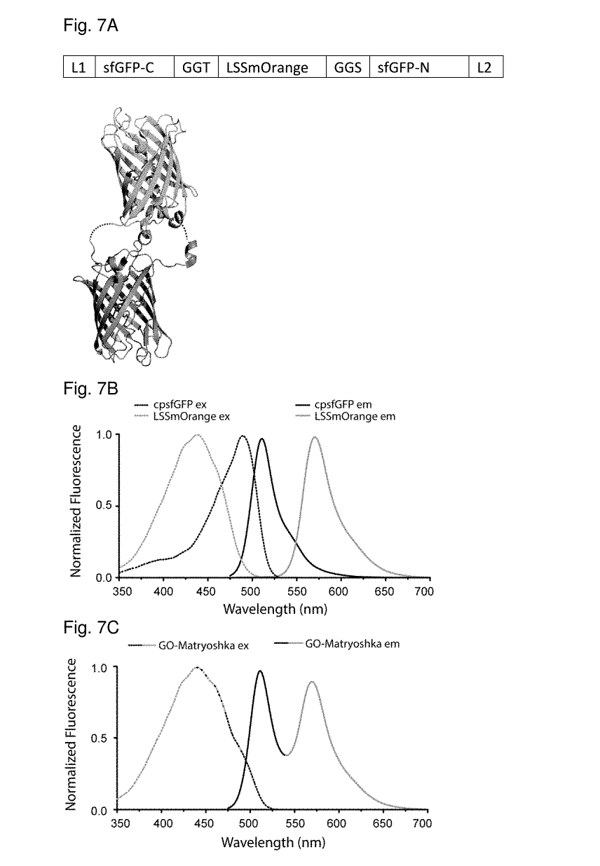

LSSmOrange was inserted into the middle of the GGT-GGS sequence, which connects the original N- and C-terminus of the superfolder GFP^{23,24} (FIG. 7A). The combination of cpsfGFP and LSSmOrange is referred to herein as GO-Matryoshka. The N- and C-terminal residues flanking GO-Matryoshka are known to affect the protonation equilibrium of the chromophore and thus the fluorescence properties of the cpFP. They act as direct connection point when the cpFP is connected with a sensor domain and can impact the dynamic range and fluorescence intensity (FI) of the sensor. Therefore, the flanking residues where maintained when characterizing GO-Matryoshka. The selected residues were leucine and serine (LS) as N-terminal amino acids and phenylalanine and arginine (FN) as C-terminal amino acids, since the LS/FN combination had proved among the best combinations during AmTrac design¹². In vitro characterization of purified GO-Matryoshka revealed dual-emission behavior with two emission maxima at λₑₘ˜510 nm and λₑₘ˜570 nm upon excitation with λ_{exc}˜440 nm and single emission maximum at λₑₘ˜510 nm upon λ_{exc} 488 nm excitation. The intensity of the green emission (λ_{exc}˜485 nm; λₑₘ˜510 nm) of cpsfGFP and GO-Matryoshka showed an increased fluorescence intensity (FI) amplitude (brightness) for GO-Matryoshka of 15% compared to cpsfGFP as shown in the following Table 3.

| | Brightness (%) | pKₐ |
|---|---|---|
| cpsfEGFP | 100 | 6.58 ± 0.04 |
| GO-Matryoshka | 115 ± 8 | 6.61 ± 0.11 |

Table 3 provides the relative brightness of the green emission (λ_{exc} 485 nm; λₑₘ˜510 nm) and pKₐ values of cpsfGFP and GO-Matroshka—brightness as green emission at pH 9, relates to cpsfGFP

Comparative analysis of the steady-state spectral properties of the individual fluorescent proteins (FPs) revealed that GO-Matryoshka exhibited no detectable change in the excitation or emission maxima as compared to cpsfGFP and LSSmOrange (FIGS. 7B and 7C). To test if the LSSmOrange insertion affected the pH sensitivity, pH titration of cpsfGFP and GO-Matryoshka were performed and revealed pKₐ values of ˜6.6 for both cpsfGFP and GO-Matryoshka (Table 3).

### Conversion of the Calcium Sensor GCaMP6s into a Ratiometric Calcium Sensor Employing the “Matryoshka” Technology

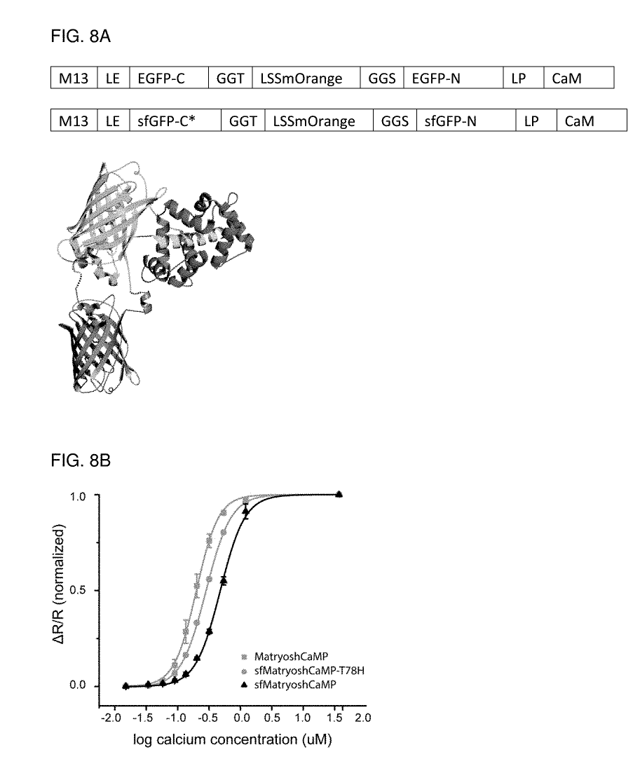

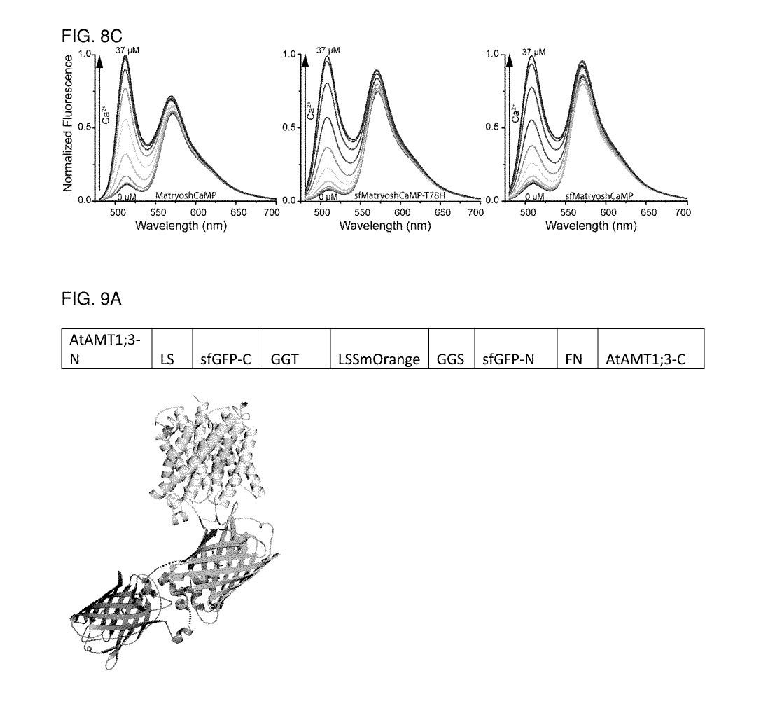

To demonstrate the utility of GO-Matryoshka, GCaMP6s served as template to generate three different MatryoshCaMP variants: (1) MatryoshCaMP contained the LSSmOrange (reference domain) inserted into the GGT-GGS linker of the cpEGFP (sensing domain), (2) sfMatryoshCaMP contained the GO-Matryoshka cassette instead of the cpEGFP, and (3) sfMatryoshCaMP-T78H contained a histidine instead of threonine in amino acid position 78 of cpsfGFP (FIG. 8A). In addition to LSSmOrange, four red fluorescent protein variants were tested. Excitation at λexc480 nm led to green and red emission due to FRET from the cpsfGFP to the red fluorescent proteins. However the ratio of the spectral red maxima over green maxima was the highest for the LSSmOrange-containing construct. Therefore, the study was continued with the LSSmOrange-based construct. In GCaMP6s a histidine at position 78 was identified to be beneficial for the sensitivity of the sensor. As a control, the cpEGFP in GCaMP6s was replaced by cpsfGFP only.

In vitro characterization of purified calcium sensor variants reveal similar dual-emission behavior as for GO-Matryoshka only, with two emission maxima at λₑₘ˜510 nm and λₑₘ˜570 nm upon excitation λ_{exc}˜440 nm or single emission maximum at λₑₘ˜510 nm upon λ_{exc} 488 nm excitation. Titration of the calcium response yielded a large positive response in the green emission channel for all sensors with only minimal response in the orange channel (FIG. 8C). The latter is a result of fluorescence bleed-through from green emission into the orange emission channel, which was estimated to be 10% and was corrected.

Quantitative analysis of the calcium titration revealed different affinities towards calcium (K_{d}=197-501 nM) for the three different MatryoshCaMP variants. MatryoshCaMP yielded a calcium affinity of 197 (±22) nM, which was similar to 175 (±17) nM obtained for the control GCaMP6s. sfMatryoshCaMP had a K_{d} of 501 (±64) nM, similar to the K_{d} of 481 (±45) nM estimated for the control sfGCaMP. For sfMatryoshCaMP-T78H, the K_{d} was 271 (±10) nM, also similar to the K_{d} of 303 (±28) nM for the control sfGCaMP-T78H. The dynamic range (ΔR/R₀) calculated for the ratiometric MatryoshCaMP variants ranged from 7.6 to 9-fold. The lowest value was found for sfMatryoshCaMP (ΔR/R₀=7.6±0.3), followed by MatryoshCaMP (ΔR/R₀=8.5±0.2). The highest value was calculated for sfMatryoshCaMP-T78H (ΔR/R₀=11.9±0.6). Comparison with the individual ΔF/F₀ at λ_{exc} 440 nm, consistent values were obtained (Table 4). However, ΔF/F₀ at Δ_{exc} 485 nm yielded a much larger dynamic range. The largest values were obtained for MatryoshCaMP (ΔF/F₀=41.8±0.9) and the control GCaMP6s (ΔF/F₀=49.7±0.4). Reduced values were found for sfMatryoshCaMP (ΔF/F₀=9.1±0.4) and sfGCaMP (ΔF/F₀=12.7±0.92) as well as sfMatryoshCaMP-T78H (ΔF/F₀=16.4±0.8) and sfGCaMP-T78H (ΔF/F₀=19.3±0.2.8).

Titration of pH for all calcium sensors was performed under saturating and non-saturating calcium conditions. For GCaMP6s and MatryoshCaMP the calcium-free values calculated were pK_{a,apo}˜9.6 and ˜9.5, respectively, and the calcium-saturated values were pK_{a,sat}˜6.1 for both. For sfGCaMP and sfMatryoshCaMP the calcium free values were pK_{a,apo}˜8.3 and ˜8.1, respectively and the calcium-saturated values were pK_{a,sat}˜6.0 for both. The values for sfGCaMP-T78H and sfMatryoshCaMP-T78H under calcium free conditions were pK_{a,apo}˜8.5 and ˜8.3, respectively and under calcium-saturated condition were pK_{a,sat}˜5.8 and 5.7, respectively as summarized in the following Table 4.

TABLE 4
Sensor Properties of GCaMP6s, MatryoshCaMP, sfGCaMP,
sfMatryoshCaMP, sfGCaMP-T78H and sfMatryoshCaMP-T78H

| | | Dynamic | Dynamic | Dynamic | | | |
|---|---|---|---|---|---|---|---|
| | K_{d} | range_{440 exc} | range_{440 exc} | range_{485 exc} | Hill | | |
| Sensor | (nM) | ΔR/R₀ | ΔF/F₀ | ΔF/F₀ | coefficient | pK_{a,} ₐₚₒ | pK_{a,} ₐₚₒ |
| GCaMP6s | 175 ± 17 | | 9.8 ± 0.3 | 49.7 ± 0.4 | 2.52 ± 0.09 | 9.63 ± 0.04 | 6.15 ± 0.03 |
| MatryoshCaMP | 197 ± 23 | 8.5 ± 0.2 | 8.6 ± 0.1 | 41.8 ± 0.9 | 2.62 ± 0.05 | 9.49 ± 0.04 | 6.08 ± 0.02 |
| sfGCaMP | 481 ± 45 | | 9.5 ± 1.1 | 12.7 ± 0.2 | 1.83 ± 0.17 | 8.26 ± 0.01 | 6.02 ± 0.02 |
| sfMatryoshCaMP | 501 ± 64 | 7.6 ± 0.3 | 8.1 ± 0.5 | 9.1 ± 0.4 | 2.09 ± 0.11 | 8.14 ± 0.01 | 6.03 ± 0.22 |
| sfGCaMP-T78H | 303 ± 28 | | 11.4 ± 0.5 | 19.3 ± 2.8 | 2.24 ± 0.14 | 8.47 ± 0.01 | 5.78 ± 0.02 |
| sfMatryoshCaMP-T78H | 271 ± 10 | 11.9 ± 0.6 | 12.1 ± 0.5 | 16.4 ± 0.8 | 2.11 ± 0.07 | 8.34 ± 0.01 | 5.74 ± 0.02 |

### Conversion of an Ammonium Activity State Sensor into a Ratiometric Sensor Using the “Matryoshka” Approach

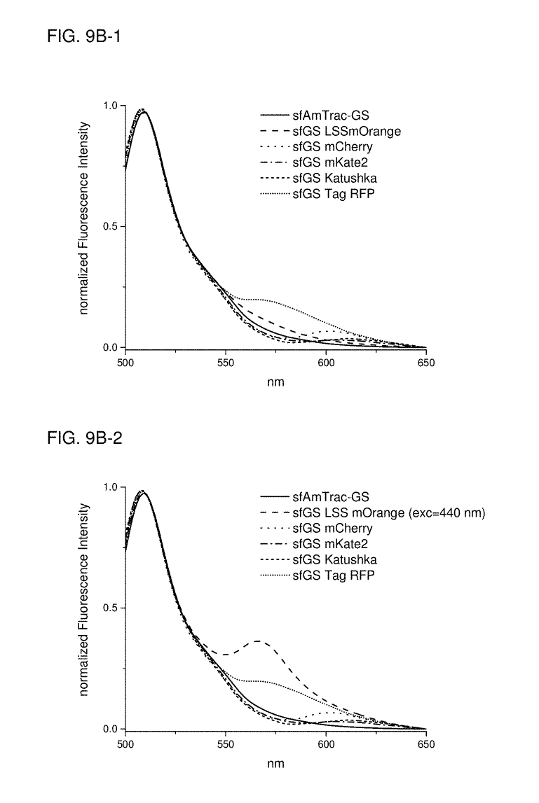

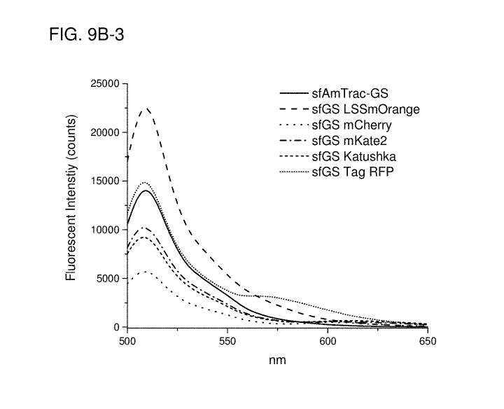

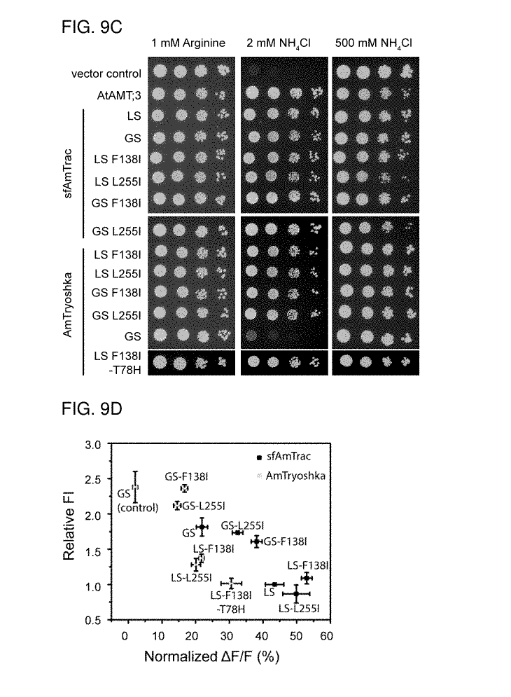

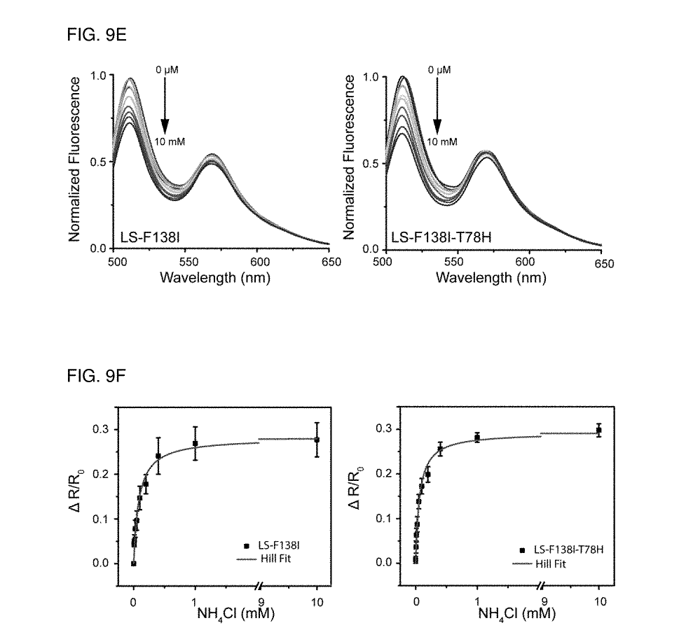

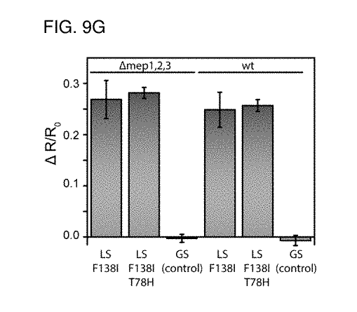

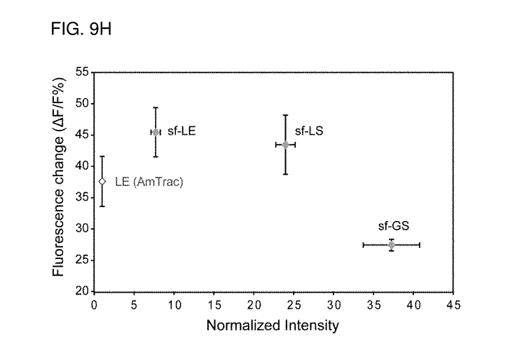

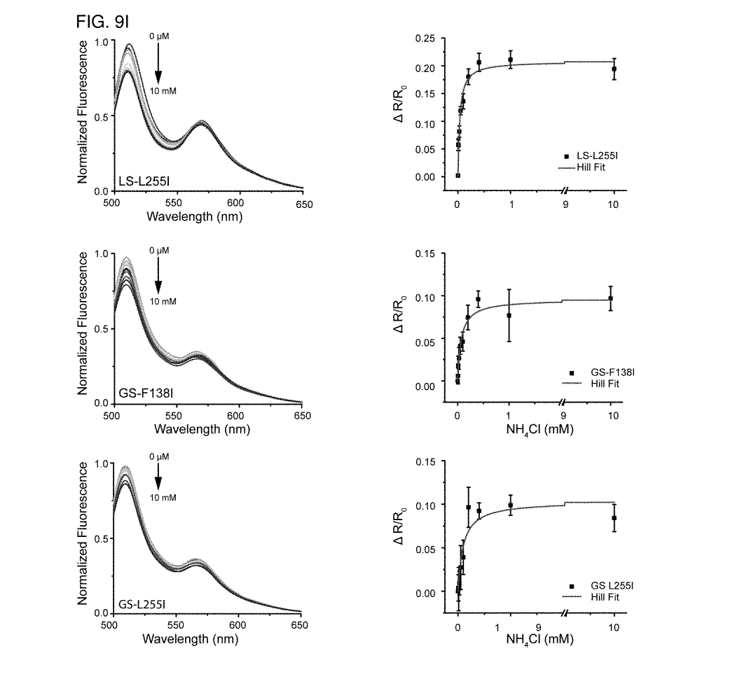

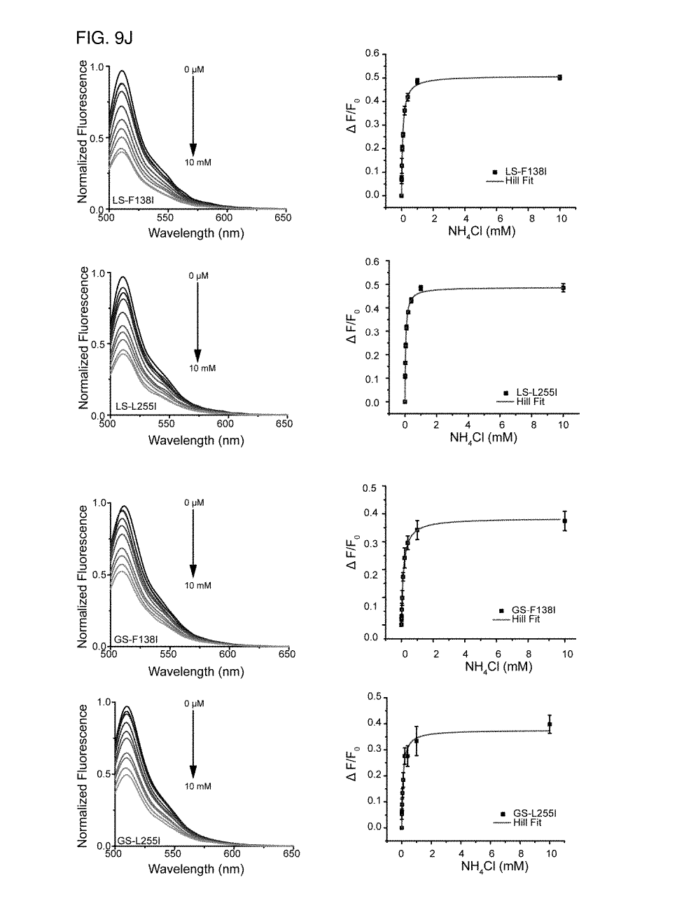

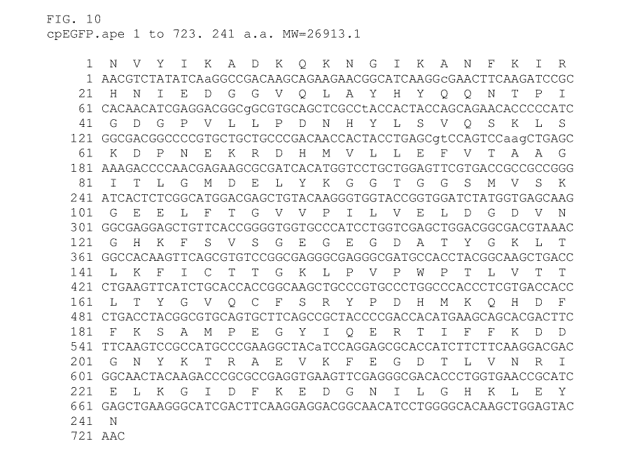

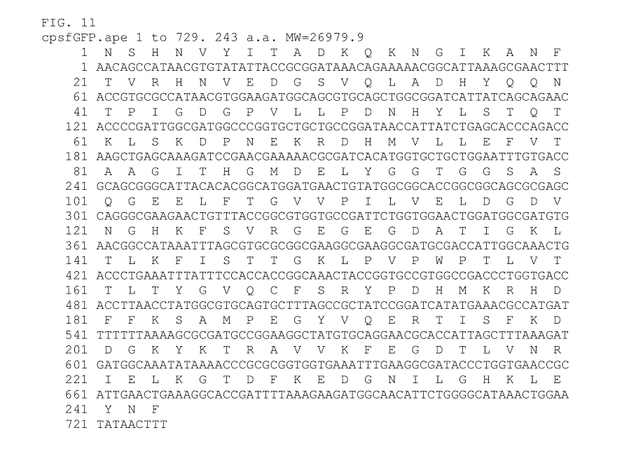

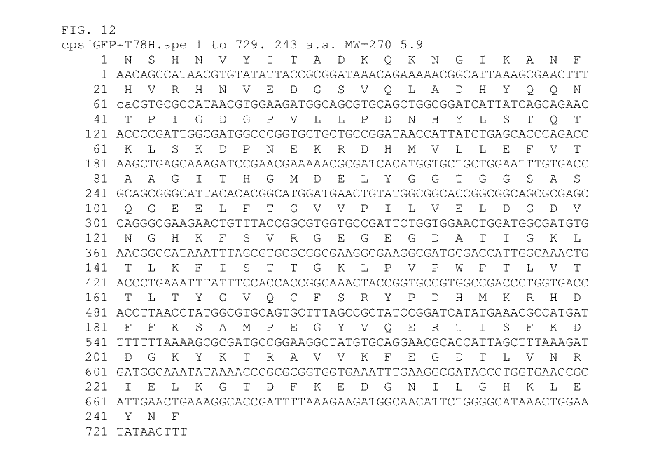

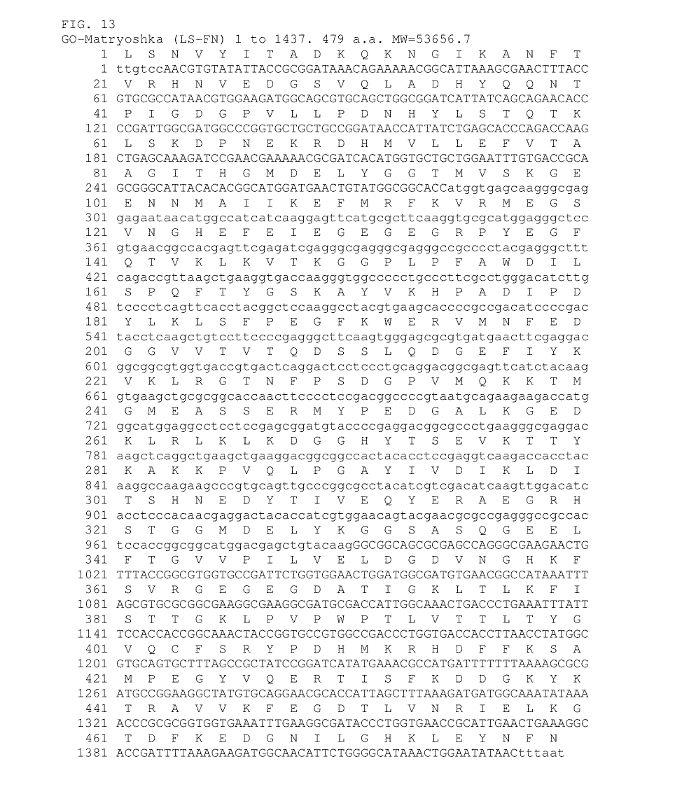

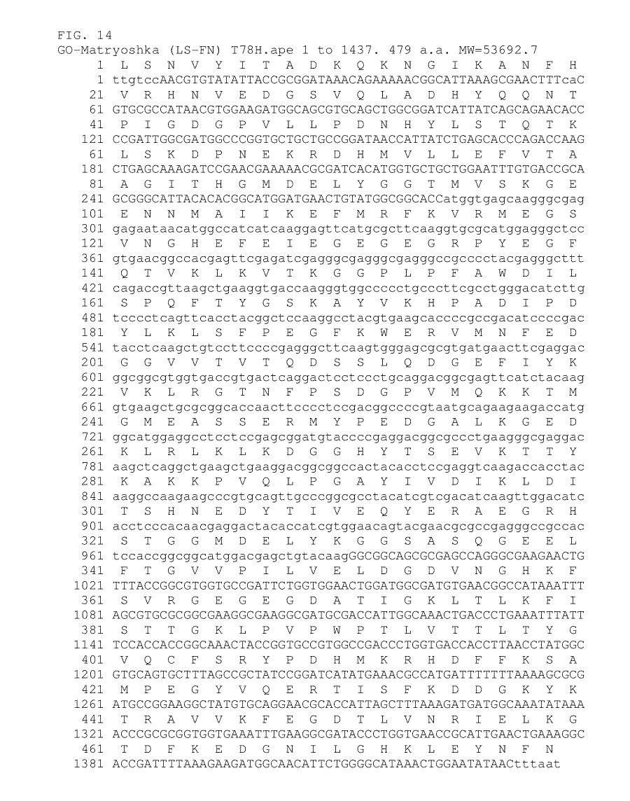

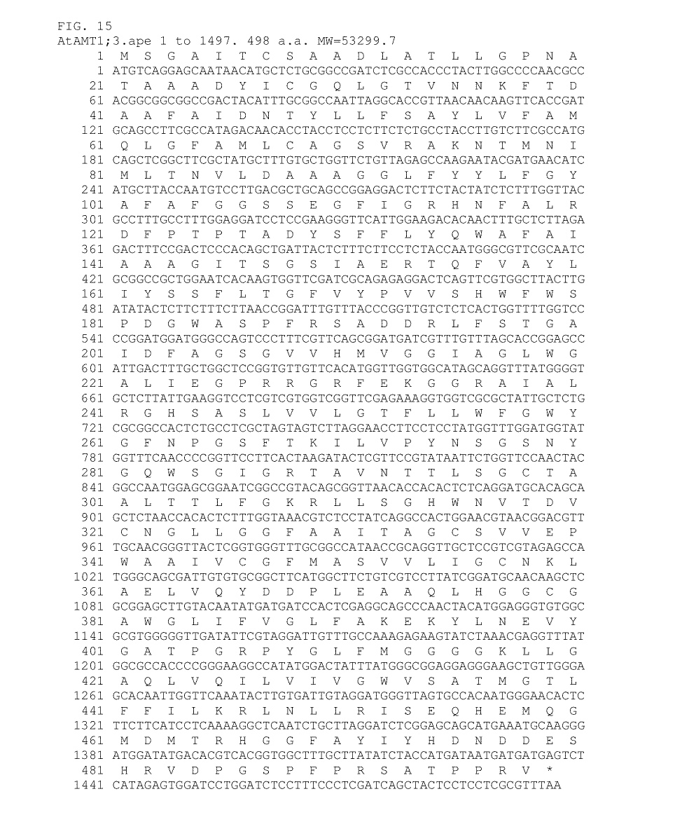

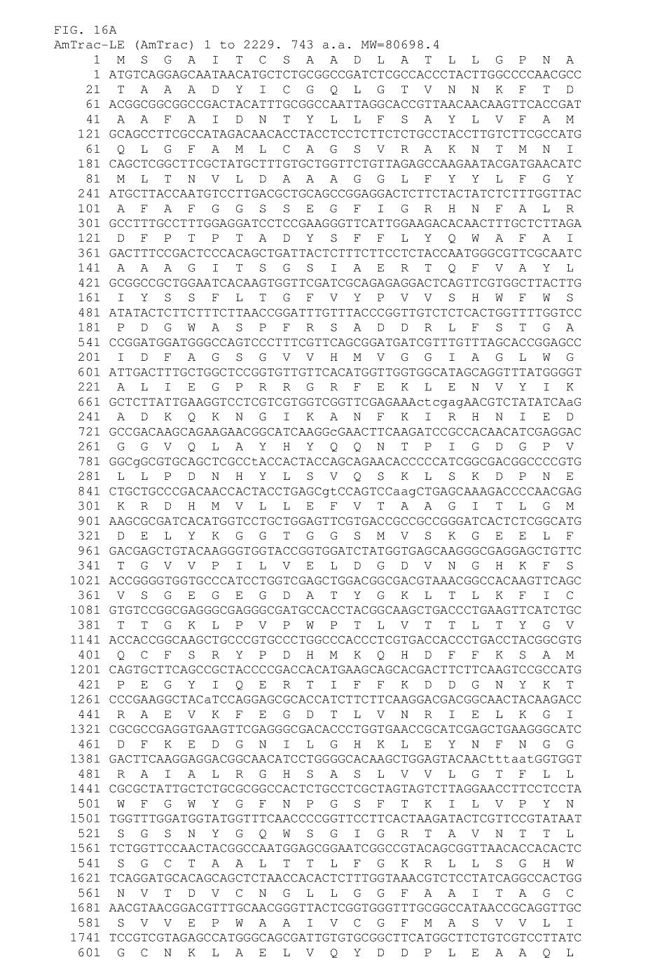

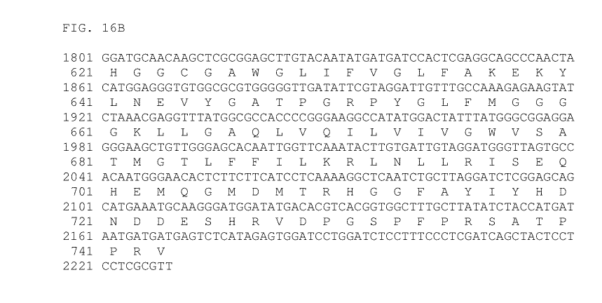

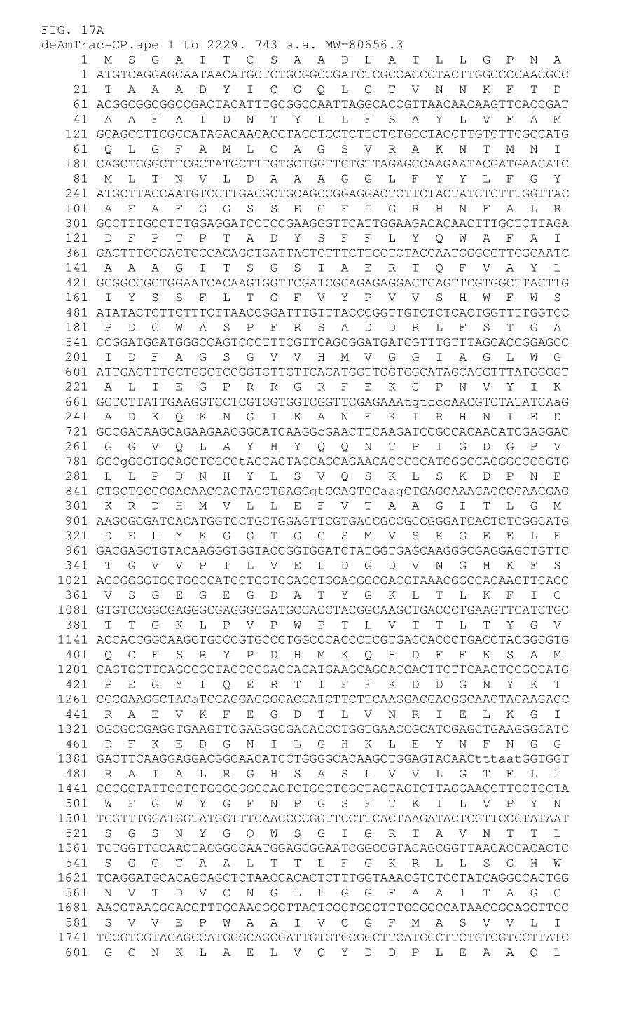

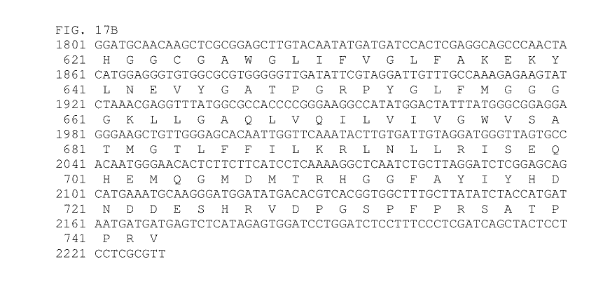

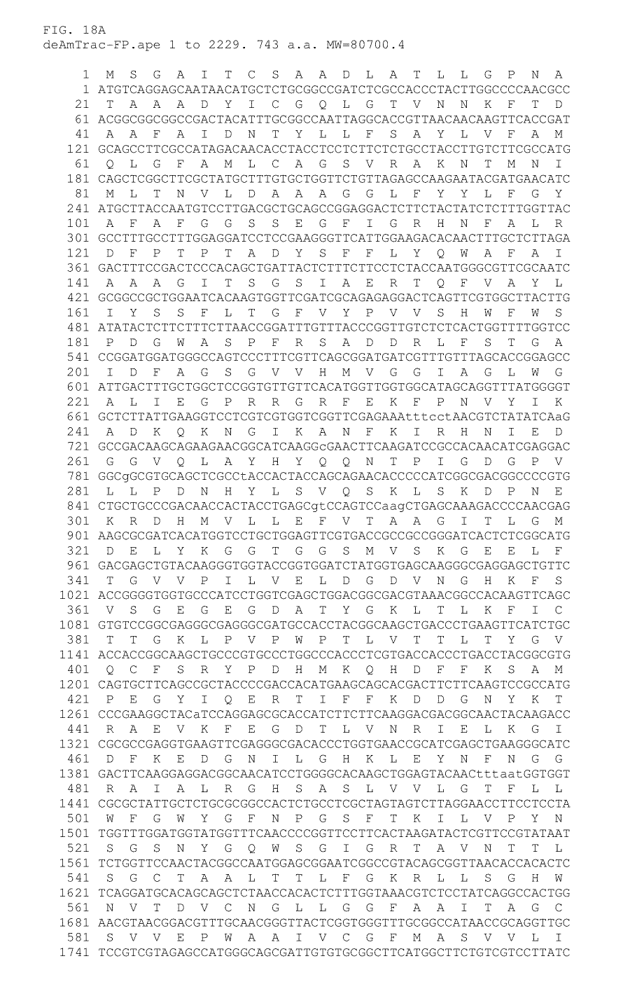

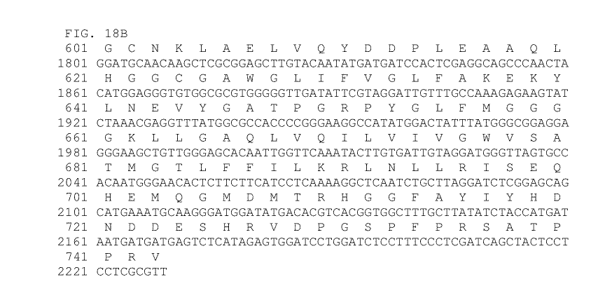

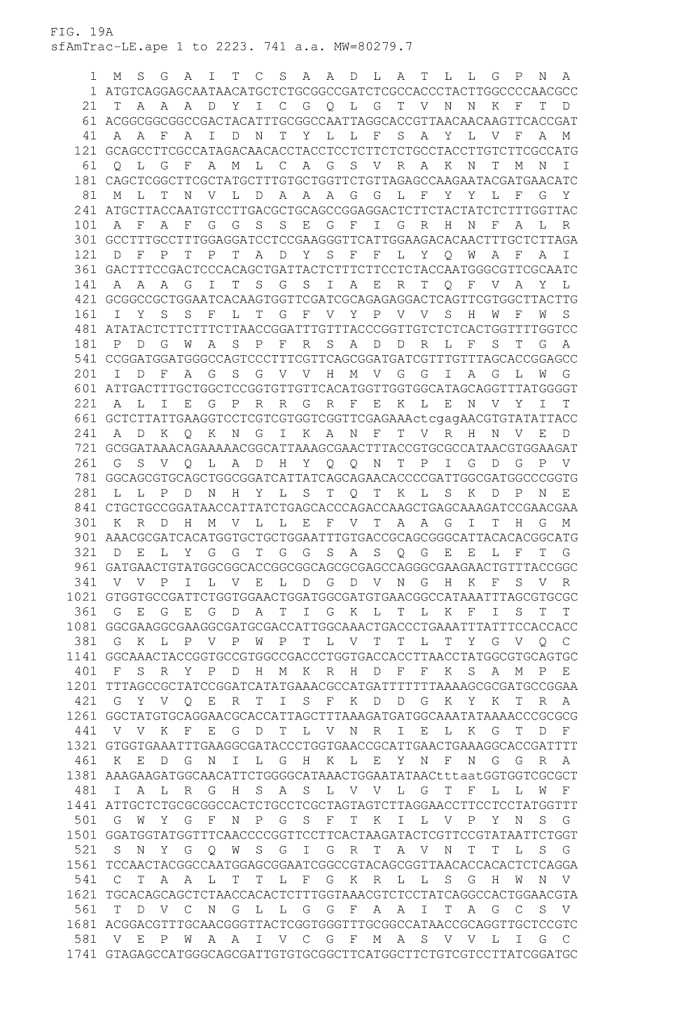

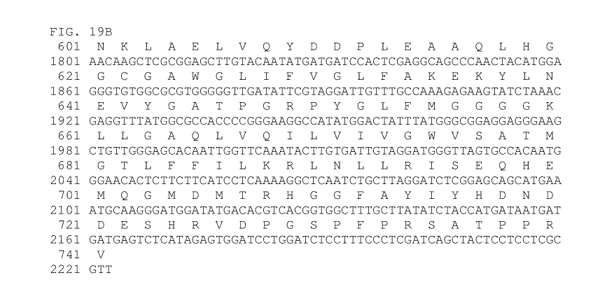

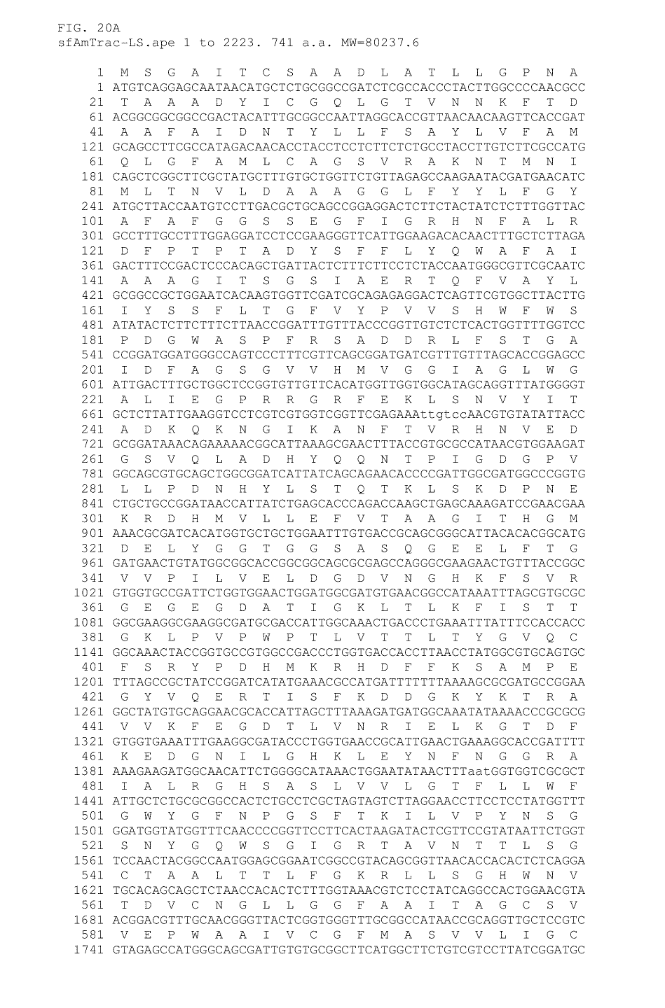

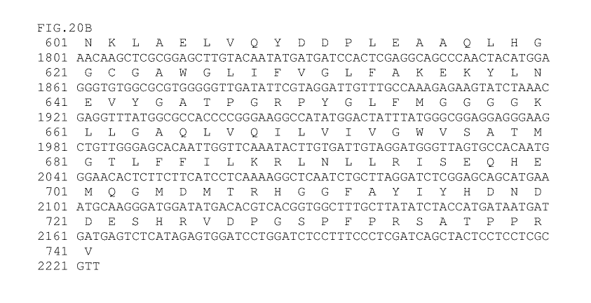

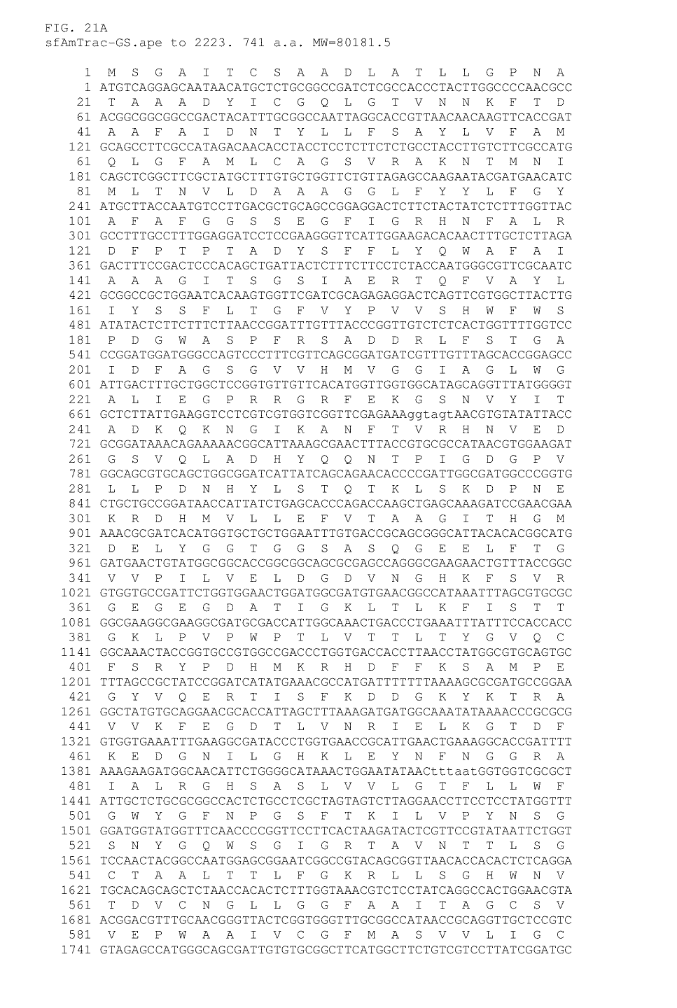

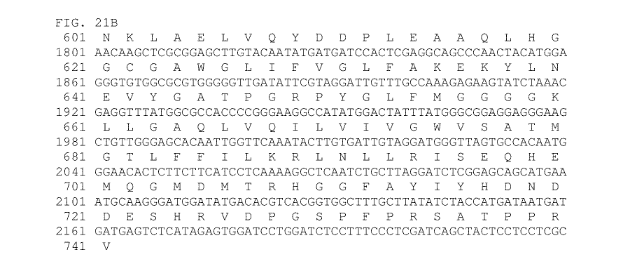

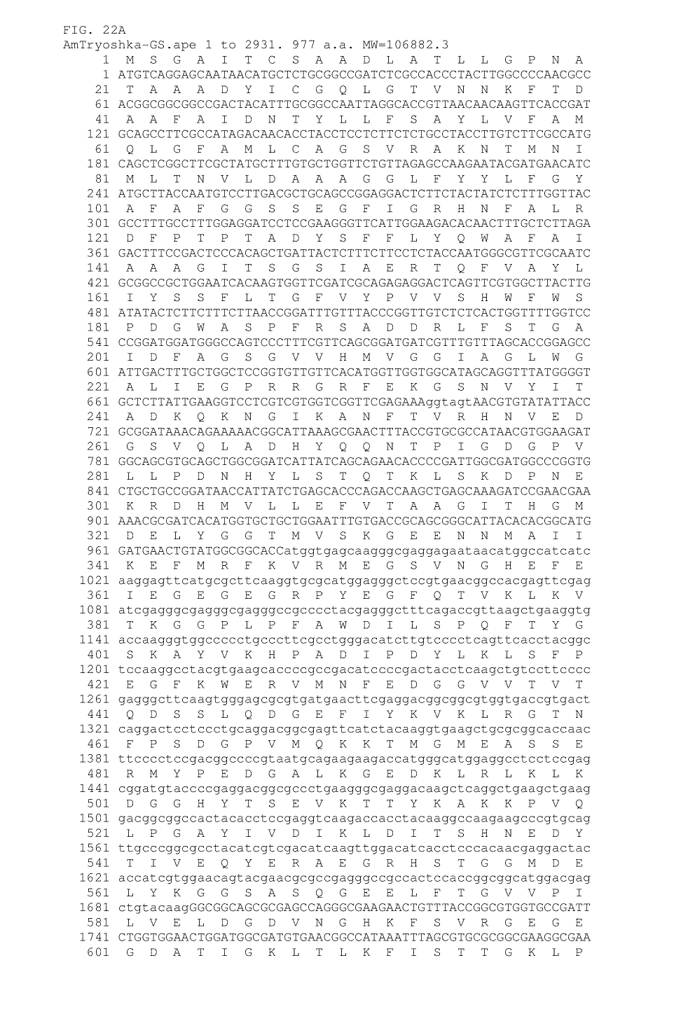

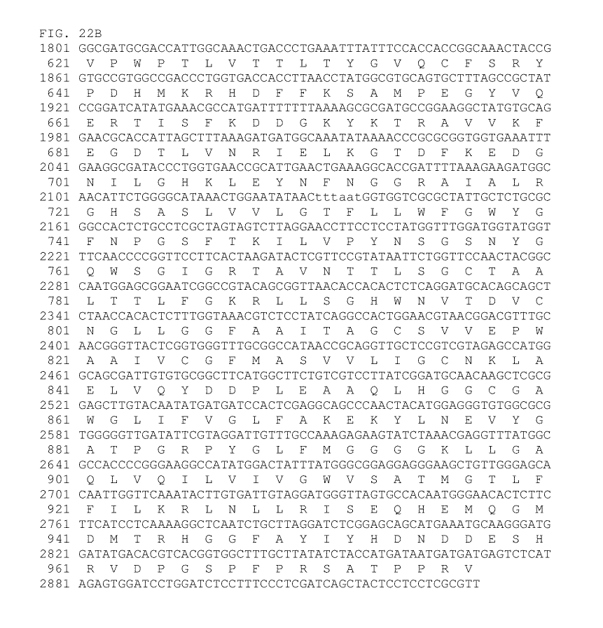

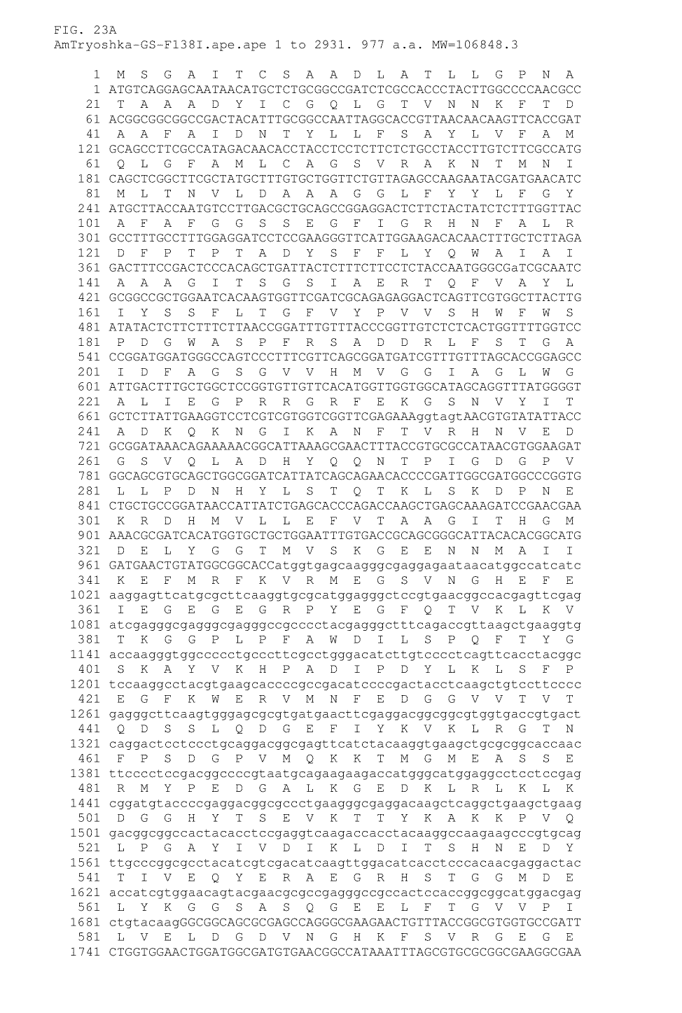

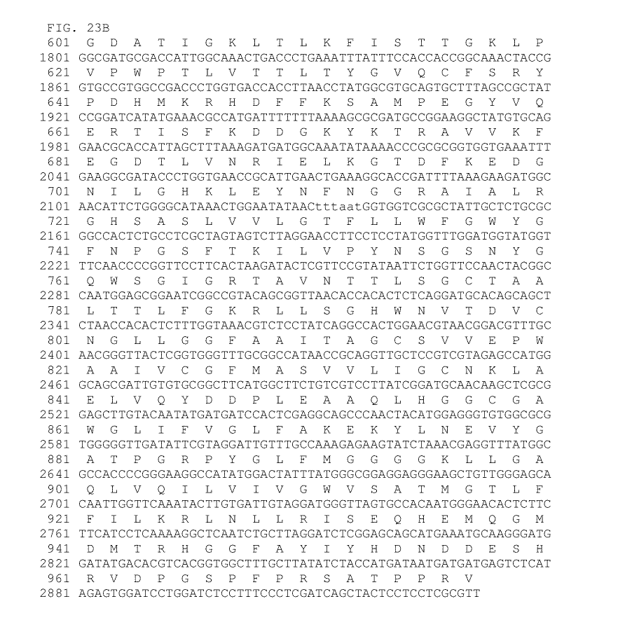

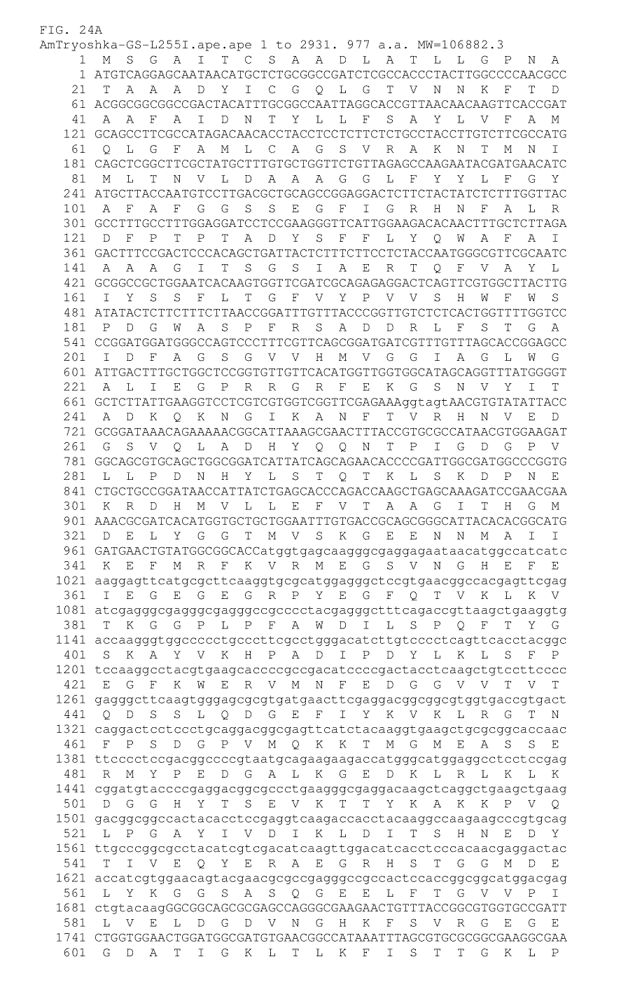

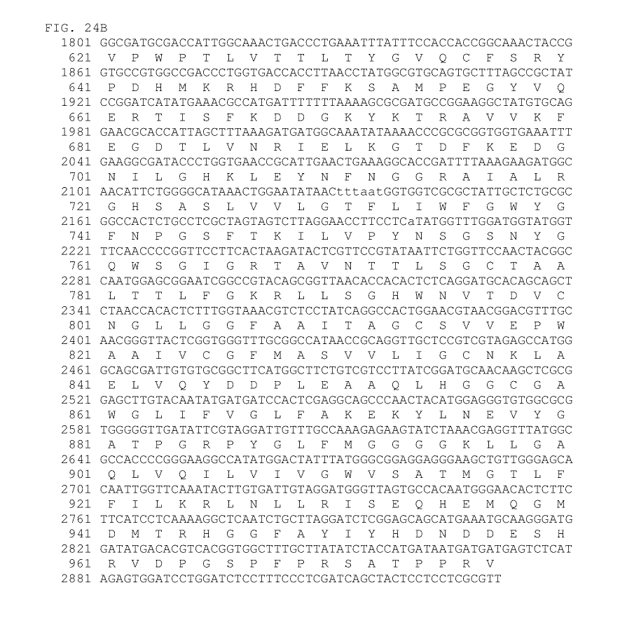

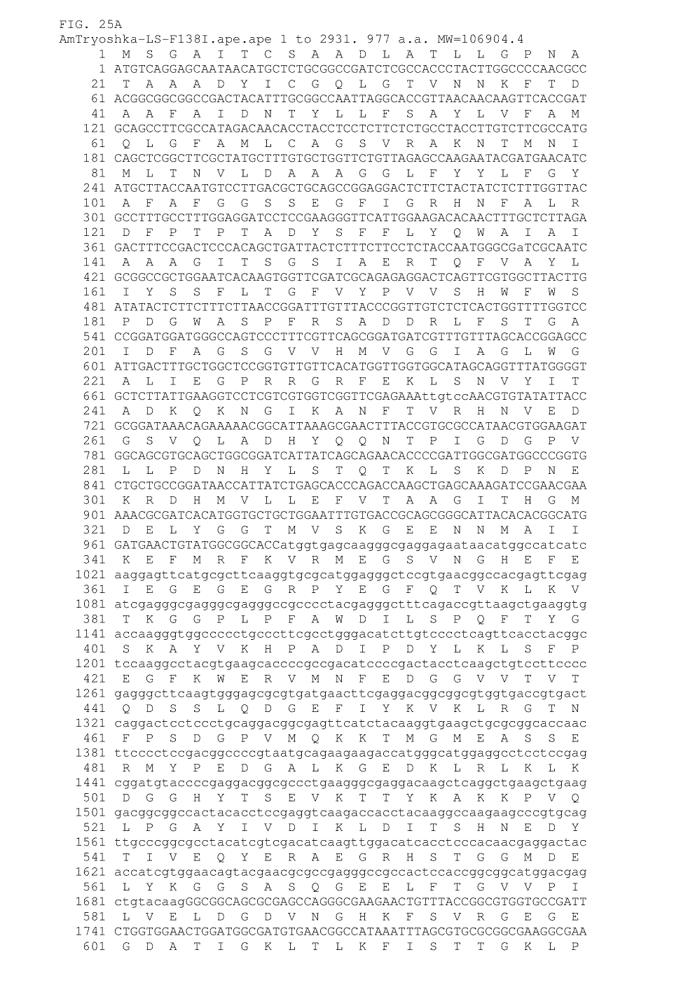

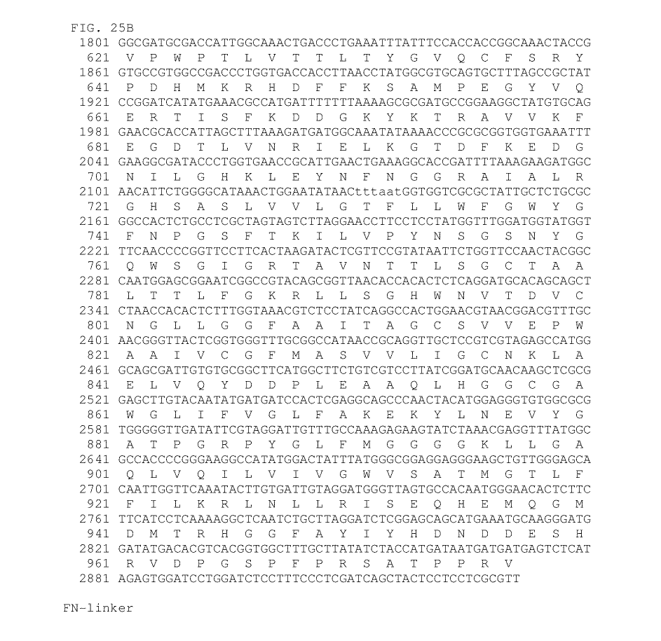

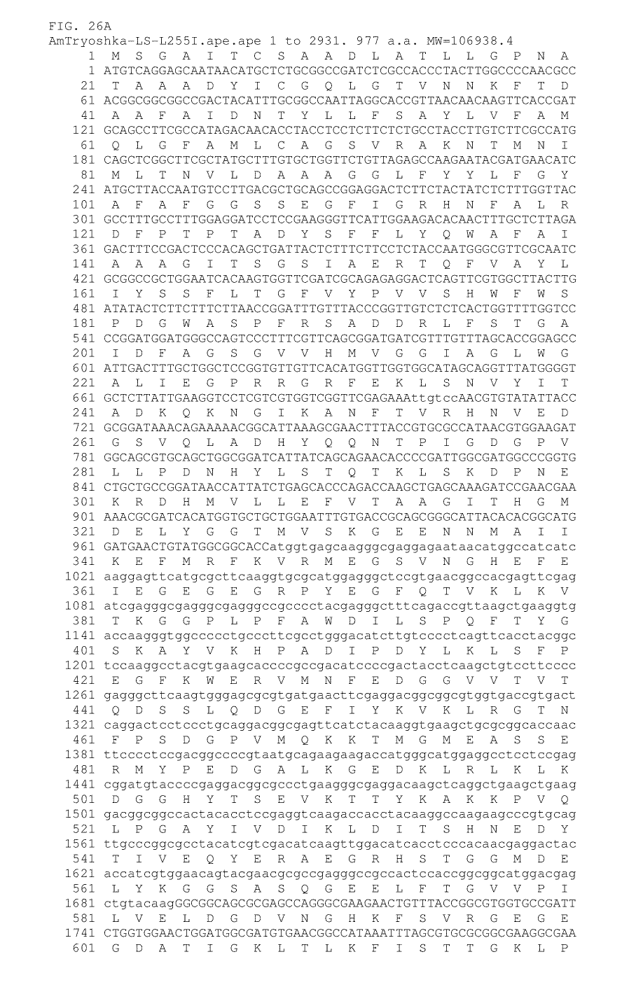

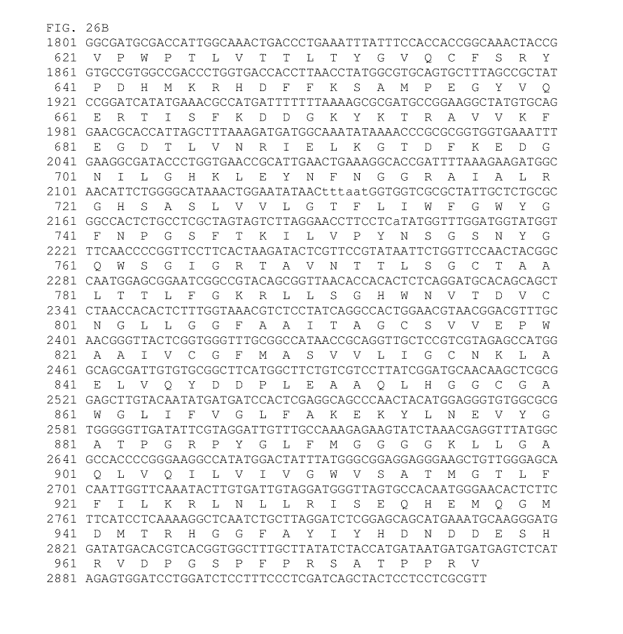

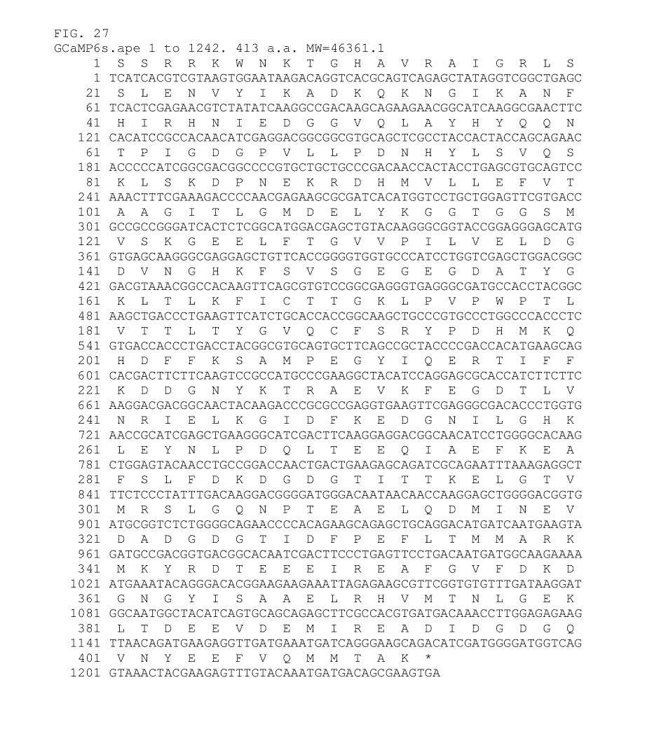

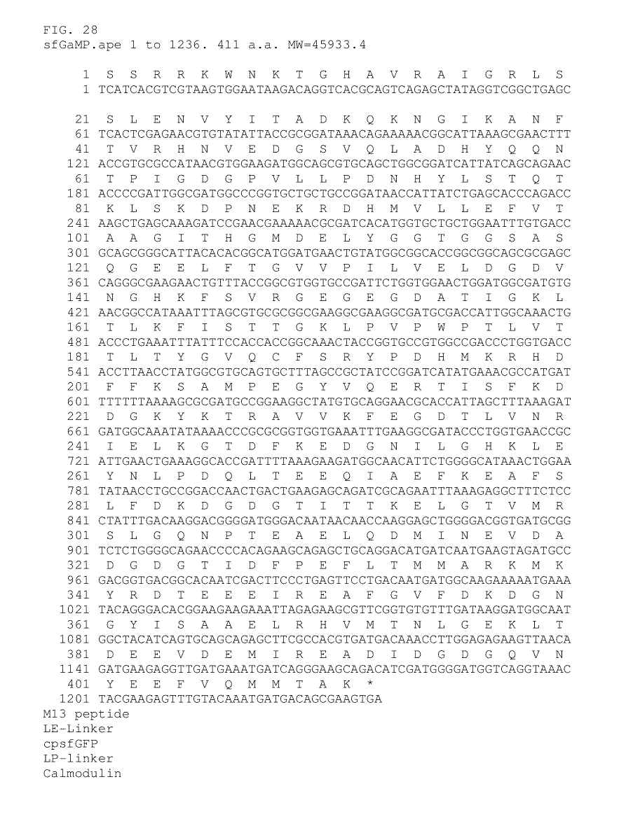

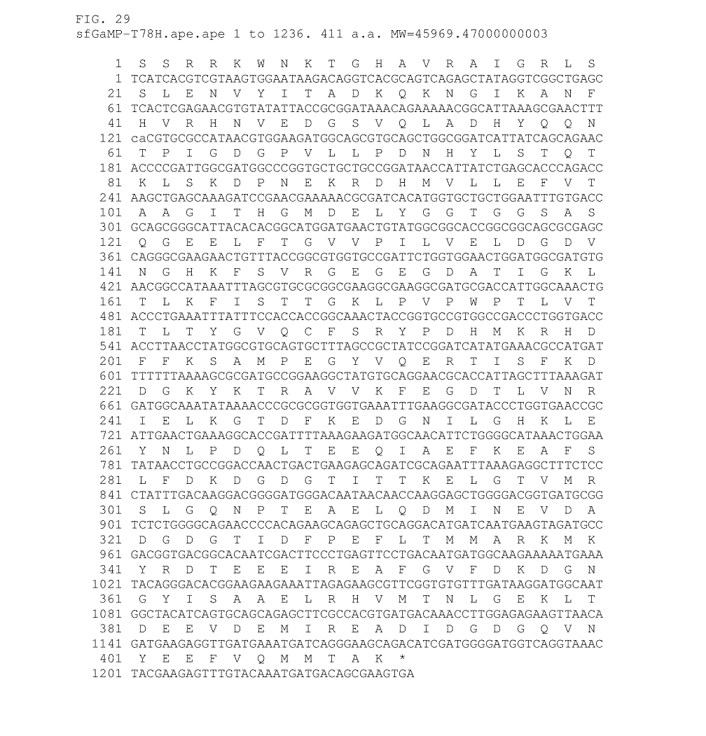

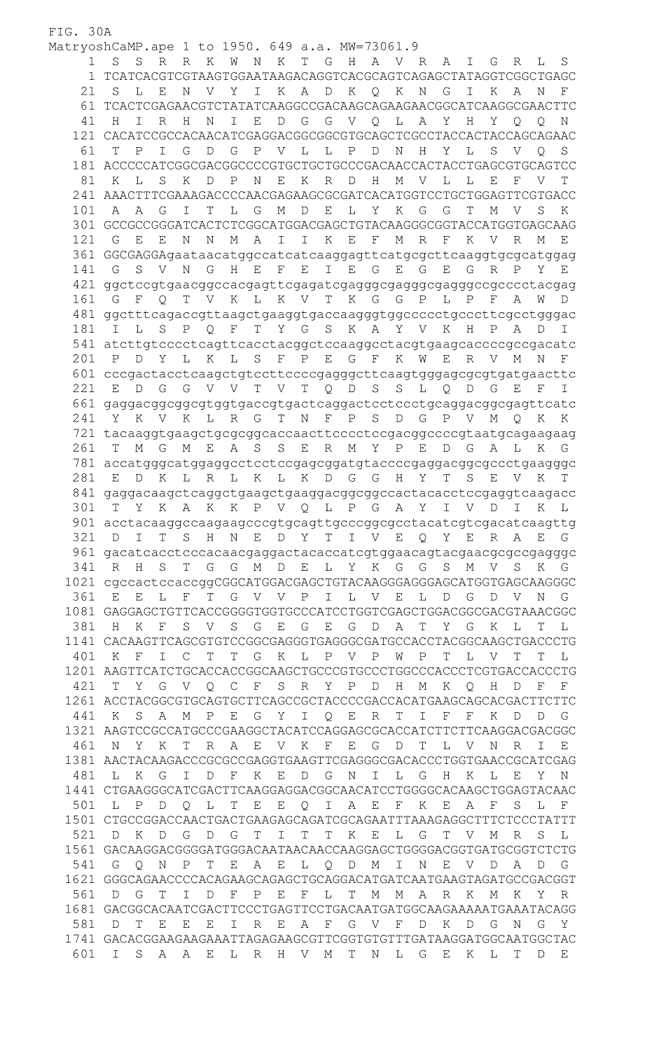

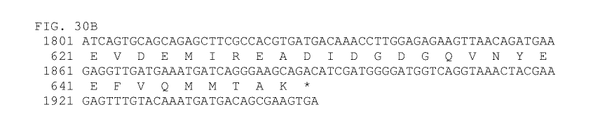

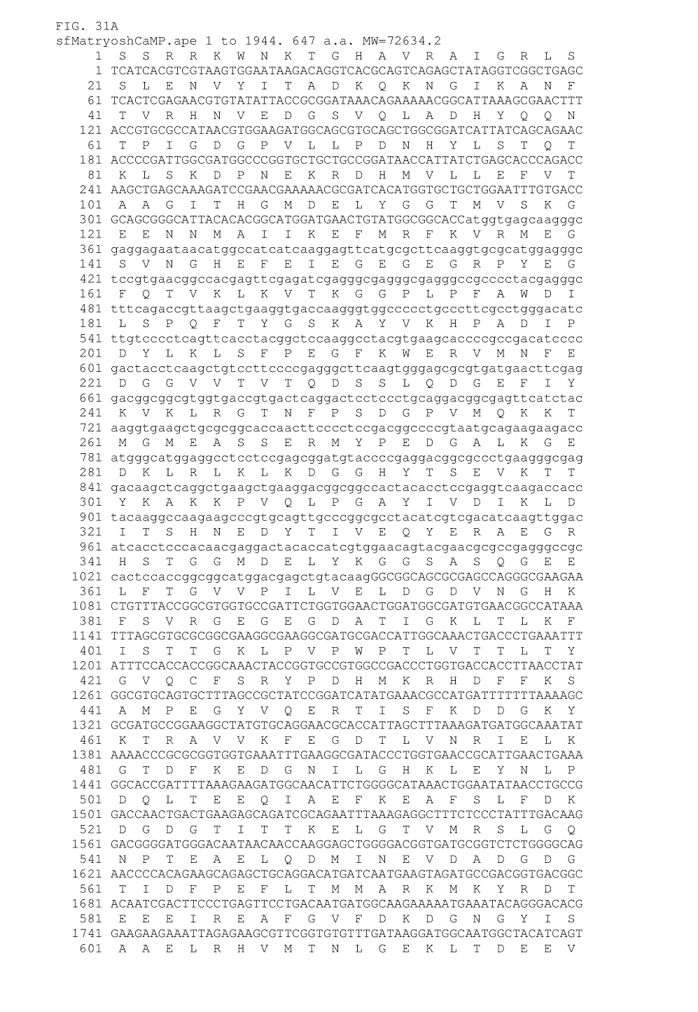

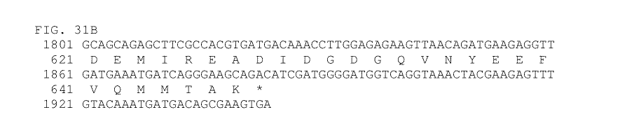

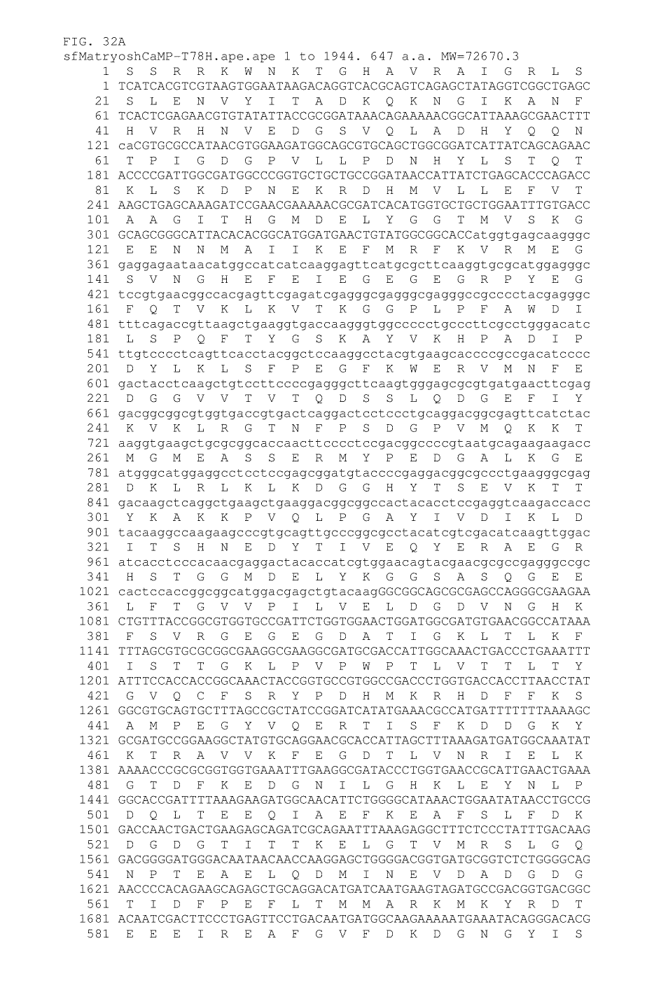

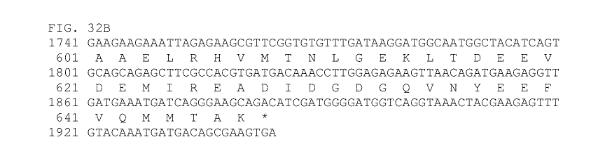

To demonstrate the broad utility of GO-Matryoshka for ratiometric biosensor design, a membrane transporter-based biosensor, termed AmTrac, was employed to enable accurate measurements of ammonium transport activity in vivo. AmTrac was converted into a ratiometric sensor by replacing the cpEGFP with GO-Matryoshka (FIG. 9A).

As controls, the cpEGFP was substituted with the cpsfGFP only, generating sfAmTracs. The circular permutation breakpoint of the cpsfGFP was modified according to the linker compositions reported for AmTrac-LS and -GS, with the left linker being glycine and serine (GS) or leucine and serine (LS) and the right linker being phenylalanine and arginine (FN). Yeast transformed with the resulting AmTryoshka variants showed bright green and orange fluorescence intensity (FI) at λ_{exc} 440 nm but no detectable response upon ammonium treatment. Ammonium transporters are sensitive membrane proteins and their activity is easily affected by manipulation of their sequence. Accordingly, insertion of GO-Matryoshka impaired transport activity, as shown by the growth complementation assay of the AMT-deficient yeast mutant on low ammonium medium (FIG. 9C bottom row). sfAmTrac-GS and -LS showed an increase in basal fluorescence intensity (FI) of 36-fold and 24-fold, respectively, compared to AmTrac-LE (FIG. 9H) and a response to ammonium addition of about 25% and 40% fluorescence intensity (FI) decrease, respectively, as compared to 37% of AmTrac-LE (FIG. 9D and FIG. 9H).

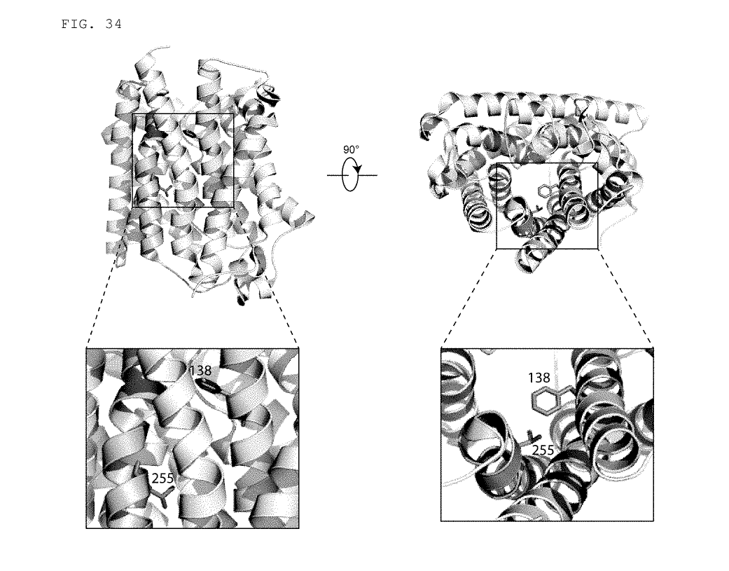

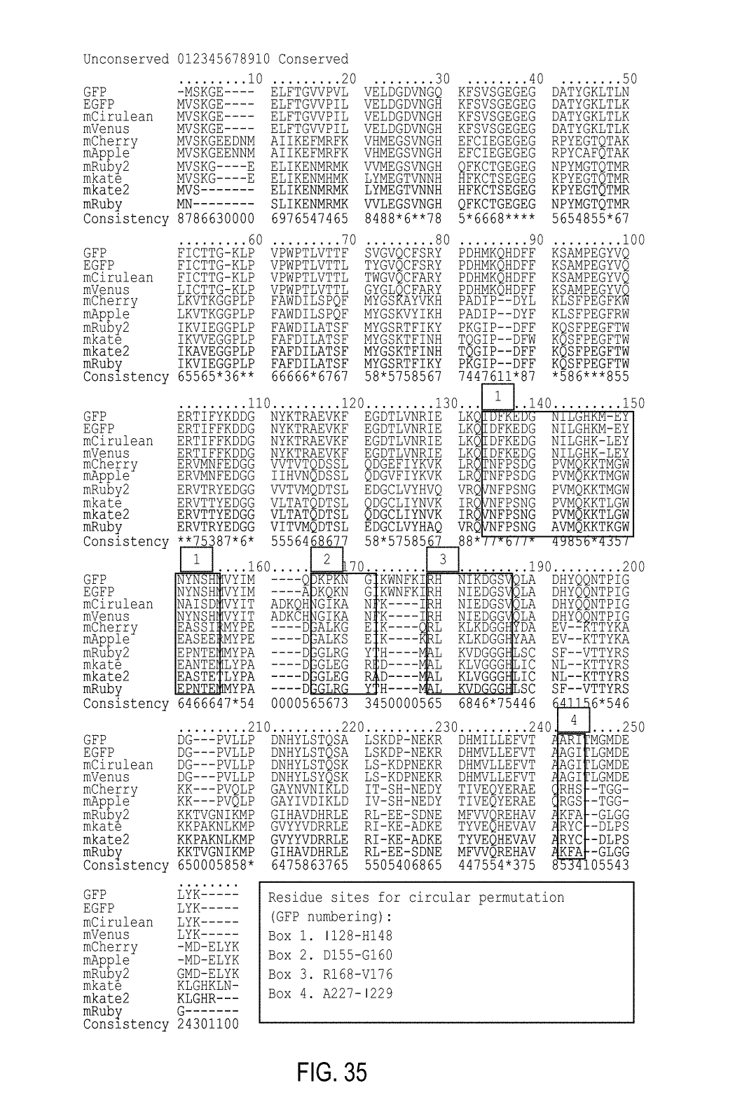

A suppressor screen using the inactive AmTryoshka was performed to identify suppressor mutants that would restore the ammonium transporter activity. Two individual mutations, F138I and L255I, were identified that allowed for growth on low ammonium medium (FIG. 9C). The mutations were termed according to the AtAMT1;3 residue numbers. The crystal structure of AfAMT1 (PDB: 2B2F) served as representation to illustrate that both residues F and L are pointing towards the inside of the pore of the AMT, a position that easily justifies the recovery of the transport activity (data not shown FIG. 34).

Steady state analysis of ammonium titrations of yeast expressing AmTryoshka-F138I and -L255I revealed a reduction in fluorescence intensity (FI) in the green channel by 25-30% for the LS-linker variant and 10-15% for the GS-linker variant (FIGS. 9D, 9E and 9I). Quantitative analysis of the ratio (ΔR/R₀) as response to ammonium titration was performed (FIGS. 9E and 91). The obtained affinity constants (Table 5) were similar to the value obtained for AmTrac (Kₘ˜0.55 μM)¹². A fluorescence bleed-through factor of 8%, calculated from green emission in the orange emission channel, was included in the ratio analysis.

TABLE 5
AmTryoshka affinity constants

| Sensor | Affinity constant [μM] |
|---|---|
| sfAmTrac-GS | 51.8 ± 4.4 |
| sfAmTrac-LS | 58.3 ± 15.1 |
| sfAmTrac-GS-F138I | 127.9 ± 20.6 |
| sfAmTrac-LS-F138I | 81.1 ± 7.1 |
| sfAmTrac-GS-L255I | 117.7 ± 28.5 |
| sfAmTrac-LS-L255I | 48.1 ± 3.8 |
| AmTryoshka-GS-F138I | 68.9 ± 26.7 |
| AmTryoshka-LS-F138I | 84.6 ± 20.4 |
| AmTryoshka-GS-L255I | 98.8 ± 45.7 |
| AmTryoshka-LS-255I | 35.0 ± 5.9 |
| AmTryoshka-LS-F138I-T78H | 62.4 ± 8.6 |

The effects of the mutations F138I and L255I in the sfAmTrac-LS and -GS backgrounds, containing cpsfGFP as the single FP without reference domain were also analyzed. A fluorescence intensity (FI) change of ˜20% and ˜40% upon ammonium treatment was found for AmTrac-GS and -LS, respectively. Each individual point mutation increased the response to ˜50%. (FIGS. 9D and 9J).

To exclude environmental effects, such as accumulation of intracellular ammonium, which in turn could vary the pH, wild type yeast expressing its functional ammonium transporters was transformed with AmTryoshka-LS-F138I, -T78H or the non-responsive control AmTryoshka-GS. The responses in the wild type background were similar to those in the AMT-deficient mutant (20-30% for the responding transporters and no response for the negative control), indicating that intracellular ammonium levels did not affect the fluorescent intensity (FI) and thus sensor response (FIG. 9G).

DNA Constructs

For the generation of sfAmTrac-LS and GS, overlap-PCR was employed to exchange the cpEGFP for the cpsfGFP. Briefly, three DNA fragments were generated, the N-terminal AtAMT1;3 fragment (amino acid 1-233), the C-terminal AtAMT1;3 fragment (amino acid 234-498) and the cpsfGFP fragment.

cpsfGFP was amplified from the pET15b-cpsfGFP with the forward primer AmLS_sfGFPcp_FW (SEQ ID NO:133) including the coding sequence for the LS linkers and GS linkers AmGS_sfGFPcp_FW (SEQ ID NO:134), respectively, to replace the NSH linker on the N-terminus of the cpsfGFP and the reverse primer coding for FN AmFN_sfGFPcp_RV (SEQ ID NO:135) to replace the F linker on the C-terminus of cpsfGFP sequence. Thus, the cpsfGFP contains the equivalent breakpoint in the sfAmTracs, as the original AmTracs12. The fragments were combined into the pDONR-221 vector via Gateway BP-reaction and then moved into pDRF′-GW via Gateway LR reaction (Invitrogen Life Technology, Paisley, United Kingdom).

The sfAmTrac-GS-LSSmOrange sequence was synthesized using GeneScript and introduced into pDRF′-GW vector via Gateway reaction (Invitrogen Life Technology, Paisley, United Kingdom). pDRF′-sfAmTrac-GS-LSSmOrange served as base for the AmTryoshka generation (see yeast transformation and culture).

AmTryoshka-LS-F138I and -L255I as well as sfAmTrac-GS-F138I/L255I and sfAmTrac-LS-F138I/L255I were generated via site-directed mutagenesis performed according to the guidelines of the QuikChange II XL Site-Directed Mutagenesis Kit (Stratagene, Agilent Technologies, Santa Clara, USA). Primers sfLS-LSSmO_FW (SEQ ID NO:136) and sfLS-LSSmO_RV (SEQ ID NO:137) exchanged the GS sequence for LS, primers sfAmTrac-F138I_FW (SEQ ID NO:138) and sfAmTrac-F138I_RV (SEQ ID NO:139) introduced the F138I mutation and primers sfAmTrac-L255I_FW (SEQ ID NO:140) and sfAmTrac-L255I_RV (SEQ ID NO:141) introduced the L255I mutation, respectively.

pET15b cpsfGFP-GS-FN and pET15b cpsfGFP-LS-FN for in vitro characterization were generated by modifying the circular permutation breakpoint of cpsfGFP sequence in the bacterial expression vector pET15b via site-directed mutagenesis. Primer pairs GS-cpsfGFP_FW/GS-cpsfGFP_RV (SEQ ID NOS: 144 and 145) and LS-cpsfGFP_FW/LS-cpsfGFP_RV (SEQ ID NOS: 142 and 143) were used to replace the NSH sequence for GS and LS, respectively and primer pair cpsfGFP-FN_FW/cpsfGFP-FN_RV (SEQ ID NOS: 146 and 147) replaced the F with FN.

sfGO-Matryoshka variants were created by digesting the pET-15b cpsfGFP plasmid and the pDRF′-sfAmTrac-GS-LSSmOrange construct with AgeI-HF and DraIII-HF (New England Biolabs, Ipswich, Mass.), gel-purification with a commercial kit (Machery-Nagel, Düren, Germany), and ligation by T4 DNA ligase (Thermo Scientific) subsequently inserting the LSSmOrange into the center of the cpsfGFP, GGT-GGS (SEQ ID NO:112) flexible linker, creating pET15b-sfGO-Matryoshka GS-FN and pET15b-sfGO-Matryoshka-LS-FN, respectively.

pET15b-LSSmOrange construct was generated by an initial PCR amplification of the LSSmOrange sequence using the primers LSSmOr-pET15b_InF_1st_FW (SEQ ID NO:148) containing a HIS tag overhang and LSSmOr-pET15b_InF_1st_RV (SEQ ID NO:149) adding a stop codon. A second round of PCR amplification with primers LSSmOr-pET15b_InF_2nd_FW (SEQ ID NO:150) and LSSmOr-pET15b_InF_RV (SEQ ID NO:151) was performed to add overlaps for subsequent In-Fusion® HD cloning (Clontech). pET-15b cpsfGFP was digested with XhoI and NcoI-HF (New England Biolabs) to remove the cpsfGFP and In-Fusion® cloning was performed per Clontech's protocol to recombine the purified fragments.

Calcium sensor variants were cloned by digesting the full calcium sensor sequence out of pGP-CMV-GCaMP6s8 (Addgene plasmid #40753) with MfeI and NheI-HF and ligated into the bacterial expression vector pRSETa linearized with NheI-HF and EcoRI-HF. pRSETa MatryoshCaMP6s was produced by inserting a PCR amplified LSSmOrange into the middle of the GGT-GGS (SEQ ID NO:112) flexible linker of the KpnI digested pRSETa GCaMP6s construct via In-Fusion® (GCaMP6-EGFPcp-LSSmO-InF_FW (SEQ ID NO:152) and GCaMP6-EGFPcp-LSSmO-InF_RV (SEQ ID NO:153)). pRSETa sfGCaMP6s and pRSETa sfMatryoshCaMP6s were assembled by substituting the cpEGFP of GCaMP6s with either a cpsfGFP or a sfGO-Matryoshka. The full length sequences of cpsfGFP and sfGO-Matryoshka, respectively, were PCR amplified with overlaps containing 9 bp of the 3′ end of the M13 peptide and XhoI restriction site/LE amino acid linker as well as the C-terminal LP amino acid linker and 14 bp of the 5′ end of the calmodulin protein (sfGFPcp-XhoI-M13-InF_FW (SEQ ID NO:154) and sfGFPcp-LP-CaM_RV (SEQ ID NO:155)). Another PCR fragment was generated with the full GCaMP6s calmodulin protein containing 21 bps of overlap with the cpsfGFP 3′ end (CaM-LP-sfGFPcp_FW (SEQ ID NO:156) and CaM-pRSET-HindIII-InF_RV (SEQ ID NO:157)). The two fragments were then ligated via a two-step PCR protocol and the resulting PCR product was recombined by In-Fusion® into pRSETa GCaMP6s that had been digested with XhoI and HindIII-HF.

Yeast Transformation and Culture

The in vivo measurements employed the yeast strain 31019b [mep1Δ mep2Δ::LEU2 mep3L:KanMX2 ura3]²⁸, which lacks all endogenous MEP ammonium transporters^{27,28}. Briefly, yeast transformation was performed using the lithium acetate protocol²⁹. Transformants were plated on solid YNB (minimal yeast medium without amino acids/ammonium sulfate; Difco BD, Franklin Lakes, N.J.) supplemented with 3% glucose and 1 mM arginine. Single colonies were selected and inoculated in 5 ml liquid YNB supplemented with 3% glucose and 0.1% proline under agitation (230 rpm) at 30° C. until OD600 nm 0.5-0.9.

sfAmTrac-GS-LSSmOrange, which did not show a response upon ammonium treatment, was subjected to a suppressor screen. Here, liquid cultures were washed twice with sterile water, the final resuspension volume being 5 mL and 500 μL were streaked on five plates with a diameter of 150 mm (VWR, Radnor, Pa., USA) of solid YNB medium buffered with 50 mM MES/Tris, pH 5.2, supplemented with 3% glucose and 1 mM NH₄Cl. The plates were incubated at 30° C. and single colonies were identified after 7 days. Yeast plasmid DNA was isolated and sequenced, revealing the mutations F138I and L255I. The sfAmTrac-GS-LSSmOrange including the mutations was called AmTryoshka-GS-F138I and -L255I.

For the complementation assay, liquid cultures were diluted 10⁻¹, 10⁻², 10⁻³ and 10⁻⁴ in water and 5 μl of each dilution was spotted on solid YNB medium buffered with 50 mM MES/Tris, pH 5.2, supplemented with 3% glucose and either NH₄Cl (2 mM; 500 mM) or 1 mM arginine as the sole nitrogen source. After 3 days of incubation at 30° C., cell growth was documented by scanning the plates at 300 dpi in grayscale mode.

For fluorescence measurements, liquid yeast cultures were washed twice in 50 mM MES pH 6.0, and resuspended to OD₆₀₀ₙₘ˜0.5 in MES pH 6.0, supplemented with 5% glycerol to delay cell sedimentation²⁷.

Protein Expression and Purification

FP constructs in the bacterial expression vector pET-15b and GCaMP6s variants in pRSETa were transformed into BL21 (DE3) cells. Single colonies were grown in Luria broth containing 50 μg/mL carbenicillin at 20° C., shaking in the dark for 48 h. Cells were then harvested by centrifugation and frozen at −20° C. overnight. Pellets were resuspended in 5 mL buffer (20 mM Tris-HCl pH 8), disrupted via sonication (mode), and centrifuged for 1 hour at 4100 rpm and 4° C. to remove cellular debris. The lysate was filtered through 0.45μ and applied to 2 mL Novagen® HIS-Bind® Resin (cat. #69670 EMD Millipore) charged with 50 mM NiCl₂ in Bio-Rad® gravity columns [product info: cat. #731-1550 BioRad] Columns were washed twice with buffer (20 mM Tris-HCl pH 8) and eluted in 1.5-2 mL 200 mM imidazol in 20 mM Tris-HCl pH 8. Purified protein was then allowed to mature overnight at 4° C. before performing measurements. Eluted protein was quantified in accordance with Thermo Scientific's Coomassie (Bradford) Protein Assay kit (Thermo Scientific, Waltham, Mass., USA).

Fluorometric Analysis

All ammonium titrations were performed on a fluorescence plate reader (Safire; Tecan, Männedorf, Switzerland). 200 μL of washed yeast cells expressing the sfAmTrac and AmTryoshka variants were loaded into black 96-well microplates with clear bottom (Greiner bio-one, Germany). For the titrations, 50 μL of NH₄Cl were added to the cells to a final ammonium concentrations of 10 mM, 1 mM, 400 μM, 200 μM, 100 μM, 50 μM, 25 μM, 12.5 μM, 6.25 μM and water was used for the zero value. Cells were incubated for eight minutes to saturate the response. Steady state fluorescence was recorded in bottom reading mode using 7.5 nm bandwidth and a gain of 100. The fluorescence emission spectra (λ_{exc}=440 or 480 nm) and single point values (λ_{exc}=440 or 485 nm; Δₑₘ=510 or 570 nm) were background subtracted using yeast cells expressing a non-florescent vector control. Correction for bleed through, with a calculated bleed through factor of ˜0.08 for green fluorescence in the orange emission channel was performed ΔR/R₀ calculations (R=FI₅₁₀ₙₘ/FI₅₇₀ₙₘ) and fit of the titration kinetics employing a Hill equation. A minimum of three independent transformants was analyzed.

Calcium titrations were carried out using a fluorescence plate reader (Infinite, M1000 Pro; Tecan, Switzerland) and a commercial Calcium Calibration Buffer Kit #1 (Invitrogen Life Technology, Paisley, United Kingdom). The stock solutions of zero-free calcium buffer (10 mM K2EGTA, 100 mM KCl, 30 mM MOPS pH 7.2) and 39 μM calcium buffer (10 mM CaEGTA, 100 mM KCl, 30 mM MOPS pH 7.2) were mixed according to the manufacturer, yielding 11 different free calcium concentrations. 10 μL of purified protein sample was added to 90 μL of buffer zero-free calcium buffer or 39 μM calcium buffer to yield a final protein concentration of 1-1.5 μM and analyzed in 96-well black flat bottom plates (Greiner Bio-One, Germany). Steady state fluorescence spectra were recorded in bottom reading mode using 5 nm bandwidth and a gain of 80 for both excitation and emission wavelengths (λ_{exc}=440 or 480 nm; λₑₘ=525 or 570 nm). Spectra were background subtracted using a buffer control and values of emission maxima were extracted for dynamic range calculations (ΔF/F₀; ΔR/R₀). Throughout the measurements, the temperatures ranged between 25-35° C. and free calcium calculations were adjusted accordingly³⁰. Correction for bleed through, with a calculated bleed through factor of ˜0.1 of green fluorescence in the orange emission channel was performed prior fit of the titration kinetics by a sigmoidal dose response function. A minimum of three independent protein isolations was analyzed.

All graphs and spectral analyses were performed using Origin Pro 2015 software (OriginLab, Northampton, Mass., USA).

### REFERENCES

1. Frommer, W. B., Davidson, M. W. & Campbell, R. E. Genetically encoded biosensors based on engineered fluorescent proteins. Chem. Soc. Rev. 38, 2833-2841 (2009).
2. Kneen, M., Farinas, J., Li, Y. & Verkman, A. S. Green fluorescent protein as a noninvasive intracellular pH indicator. Biophys. J. 74, 1591-1599 (1998).
3. Baird, G. S., Zacharias, D. A. & Tsien, R. Y. Circular permutation and receptor insertion within green fluorescent proteins. Proc. Natl. Acad. Sci. U.S.A. 96, 11241-11246 (1999).
4. Akerboom, J. et al. Crystal structures of the GCaMP calcium sensor reveal the mechanism of fluorescence signal change and aid rational design. J. Biol. Chem. 284, 6455-6464 (2009).
5. Nagai, T., Sawano, A., Park, E. S. & Miyawaki, A. Circularly permuted green fluorescent proteins engineered to sense Ca2+. Proc. Natl. Acad. Sci. U.S.A 98, 3197-3202 (2001).
6. Nakai, J., Ohkura, M. & Imoto, K. A high signal-to-noise Ca(2+) probe composed of a single green fluorescent protein. Nat. Biotechnol. 19, 137-141 (2001).
7. Zhao, Y. et al. An expanded palette of genetically encoded Ca2+ indicators. Science 333, 1888-1891 (2011).
8. Chen, T.-W. et al. Ultrasensitive fluorescent proteins for imaging neuronal activity. Nature 499, 295-300 (2013).
9. Hoi, H., Matsuda, T., Nagai, T. & Campbell, R. E. Highlightable Ca2+ Indicators for Live Cell Imaging. J. Am. Chem. Soc. 135, 46-49 (2013).
10. Fosque, B. F. et al. Neural circuits. Labeling of active neural circuits in vivo with designed calcium integrators. Science 347, 755-760 (2015).
11. Marvin, J. S., Schreiter, E. R., Echevarria, I. M. & Looger, L. L. A genetically encoded, high-signal-to-noise maltose sensor. Proteins 79, 3025-3036 (2011).
12. De Michele, R. et al. Fluorescent sensors reporting the activity of ammonium transceptors in live cells. eLife 2, e00800 (2013).
13. Hanson, G. T. et al. Green fluorescent protein variants as ratiometric dual emission pH sensors. 1. Structural characterization and preliminary application. Biochemistry 41, 15477-15488 (2002).
14. Ast, C., Michele, R. D., Kumke, M. U. & Frommer, W. B. Single-fluorophore membrane transport activity sensors with dual-emission read-out. eLife e07113 (2015). doi:10.7554/eLife.07113
15. Ding, G. et al. In Vivo Tactile Stimulation-Evoked Responses in Caenorhabditis elegans Amphid Sheath Glia. PLoS ONE 10, e0117114 (2015).
16. Deng, H., Gerencser, A. A. & Jasper, H. Signal integration by Ca2+ regulates intestinal stem-cell activity. Nature 528, 212-217 (2015).
17. Kato, S. et al. Global Brain Dynamics Embed the Motor Command Sequence of Caenorhabditis elegans. Cell 163, 656-669 (2015).
18. Lindenburg, L. & Merkx, M. Engineering genetically encoded FRET sensors. Sensors 14, 11691-11713 (2014).
19. Thestrup, T. et al. Optimized ratiometric calcium sensors for functional in vivo imaging of neurons and T lymphocytes. Nat. Methods 11, 175-182 (2014).
20. Tsien, R. Y. The green fluorescent protein. Annu. Rev. Biochem. 67, 509-544 (1998).
21. Deuschle, K. et al. Construction and optimization of a family of genetically encoded metabolite sensors by semirational protein engineering. Protein Sci. Publ. Protein Soc. 14, 2304-2314 (2005).
22. St-Pierre, F. et al. High-fidelity optical reporting of neuronal electrical activity with an ultrafast fluorescent voltage sensor. Nat. Neurosci. 17, 884-889 (2014).
23. Oltrogge, L. M., Wang, Q. & Boxer, S. G. Ground-State Proton Transfer Kinetics in Green Fluorescent Protein. Biochemistry 53, 5947-5957 (2014).
24. Pédelacq, J.-D., Cabantous, S., Tran, T., Terwilliger, T. C. & Waldo, G. S. Engineering and characterization of a superfolder green fluorescent protein. Nat. Biotechnol. 24, 79-88 (2006).
25. Shcherbakova, D. M., Hink, M. A., Joosen, L., Gadella, T. W. J. & Verkhusha, V. V. An Orange Fluorescent Protein with a Large Stokes Shift for Single-Excitation Multicolor FCCS and FRET Imaging. J. Am. Chem. Soc. 134, 7913-7923 (2012).
26. Pletnev, S. et al. Orange Fluorescent Proteins: Structural Studies of LSSmOrange, PSmOrange and PSmOrange2. PLoS ONE 9, e99136 (2014).
27. Ast, C., Frommer, W. B., Grossmann, G. & De Michele, R. Quantification of Extracellular Ammonium Concentrations and Transporter Activity in Yeast Using AmTrac Fluorescent Sensors. Bio-Protoc. 5, e1372 (2015).
28. Marini, A. M., Soussi-Boudekou, S., Vissers, S. & Andre, B. A family of ammonium transporters in Saccharomyces cerevisiae. Mol. Cell. Biol. 17, 4282-4293 (1997).
29. Schiestl, R. H. & Gietz, R. D. High efficiency transformation of intact yeast cells using single stranded nucleic acids as a carrier. Curr. Genet. 16, 339-346 (1989).
30. Bers, D. M., Patton, C. W. & Nuccitelli, R. A practical guide to the preparation of Ca(2+) buffers. Methods Cell Biol. 99, 1-26 (2010).

Although the presently disclosed technology has been described with reference to the above examples, it will be understood that modifications and variations are encompassed within the spirit and scope of the disclosure and application of the disclosed technology.

References referred to and cited herein are incorporated in their entirety herein by reference.

SEQUENCE LISTING

mCerulean (SEQ ID NO: 2)
MVSKGEELFTGVVPILVELDGDVNGHKFSVSGEGEGDATYGKLTLKFICTTGKLPVPWPTLVTTLTWGVQCFARYPDHM
KQHDFFKSAMPEGYVQERTIFFKDDGNYKTRAEVKFEGDTLVNRIELKGIDFKEDGNILGHKLEYNAISDNVYITADKQ
KNGIKANFKIRHNIEDGSVQLADHYQQNTPIGDGPVLLPDNHYLSTQSKLSKDPNEKRDHMVLLEFVTAAGITLGMDEL
YK
GFP (SEQ ID NO: 3)
MSKGEELFTGVVPVLVELDGDVNGQKFSVSGEGEGDATYGKLTLNFICTTGKLPVPWPTLVTTFSYGVQCFSRYPDHMK
QHDFFKSAMPEGYVQERTIFYKDDGNYKTRAEVKFEGDTLVNRIELKGIDFKEDGNILGHKMEYNYNSHNVYIMGDKPK
NGIKVNFKIRHNIKDGSVQLADHYQQNTPIGDGPVLLPDNHYLSTQSALSKDPNEKRDHMILLEFVTAARITHGMDELY
K
EGFP (SEQ ID NO: 4)
MVSKGEELFTGVVPILVELDGDVNGHKFSVSGEGEGDATYGKLTLKFICTTGKLPVPWPTLVTTLTYGVQCFSRYPDHM
KQHDFFKSAMPEGYVQERTIFFKDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEKRDHMVLLEFVTAAGITLGMDEL
YK
GFP (SEQ ID NO: 5)
MSKGEELFTGVVPILVELDGDVNGQKFSVSGEGEGDATYGKLTLKFICTTGKLPVPWPTLVTTFSYGVQCFSRYPDHMK
QHDFFKSAMPEGYVQERTIFYKDDGNYKTRAEVKFEGDTLVNRIELKGIDFKEDGNILGHKMEYNYNSHNVYIMADKPK
NGIKVNFKIRHNIKDGSVQLADHYQQNTPIGDGPVLLPDNHYLSTQSALSKDPNEKRDHMILLEFVTAAGITHGMDELY
K
cpsfGFP (SEQ ID NO: 6)
AACAGCCATAACGTGTATATTACCGCGGATAAACAGAAAAACGGCATTAAAGCGAACTTTACCGTGCGCCATAACGTGG
AAGATGGCAGCGTGCAGCTGGCGGATCATTATCAGCAGAACACCCCGATTGGCGATGGCCCGGTGCTGCTGCCGGATAA
CCATTATCTGAGCACCCAGACCAAGCTGAGCAAAGATCCGAACGAAAAACGCGATCACATGGTGCTGCTGGAATTTGTG
ACCGCAGCGGGCATTACACACGGCATGGATGAACTGTATGGCGGCACCGGCGGCAGCGCGAGCCAGGGCGAAGAACTGT
TTACCGGCGTGGTGCCGATTCTGGTGGAACTGGATGGCGATGTGAACGGCCATAAATTTAGCGTGCGCGGCGAAGGCGA
AGGCGATGCGACCATTGGCAAACTGACCCTGAAATTTATTTCCACCACCGGCAAACTACCGGTGCCGTGGCCGACCCTG
GTGACCACCTTAACCTATGGCGTGCAGTGCTTTAGCCGCTATCCGGATCATATGAAACGCCATGATTTTTTTAAAAGCG
CGATGCCGGAAGGCTATGTGCAGGAACGCACCATTAGCTTTAAAGATGATGGCAAATATAAAACCCGCGCGGTGGTGAA
ATTTGAAGGCGATACCCTGGTGAACCGCATTGAACTGAAAGGCACCGATTTTAAAGAAGATGGCAACATTCTGGGGCAT
AAACTGGAATATAACTTT
cpsfGFP (SEQ ID NO: 7)
NSHNVYITADKQKNGIKANFTVRHNVEDGSVQLADHYQQNTPIGDGPVLLPDNHYLSTQTKLSKDPNEKRDHMVLLEFV
TAAGITHGMDELYGGTGGSASQGEELFTGVVPILVELDGDVNGHKFSVRGEGEGDATIGKLTLKFISTTGKLPVPWPTL
VTTLTYGVQCFSRYPDHMKRHDFFKSAMPEGYVQERTISFKDDGKYKTRAVVKFEGDTLVNRIELKGTDFKEDGNILGH
KLEYNF
cpsfGFP-T78H (SEQ ID NO: 9)
AACAGCCATAACGTGTATATTACCGCGGATAAACAGAAAAACGGCATTAAAGCGAACTTTcaCGTGCGCCATAACGTGG
AAGATGGCAGCGTGCAGCTGGCGGATCATTATCAGCAGAACACCCCGATTGGCGATGGCCCGGTGCTGCTGCCGGATAA
CCATTATCTGAGCACCCAGACCAAGCTGAGCAAAGATCCGAACGAAAAACGCGATCACATGGTGCTGCTGGAATTTGTG
ACCGCAGCGGGCATTACACACGGCATGGATGAACTGTATGGCGGCACCGGCGGCAGCGCGAGCCAGGGCGAAGAACTGT
TTACCGGCGTGGTGCCGATTCTGGTGGAACTGGATGGCGATGTGAACGGCCATAAATTTAGCGTGCGCGGCGAAGGCGA
AGGCGATGCGACCATTGGCAAACTGACCCTGAAATTTATTTCCACCACCGGCAAACTACCGGTGCCGTGGCCGACCCTG
GTGACCACCTTAACCTATGGCGTGCAGTGCTTTAGCCGCTATCCGGATCATATGAAACGCCATGATTTTTTTAAAAGCG
CGATGCCGGAAGGCTATGTGCAGGAACGCACCATTAGCTTTAAAGATGATGGCAAATATAAAACCCGCGCGGTGGTGAA
ATTTGAAGGCGATACCCTGGTGAACCGCATTGAACTGAAAGGCACCGATTTTAAAGAAGATGGCAACATTCTGGGGCAT
AAACTGGAATATAACTTT
cpsfGFP-T78H (SEQ ID NO: 10)
NSHNVYITADKQKNGIKANFHVRHNVEDGSVQLADHYQQNTPIGDGPVLLPDNHYLSTQTKLSKDPNEKRDHMVLLEFV
TAAGITHGMDELYGGTGGSASQGEELFTGVVPILVELDGDVNGHKFSVRGEGEGDATIGKLTLKFISTTGKLPVPWPTL
VTTLTYGVQCFSRYPDHMKRHDFFKSAMPEGYVQERTISFKDDGKYKTRAVVKFEGDTLVNRIELKGTDFKEDGNILGH
KLEYNF
mVenus (SEQ ID NO: 11)
ATGGTGAGCAAGGGCGAGGAGCTGTTCACCGGGGTGGTGCCCATCCTGGTCGAGCTGGACGGCGACGTAAACGGCCACA
AGTTCAGCGTGTCCGGCGAGGGCGAGGGCGATGCCACCTACGGCAAGCTGACCCTGAAGCTGATCTGCACCACCGGCAA
GCTGCCCGTGCCCTGGCCCACCCTCGTGACCACCCTGGGCTACGGCCTGCAGTGCTTCGCCCGCTACCCCGACCACATG
AAGCAGCACGACTTCTTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAAGGGCATCGACTTCAA
GGAGGACGGCAACATCCTGGGGCACAAGCTGGAGTACAACTACAACAGCCACAACGTCTATATCACCGCCGACAAGCAG
AAGAACGGCATCAAGGCCAACTTCAAGATCCGCCACAACATCGAGGACGGCGGCGTGCAGCTCGCCGACCACTACCAGC
AGAACACCCCCATCGGCGACGGCCCCGTGCTGCTGCCCGACAACCACTACCTGAGCTACCAGTCCAAGCTGAGCAAAGA
CCCCAACGAGAAGCGCGATCACATGGTCCTGCTGGAGTTCGTGACCGCCGCCGGGATCACTCTCGGCATGGACGAGCTG
TACAAGTAA
mVenus (SEQ ID NO: 12)
MVSKGEELFTGVVPILVELDGDVNGHKFSVSGEGEGDATYGKLTLKLICTTGKLPVPWPTLVTTLGYGLQCFARYPDHM
KQHDFFKSAMPEGYVQERTIFFKDDGNYKTRAEVKFEGDTLVNRIELKGIDFKEDGNILGHKLEYNYNSHNVYITADKQ
KNGIKANFKIRHNIEDGGVQLADHYQQNTPIGDGPVLLPDNHYLSYQSKLSKDPNEKRDHMVLLEFVTAAGITLGMDEL
YK
mCherry (SEQ ID NO: 13)
ATGGTGAGCAAGGGCGAGGAGGATAACATGGCCATCATCAAGGAGTTCATGCGCTTCAAGGTGCACATGGAGGGCTCCG
TGAACGGCCACGAGTTCGAGATCGAGGGCGAGGGCGAGGGCCGCCCCTACGAGGGCACCCAGACCGCCAAGCTGAAGGT
GACCAAGGGTGGCCCCCTGCCCTTCGCCTGGGACATCCTGTCCCCTCAGTTCATGTACGGCTCCAAGGCCTACGTGAAG
CACCCCGCCGACATCCCCGACTACTTGAAGCTGTCCTTCCCCGAGGGCTTCAAGTGGGAGCGCGTGATGAACTTCGAGG
ACGGCGGCGTGGTGACCGTGACCCAGGACTCCTCCCTGCAGGACGGCGAGTTCATCTACAAGGTGAAGCTGCGCGGCAC
CAACTTCCCCTCCGACGGCCCCGTAATGCAGAAGAAGACCATGGGCTGGGAGGCCTCCTCCGAGCGGATGTACCCCGAG
GACGGCGCCCTGAAGGGCGAGATCAAGCAGAGGCTGAAGCTGAAGGACGGCGGCCACTACGACGCTGAGGTCAAGACCA
CCTACAAGGCCAAGAAGCCCGTGCAGCTGCCCGGCGCCTACAACGTCAACATCAAGTTGGACATCACCTCCCACAACGA
GGACTACACCATCGTGGAACAGTACGAACGCGCCGAGGGCCGCCACTCCACCGGCGGCATGGACGAGCTGTACAAGTAG
mCherry (SEQ ID NO: 14)
MVSKGEEDNMAIIKEFMRFKVHMEGSVNGHEFEIEGEGEGRPYEGTQTAKLKVTKGGPLPFAWDILSPQFMYGSKAYVK
HPADIPDYLKLSFPEGFKWERVMNFEDGGVVTVTQDSSLQDGEFIYKVKLRGTNFPSDGPVMQKKTMGWEASSERMYPE
DGALKGEIKQRLKLKDGGHYDAEVKTTYKAKKPVQLPGAYNVNIKLDITSHNEDYTIVEQYERAEGRHSTGGMDELYK
mKate (SEQ ID NO: 16)
MVSKGEELIKENMHMKLYMEGTVNNHHFKCTSEGEGKPYEGTQTMRIKVVEGGPLPFAFDILATSFMYGSKTFINHTQG
IPDFWKQSFPEGFTWERVTTYEDGGVLTATQDTSLQDGCLIYNVKIRGVNFPSNGPVMQKKTLGWEANTEMLYPADGGL
EGRGDMALKLVGGGHLICNLKTTYRSKKPAKNLKMPGVYYVDRRLERIKEADKETYVEQHEVAVARYCDLPSKLGHKLN
mApple (SEQ ID NO: 17)
ATGGTGAGCAAGGGCGAGGAGAATAACATGGCCATCATCAAGGAGTTCATGCGCTTCAAGGTGCACATGGAGGGCTCCG
TGAACGGCCACGAGTTCGAGATCGAGGGCGAGGGCGAGGGCCGCCCCTACGAGGCCTTTCAGACCGCTAAGCTGAAGGT
GACCAAGGGTGGCCCCCTGCCCTTCGCCTGGGACATCCTGTCCCCTCAGTTCATGTACGGCTCCAAGGTCTACATTAAG
CACCCAGCCGACATCCCCGACTACTTCAAGCTGTCCTTCCCCGAGGGCTTCAGGTGGGAGCGCGTGATGAACTTCGAGG
ACGGCGGCATTATTCACGTTAACCAGGACTCCTCCCTGCAGGACGGCGTGTTCATCTACAAGGTGAAGCTGCGCGGCAC
CAACTTCCCCTCCGACGGCCCCGTAATGCAGAAGAAGACCATGGGCTGGGAGGCCTCCGAGGAGCGGATGTACCCCGAG
GACGGCGCCCTGAAGAGCGAGATCAAGAAGAGGCTGAAGCTGAAGGACGGCGGCCACTACGCCGCCGAGGTCAAGACCA
CCTACAAGGCCAAGAAGCCCGTGCAGCTGCCCGGCGCCTACATCGTCGACATCAAGTTGGACATCGTGTCCCACAACGA
GGACTACACCATCGTGGAACAGTACGAACGCGCCGAGGGCCGCCACTCCACCGGCGGCATGGACGAGCTGTACAAGTAA
mApple (SEQ ID NO: 18)
MVSKGEENNMAIIKEFMRFKVHMEGSVNGHEFEIEGEGEGRPYEAFQTAKLKVTKGGPLPFAWDILSPQFMYGSKVYIK
HPADIPDYFKLSFPEGFRWERVMNFEDGGIIHVNQDSSLQDGVFIYKVKLRGTNFPSDGPVMQKKTMGWEASEERMYPE
DGALKSEIKKRLKLKDGGHYAAEVKTTYKAKKPVQLPGAYIVDIKLDIVSHNEDYTIVEQYERAEGRHSTGGMDELYK
LSSmOrange (SEQ ID NO: 19)
ATGGTGAGCAAGGGCGAGGAGAATAACATGGCCATCATCAAGGAGTTCATGCGCTTCAAGGTGCGCATGGAGGGCTCCG
TGAACGGCCACGAGTTCGAGATCGAGGGCGAGGGCGAGGGCCGCCCCTACGAGGGCTTTCAGACCGTTAAGCTGAAGGT
GACCAAGGGTGGCCCCCTGCCCTTCGCCTGGGACATCTTGTCCCCTCAGTTCACCTACGGCTCCAAGGCCTACGTGAAG
CACCCCGCCGACATCCCCGACTACCTCAAGCTGTCCTTCCCCGAGGGCTTCAAGTGGGAGCGCGTGATGAACTTCGAGG
ACGGCGGCGTGGTGACCGTGACTCAGGACTCCTCCCTGCAGGACGGCGAGTTCATCTACAAGGTGAAGCTGCGCGGCAC
CAACTTCCCCTCCGACGGCCCCGTAATGCAGAAGAAGACCATGGGCATGGAGGCCTCCTCCGAGCGGATGTACCCCGAG
GACGGCGCCCTGAAGGGCGAGGACAAGCTCAGGCTGAAGCTGAAGGACGGCGGCCACTACACCTCCGAGGTCAAGACCA
CCTACAAGGCCAAGAAGCCCGTGCAGTTGCCCGGCGCCTACATCGTCGACATCAAGTTGGACATCACCTCCCACAACGA
GGACTACACCATCGTGGAACAGTACGAACGCGCCGAGGGCCGCCACTCCACCGGCGGCATGGACGAGCTGTACAAGTAA
LSSmOrange (SEQ ID NO: 20)
MVSKGEENNMAIIKEFMRFKVRMEGSVNGHEFEIEGEGEGRPYEGFQTVKLKVTKGGPLPFAWDILSPQFTYGSKAYVK
HPADIPDYLKLSFPEGFKWERVMNFEDGGVVTVTQDSSLQDGEFIYKVKLRGTNFPSDGPVMQKKTMGMEASSERMYPE
DGALKGEDKLRLKLKDGGHYTSEVKTTYKAKKPVQLPGAYIVDIKLDITSHNEDYTIVEQYERAEGRHSTGGMDELYK
mHoneydew (SEQ ID No: 21)
ATGGTGAGCAAGGGCGAGGAGGTCATCAAGGAGTTCATGCGCTTCAAGGTGCGCATGGAGGGCTCCGTGAACGGCCACG
AGTTCGAGATCGAGGGCGAGGGCGAGGGCCGCCCCTACGAGGGCACCCAGACCGCCAAGCTGAAGGTGACCAAGGGCGG
CCCCCTGCCCTTCGCCTGGGACATCCTGTCCCCTCAGTTCATGTGGGGCTCCAAGGCCTACGTGAAGCACCCCGCCGAC
ATCCCCGACTACTTGAAGCTGTCCTTCCCCGAGGGCTTCAAGTGGGAGCGCGTGATGAACTTCGAGGACGGCGGCGTGG
TGACCGTGACCCAGGACTCCTCCCTGCAGGACGGCGAGTTCATCTACAAGGTGAAGCTGCGCGGCACCAACTTCCCCTC
CGACGGCCCCGTAATGCAGAAGAAGACCATGGGCTGGGCGGCCACCACCGAGCGGATGTACCCCGAGGACGGCGCCCTG
AAGGGCGAGATCAAGATGAGGCTGAAGCTGAAGGACGGCGGCCACTACGACGCCGAGGTCAAGACCACCTACATGGCCA
AGAAGCCCGTGCAGCTGCCCGGCGCCTACAAGATTGACGGGAAGCTGGACATCACCTCCCACAACGAGGACTACACCAT
CGTGGAACAGTACGAGCGCGCCGAGGGCGGCCACTCCACCGGCGGCATGGACGAGCTGTACAAG
mHoneydew (SEQ ID No: 22)
MVSKGEEVIKEFMRFKVRMEGSVNGHEFEIEGEGEGRPYEGTQTAKLKVTKGGPLPFAWDILSPQFMWGSKAYVKHPAD
IPDYLKLSFPEGFKWERVMNFEDGGVVTVTQDSSLQDGEFIYKVKLRGTNFPSDGPVMQKKTMGWAATTERMYPEDGAL
KGEIKMRLKLKDGGHYDAEVKTTYMAKKPVQLPGAYKIDGKLDITSHNEDYTIVEQYERAEGGHSTGGMDELYK
mBanana (SEQ ID NO: 23)
ATGGTGAGCAAGGGCGAGGAGAATAACATGGCCGTCATCAAGGAGTTCATGCGCTTCAAGGTGCGCATGGAGGGCTCCG
TGAACGGCCACGAGTTCGAGATCGAGGGCGAGGGCGAGGGCCGCCCCTACGAGGGCACCCAGACCGCCAAGCTGAAGGT
GACCAAGGGTGGCCCCCTGCCCTTCGCCTGGGACATCCTGTCCCCTCAGTTCTGTTACGGCTCCAAGGCCTACGTGAAG
CACCCCACTGGTATCCCCGACTACTTCAAGCTGTCCTTCCCCGAGGGCTTCAAGTGGGAGCGCGTGATGAACTTCGAGG
ACGGCGGCGTGGTGACCGTGGCTCAGGACTCCTCCCTGCAGGACGGCGAGTTCATCTACAAGGTGAAGCTGCGCGGCAC
CAACTTCCCCTCCGACGGCCCCGTAATGCAGAAGAAGACCATGGGCTGGGAGGCCTCCTCCGAGCGGATGTACCCCGAG
GACGGCGCCCTGAAGGGCGAGATCAAGATGAGGCTGAAGCTGAAGGACGGCGGCCACTACAGCGCCGAGACCAAGACCA
CCTACAAGGCCAAGAAGCCCGTGCAGTTGCCCGGCGCCTACATAGCCGGCGAGAAGATCGACATCACCTCCCACAATGA
GGACTACACTATCGTGGAATTGTACGAGCGCGCCGAGGGCCGCCACTCCACCGGCGGCATGGACGAGCTGTACAAGTAG
mBanana (SEQ ID NO: 24)
MVSKGEENNMAVIKEFMRFKVRMEGSVNGHEFEIEGEGEGRPYEGTQTAKLKVTKGGPLPFAWDILSPQFCYGSKAYVK
HPTGIPDYFKLSFPEGFKWERVMNFEDGGVVTVAQDSSLQDGEFIYKVKLRGTNFPSDGPVMQKKTMGWEASSERMYPE
DGALKGEIKMRLKLKDGGHYSAETKTTYKAKKPVQLPGAYIAGEKIDITSHNEDYTIVELYERAEGRHSTGGMDELYK
cpEGFP (SEQ ID NO: 25)
AACGTCTATATCAaGGCCGACAAGCAGAAGAACGGCATCAAGGcGAACTTCAAGATCCGCCACAACATCGAGGACGGCg
GCGTGCAGCTCGCCtACCACTACCAGCAGAACACCCCCATCGGCGACGGCCCCGTGCTGCTGCCCGACAACCACTACCT
GAGCgtCCAGTCCaagCTGAGCAAAGACCCCAACGAGAAGCGCGATCACATGGTCCTGCTGGAGTTCGTGACCGCCGCC
GGGATCACTCTCGGCATGGACGAGCTGTACAAGGGTGGTACCGGTGGATCTATGGTGAGCAAGGGCGAGGAGCTGTTCA
CCGGGGTGGTGCCCATCCTGGTCGAGCTGGACGGCGACGTAAACGGCCACAAGTTCAGCGTGTCCGGCGAGGGCGAGGG
CGATGCCACCTACGGCAAGCTGACCCTGAAGTTCATCTGCACCACCGGCAAGCTGCCCGTGCCCTGGCCCACCCTCGTG
ACCACCCTGACCTACGGCGTGCAGTGCTTCAGCCGCTACCCCGACCACATGAAGCAGCACGACTTCTTCAAGTCCGCCA
TGCCCGAAGGCTACaTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAACATCCTGGGGCACAAG
CTGGAGTACAAC
cpEGFP (SEQ ID NO: 26)
NVYIKADKQKNGIKANFKIRHNIEDGGVQLAYHYQQNTPIGDGPVLLPDNHYLSVQSKLSKDPNEKRDHMVLLEFVTAA
GITLGMDELYKGGTGGSMVSKGEELFTGVVPILVELDGDVNGHKFSVSGEGEGDATYGKLTLKFICTTGKLPVPWPTLV
TTLTYGVQCFSRYPDHMKQHDFFKSAMPEGYIQERTIFFKDDGNYKTRAEVKFEGDTLVNRIELKGIDFKEDGNILGHK
LEYN
GO-Matroshka-LS-FN (SEQ ID NO: 29)
ttgtccAACGTGTATATTACCGCGGATAAACAGAAAAACGGCATTAAAGCGAACTTTACCGTGCGCCATAACGTGGAAG
ATGGCAGCGTGCAGCTGGCGGATCATTATCAGCAGAACACCCCGATTGGCGATGGCCCGGTGCTGCTGCCGGATAACCA
TTATCTGAGCACCCAGACCAAGCTGAGCAAAGATCCGAACGAAAAACGCGATCACATGGTGCTGCTGGAATTTGTGACC
GCAGCGGGCATTACACACGGCATGGATGAACTGTATGGCGGCACCatggtgagcaagggcgaggagaataacatggcca
tcatcaaggagttcatgcgcttcaaggtgcgcatggagggctccgtgaacggccacgagttcgagatcgagggcgaggg
cgagggccgcccctacgagggctttcagaccgttaagctgaaggtgaccaagggtggccccctgcccttcgcctgggac
atcttgtcccctcagttcacctacggctccaaggcctacgtgaagcaccccgccgacatccccgactacctcaagctgt
ccttccccgagggcttcaagtgggagcgcgtgatgaacttcgaggacggcggcgtggtgaccgtgactcaggactcctc
cctgcaggacggcgagttcatctacaaggtgaagctgcgcggcaccaacttcccctccgacggccccgtaatgcagaag
aagaccatgggcatggaggcctcctccgagcggatgtaccccgaggacggcgccctgaagggcgaggacaagctcaggc
tgaagctgaaggacggcggccactacacctccgaggtcaagaccacctacaaggccaagaagcccgtgcagttgcccgg
cgcctacatcgtcgacatcaagttggacatcacctcccacaacgaggactacaccatcgtggaacagtacgaacgcgcc
gagggccgccactccaccggcggcatggacgagctgtacaagGGCGGCAGCGCGAGCCAGGGCGAAGAACTGTTTACCG
GCGTGGTGCCGATTCTGGTGGAACTGGATGGCGATGTGAACGGCCATAAATTTAGCGTGCGCGGCGAAGGCGAAGGCGA
TGCGACCATTGGCAAACTGACCCTGAAATTTATTTCCACCACCGGCAAACTACCGGTGCCGTGGCCGACCCTGGTGACC
ACCTTAACCTATGGCGTGCAGTGCTTTAGCCGCTATCCGGATCATATGAAACGCCATGATTTTTTTAAAAGCGCGATGC
CGGAAGGCTATGTGCAGGAACGCACCATTAGCTTTAAAGATGATGGCAAATATAAAACCCGCGCGGTGGTGAAATTTGA
AGGCGATACCCTGGTGAACCGCATTGAACTGAAAGGCACCGATTTTAAAGAAGATGGCAACATTCTGGGGCATAAACTG
GAATATAACtttaat
GO-Matroshka-LS-FN (SEQ ID NO: 30)
LSNVYITADKQKNGIKANFTVRHNVEDGSVQLADHYQQNTPIGDGPVLLPDNHYLSTQTKLSKDPNEKRDHMVLLEFVT
AAGITHGMDELYGGTMVSKGEENNMAIIKEFMRFKVRMEGSVNGHEFEIEGEGEGRPYEGFQTVKLKVTKGGPLPFAWD
ILSPQFTYGSKAYVKHPADIPDYLKLSFPEGFKWERVMNFEDGGVVTVTQDSSLQDGEFIYKVKLRGTNFPSDGPVMQK
KTMGMEASSERMYPEDGALKGEDKLRLKLKDGGHYTSEVKTTYKAKKPVQLPGAYIVDIKLDITSHNEDYTIVEQYERA
EGRHSTGGMDELYKGGSASQGEELFTGVVPILVELDGDVNGHKFSVRGEGEGDATIGKLTLKFISTTGKLPVPWPTLVT
TLTYGVQCFSRYPDHMKRHDFFKSAMPEGYVQERTISFKDDGKYKTRAVVKFEGDTLVNRIELKGTDFKEDGNILGHKL
EYNFN
AtAMT1: 3 1-498 protein (SEQ ID NO: 34)
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKGGRA
IALRGHSASLVVLGTFLLWFGWYGFNPGSFTKILVPYNSGSNYGQWSGIGRTAVNTTLSGCTAALTTLFGKRLLSGHWN
VTDVCNGLLGGFAAITAGCSVVEPWAAIVCGFMASVVLIGCNKLAELVQYDDPLEAAQLHGGCGAWGLIFVGLFAKEKY
LNEVYGATPGRPYGLFMGGGGKLLGAQLVQILVIVGWVSATMGTLFFILKRLNLLRISEQHEMQGMDMTRHGGFAYIYH
DNDDESHRVDPGSPFPRSATPPRV
dTomato (SEQ ID NO: 83)
ATGGTGAGCAAGGGCGAGGAGGTCATCAAAGAGTTCATGCGCTTCAAGGTGCGCATGGAGGGCTCCATGAACGGCCACG
AGTTCGAGATCGAGGGCGAGGGCGAGGGCCGCCCCTACGAGGGCACCCAGACCGCCAAGCTGAAGGTGACCAAGGGCGG
CCCCCTGCCCTTCGCCTGGGACATCCTGTCCCCCCAGTTCATGTACGGCTCCAAGGCGTACGTGAAGCACCCCGCCGAC
ATCCCCGATTACAAGAAGCTGTCCTTCCCCGAGGGCTTCAAGTGGGAGCGCGTGATGAACTTCGAGGACGGCGGTCTGG
TGACCGTGACCCAGGACTCCTCCCTGCAGGACGGCACGCTGATCTACAAGGTGAAGATGCGCGGCACCAACTTCCCCCC
CGACGGCCCCGTAATGCAGAAGAAGACCATGGGCTGGGAGGCCTCCACCGAGCGCCTGTACCCCCGCGACGGCGTGCTG
AAGGGCGAGATCCACCAGGCCCTGAAGCTGAAGGACGGCGGCCACTACCTGGTGGAGTTCAAGACCATCTACATGGCCA
AGAAGCCCGTGCAACTGCCCGGCTACTACTACGTGGACACCAAGCTGGACATCACCTCCCACAACGAGGACTACACCAT
CGTGGAACAGTACGAGCGCTCCGAGGGCCGCCACCACCTGTTCCTGTACGGCATGGACGAGCTGTACAAG
dTomato (SEQ ID NO: 84)
MVSKGEEVIKEFMRFKVRMEGSMNGHEFEIEGEGEGRPYEGTQTAKLKVTKGGPLPFAWDILSPQFMYGSKAYVKHPAD
IPDYKKLSFPEGFKWERVMNFEDGGLVTVTQDSSLQDGTLIYKVKMRGTNFPPDGPVMQKKTMGWEASTERLYPRDGVL
KGEIHQALKLKDGGHYLVEFKTIYMAKKPVQLPGYYYVDTKLDITSHNEDYTIVEQYERSEGRHHLFLYGMDELYK
tdTomato (SEQ ID NO: 85)
ATGGTGAGCAAGGGCGAGGAGGTCATCAAAGAGTTCATGCGCTTCAAGGTGCGCATGGAGGGCTCCATGAACGGCCACG
AGTTCGAGATCGAGGGCGAGGGCGAGGGCCGCCCCTACGAGGGCACCCAGACCGCCAAGCTGAAGGTGACCAAGGGCGG
CCCCCTGCCCTTCGCCTGGGACATCCTGTCCCCCCAGTTCATGTACGGCTCCAAGGCGTACGTGAAGCACCCCGCCGAC
ATCCCCGATTACAAGAAGCTGTCCTTCCCCGAGGGCTTCAAGTGGGAGCGCGTGATGAACTTCGAGGACGGCGGTCTGG
TGACCGTGACCCAGGACTCCTCCCTGCAGGACGGCACGCTGATCTACAAGGTGAAGATGCGCGGCACCAACTTCCCCCC
CGACGGCCCCGTAATGCAGAAGAAGACCATGGGCTGGGAGGCCTCCACCGAGCGCCTGTACCCCCGCGACGGCGTGCTG
AAGGGCGAGATCCACCAGGCCCTGAAGCTGAAGGACGGCGGCCACTACCTGGTGGAGTTCAAGACCATCTACATGGCCA
AGAAGCCCGTGCAACTGCCCGGCTACTACTACGTGGACACCAAGCTGGACATCACCTCCCACAACGAGGACTACACCAT
CGTGGAACAGTACGAGCGCTCCGAGGGCCGCCACCACCTGTTCCTGGGGCATGGCACCGGCAGCACCGGCAGCGGCAGC
TCCGGCACCGCCTCCTCCGAGGACAACAACATGGCCGTCATCAAAGAGTTCATGCGCTTCAAGGTGCGCATGGAGGGCT
CCATGAACGGCCACGAGTTCGAGATCGAGGGCGAGGGCGAGGGCCGCCCCTACGAGGGCACCCAGACCGCCAAGCTGAA
GGTGACCAAGGGCGGCCCCCTGCCCTTCGCCTGGGACATCCTGTCCCCCCAGTTCATGTACGGCTCCAAGGCGTACGTG
AAGCACCCCGCCGACATCCCCGATTACAAGAAGCTGTCCTTCCCCGAGGGCTTCAAGTGGGAGCGCGTGATGAACTTCG
AGGACGGCGGTCTGGTGACCGTGACCCAGGACTCCTCCCTGCAGGACGGCACGCTGATCTACAAGGTGAAGATGCGCGG
CACCAACTTCCCCCCCGACGGCCCCGTAATGCAGAAGAAGACCATGGGCTGGGAGGCCTCCACCGAGCGCCTGTACCCC
CGCGACGGCGTGCTGAAGGGCGAGATCCACCAGGCCCTGAAGCTGAAGGACGGCGGCCACTACCTGGTGGAGTTCAAGA
CCATCTACATGGCCAAGAAGCCCGTGCAACTGCCCGGCTACTACTACGTGGACACCAAGCTGGACATCACCTCCCACAA
CGAGGACTACACCATCGTGGAACAGTACGAGCGCTCCGAGGGCCGCCACCACCTGTTCCTGTACGGCATGGACGAGCTG
TACAAGTAG
tdTomato (SEQ ID NO: 86)
MVSKGEEVIKEFMRFKVRMEGSMNGHEFEIEGEGEGRPYEGTQTAKLKVTKGGPLPFAWDILSPQFMYGSKAYVKHPAD
IPDYKKLSFPEGFKWERVMNFEDGGLVTVTQDSSLQDGTLIYKVKMRGTNFPPDGPVMQKKTMGWEASTERLYPRDGVL
KGEIHQALKLKDGGHYLVEFKTIYMAKKPVQLPGYYYVDTKLDITSHNEDYTIVEQYERSEGRHHLFLGHGTGSTGSGS
SGTASSEDNNMAVIKEFMRFKVRMEGSMNGHEFEIEGEGEGRPYEGTQTAKLKVTKGGPLPFAWDILSPQFMYGSKAYV
KHPADIPDYKKLSFPEGFKWERVMNFEDGGLVTVTQDSSLQDGTLIYKVKMRGTNFPPDGPVMQKKTMGWEASTERLYP
RDGVLKGEIHQALKLKDGGHYLVEFKTIYMAKKPVQLPGYYYVDTKLDITSHNEDYTIVEQYERSEGRHHLFLYGMDEL
YK
mTangerine (SEQ ID NO: 87)
ATGGTGAGCAAGGGCGAGGAGGTCATCAAGGAGTTCATGCGCTTCAAGGTGCGCATGGAGGGCTCCGTGAACGGCCACG
AGTTCGAGATCGAGGGCGAGGGCGAGGGCCGCCCCTACGAGGGCACCCAGACCGCCAAGCTGAAGGTGACCAAGGGCGG
CCCCCTGCCCTTCGCCTGGGACATCCTGTCCCCTCAGTTCTGTTACGGCTCCAAGGCCTACGTGAAGCACCCCGCCGAC
ATCCCCGACTACTTGAAGCTGTCCTTCCCCGAGGGCTTCAAGTGGGAGCGCGTGATGAACTTCGAGGACGGCGGCGTGG
TGACCGTGACCCAGGACTCCTCCCTGCAGGACGGCGAGTTCATCTACAAGGTGAAGCTGCGCGGCACCAACTTCCCCTC
CGACGGCCCCGTAATGCAGAAGAAGACCATGGGCTGGGAGGCCTCCTCCGAGCGGATGTACCCCGAGGACGGCGCCCTG
AAGGGCGAGATCAAGATGAGGCTGAAGCTGAAGGACGGCGGCCACTACGACGCCGAGGTCAAGACCACCTACATGGCCA
AGAAGCCCGTGCAGCTGCCCGGCGCCTACAAGACCGACATCAAGCTGGACATCACCTCCCACAACGAGGACTACACCAT
CGTGGAATTGTACGAGCGCGCCGAGGGCCGCCACTCCACCGGCGGCATGGACGAGCTGTACAAG
mTangerine (SEQ ID NO: 88)
MVSKGEEVIKEFMRFKVRMEGSVNGHEFEIEGEGEGRPYEGTQTAKLKVTKGGPLPFAWDILSPQFCYGSKAYVKHPAD
IPDYLKLSFPEGFKWERVMNFEDGGVVTVTQDSSLQDGEFIYKVKLRGTNFPSDGPVMQKKTMGWEASSERMYPEDGAL
KGEIKMRLKLKDGGHYDAEVKTTYMAKKPVQLPGAYKTDIKLDITSHNEDYTIVELYERAEGRHSTGGMDELYK
mStrawberry (SEQ ID NO: 89)
ATGGTGAGCAAGGGCGAGGAGAATAACATGGCCATCATCAAGGAGTTCATGCGCTTCAAGGTGCGCATGGAGGGCTCCG
TGAACGGCCACGAGTTCGAGATCGAGGGCGAGGGCGAGGGCCGCCCCTACGAGGGCACCCAGACCGCCAAGCTGAAGGT
GACCAAGGGTGGCCCCCTGCCCTTCGCCTGGGACATCCTAACCCCCAACTTCACCTACGGCTCCAAGGCCTACGTGAAG
CACCCCGCCGACATCCCCGACTACTTGAAGCTGTCCTTCCCCGAGGGCTTCAAGTGGGAGCGCGTGATGAACTTCGAGG
ACGGCGGCGTGGTGACCGTGACCCAGGACTCCTCCCTGCAGGACGGCGAGTTCATCTACAAGGTGAAGCTGCGCGGCAC
CAACTTCCCCTCCGACGGCCCCGTAATGCAGAAGAAGACCATGGGCTGGGAGGCCTCCTCCGAGCGGATGTACCCCGAG
GACGGCGCCCTGAAGGGCGAGATCAAGATGAGGCTGAAGCTGAAGGACGGCGGCCACTACGACGCTGAGGTCAAGACCA
CCTACAAGGCCAAGAAGCCCGTGCAGCTGCCCGGCGCCTACATCGTCGGCATCAAGTTGGACATCACCTCCCACAACGA
GGACTACACCATCGTGGAACTGTACGAACGCGCCGAGGGCCGCCACTCCACCGGCGGCATGGACGAGCTGTACAAGTAA
mStrawberry (SEQ ID NO: 90)
MVSKGEENNMAIIKEFMRFKVRMEGSVNGHEFEIEGEGEGRPYEGTQTAKLKVTKGGPLPFAWDILTPNFTYGSKAYVK
HPADIPDYLKLSFPEGFKWERVMNFEDGGVVTVTQDSSLQDGEFIYKVKLRGTNFPSDGPVMQKKTMGWEASSERMYPE
DGALKGEIKMRLKLKDGGHYDAEVKTTYKAKKPVQLPGAYIVGIKLDITSHNEDYTIVELYERAEGRHSTGGMDELYK
mRuby (SEQ ID NO: 91)
ATGAACAGCCTGATCAAAGAAAACATGCGGATGAAGGTGGTGCTGGAAGGCAGCGTGAACGGCCACCAGTTCAAGTGCA
CCGGCGAGGGCGAGGGCAACCCCTACATGGGCACCCAGACCATGCGGATCAAAGTGATCGAGGGCGGACCTCTGCCCTT
CGCCTTCGACATCCTGGCCACATCCTTCATGTACGGCAGCCGGACCTTCATCAAGTACCCCAAGGGCATCCCCGATTTC
TTCAAGCAGAGCTTCCCCGAGGGCTTCACCTGGGAGAGAGTGACCAGATACGAGGACGGCGGCGTGATCACCGTGATGC
AGGACACCAGCCTGGAAGATGGCTGCCTGGTGTACCATGCCCAGGTCAGGGGCGTGAATTTTCCCAGCAACGGCGCCGT
GATGCAGAAGAAAACCAAGGGCTGGGAGCCCAACACCGAGATGATGTACCCCGCTGACGGCGGACTGAGAGGCTACACC
CACATGGCCCTGAAGGTGGACGGCGGAGGGCACCTGAGCTGCAGCTTCGTGACCACCTACCGATCCAAGAAAACCGTGG
GCAACATCAAGATGCCCGGCATCCACGCCGTGGACCACCGGCTGGAAAGGCTGGAAGAGTCCGACAACGAGATGTTCGT
GGTGCAGCGGGAGCACGCCGTGGCCAAGTTCGCCGGCCTGGGCGGAGGG
mRuby (SEQ ID NO: 92)
MNSLIKENMRMKVVLEGSVNGHQFKCTGEGEGNPYMGTQTMRIKVIEGGPLPFAFDILATSFMYGSRTFIKYPKGIPDF
FKQSFPEGFTWERVTRYEDGGVITVMQDTSLEDGCLVYHAQVRGVNFPSNGAVMQKKTKGWEPNTEMMYPADGGLRGYT
HMALKVDGGGHLSCSFVTTYRSKKTVGNIKMPGIHAVDHRLERLEESDNEMFVVQREHAVAKFAGLGGG
mRuby2 (SEQ ID NO: 93)
ATGGTGTCTAAGGGCGAAGAGCTGATCAAGGAAAATATGCGTATGAAGGTGGTCATGGAAGGTTCGGTCAACGGCCACC
AATTCAAATGCACAGGTGAAGGAGAAGGCAATCCGTACATGGGAACTCAAACCATGAGGATCAAAGTCATCGAGGGAGG
ACCCCTGCCATTTGCCTTTGACATTCTTGCCACGTCGTTCATGTATGGCAGCCGTACTTTTATCAAGTACCCGAAAGGC
ATTCCTGATTTCTTTAAACAGTCCTTTCCTGAGGGTTTTACTTGGGAAAGAGTTACGAGATACGAAGATGGTGGAGTCG
TCACCGTCATGCAGGACACCAGCCTTGAGGATGGCTGTCTCGTTTACCACGTCCAAGTCAGAGGGGTAAACTTTCCCTC
CAATGGTCCCGTGATGCAGAAGAAGACCAAGGGTTGGGAGCCTAATACAGAGATGATGTATCCAGCAGATGGTGGTCTG
AGGGGATACACTCATATGGCACTGAAAGTTGATGGTGGTGGCCATCTGTCTTGCTCTTTCGTAACAACTTACAGGTCAA
AAAAGACCGTCGGGAACATCAAGATGCCCGGTATCCATGCCGTTGATCACCGCCTGGAAAGGTTAGAGGAAAGTGACAA
TGAAATGTTCGTAGTACAACGCGAACACGCAGTTGCCAAGTTCGCCGGGCTTGGTGGTGGGATGGACGAGCTGTACAAG
mRuby2 (SEQ ID NO: 94)
MVSKGEELIKENMRMKVVMEGSVNGHQFKCTGEGEGNPYMGTQTMRIKVIEGGPLPFAFDILATSFMYGSRTFIKYPKG
IPDFFKQSFPEGFTWERVTRYEDGGVVTVMQDTSLEDGCLVYHVQVRGVNFPSNGPVMQKKTKGWEPNTEMMYPADGGL
RGYTHMALKVDGGGHLSCSFVTTYRSKKTVGNIKMPGIHAVDHRLERLEESDNEMFVVQREHAVAKFAGLGGGMDELYK
mKate2 (SEQ ID NO: 95)
ATGGTGAGCGAGCTGATTAAGGAGAACATGCACATGAAGCTGTACATGGAGGGCACCGTGAACAACCACCACTTCAAGT
GCACATCCGAGGGCGAAGGCAAGCCCTACGAGGGCACCCAGACCATGAGAATCAAGGCGGTCGAGGGCGGCCCTCTCCC
CTTCGCCTTCGACATCCTGGCTACCAGCTTCATGTACGGCAGCAAAACCTTCATCAACCACACCCAGGGCATCCCCGAC
TTCTTTAAGCAGTCCTTCCCCGAGGGCTTCACATGGGAGAGAGTCACCACATACGAAGACGGGGGCGTGCTGACCGCTA
CCCAGGACACCAGCCTCCAGGACGGCTGCCTCATCTACAACGTCAAGATCAGAGGGGTGAACTTCCCATCCAACGGCCC
TGTGATGCAGAAGAAAACACTCGGCTGGGAGGCCTCCACCGAGACCCTGTACCCCGCTGACGGCGGCCTGGAAGGCAGA
GCCGACATGGCCCTGAAGCTCGTGGGCGGGGGCCACCTGATCTGCAACTTGAAGACCACATACAGATCCAAGAAACCCG
CTAAGAACCTCAAGATGCCCGGCGTCTACTATGTGGACAGAAGACTGGAAAGAATCAAGGAGGCCGACAAAGAGACCTA
CGTCGAGCAGCACGAGGTGGCTGTGGCCAGATACTGCGACCTCCCTAGCAAACTGGGGCACAGATGA
mKate2 (SEQ ID NO: 96)
MVSELIKENMHMKLYMEGTVNNHHFKCTSEGEGKPYEGTQTMRIKAVEGGPLPFAFDILATSFMYGSKTFINHTQGIPD
FFKQSFPEGFTWERVTTYEDGGVLTATQDTSLQDGCLIYNVKIRGVNFPSNGPVMQKKTLGWEASTETLYPADGGLEGR
ADMALKLVGGGHLICNLKTTYRSKKPAKNLKMPGVYYVDRRLERIKEADKETYVEQHEVAVARYCDLPSKLGHR
mNeptune (SEQ ID No: 97)
ATGGTGTCTAAGGGCGAAGAGCTGATTAAGGAGAACATGCACATGAAGCTGTACATGGAGGGCACCGTGAACAACCACC
ACTTCAAGTGCACATCCGAGGGCGAAGGCAAGCCCTACGAGGGCACCCAGACCGGCAGAATCAAGGTGGTCGAGGGCGG
CCCTCTCCCCTTCGCCTTCGACATCCTGGCTACCTGCTTCATGTACGGCAGCAAGACCTTCATCAACCACACCCAGGGC
ATCCCCGATTTCTTTAAGCAGTCCTTCCCTGAGGGCTTCACATGGGAGAGAGTCACCACATACGAAGACGGGGGCGTGC
TGACCGCTACCCAGGACACCAGCCTCCAGGACGGCTGCCTCATCTACAACGTCAAGATCAGAGGGGTGAACTTCCCATC
CAACGGCCCTGTGATGCAGAAGAAAACACTCGGCTGGGAGGCCAGTACCGAGACGCTGTACCCCGCTGACGGCGGCCTG
GAAGGCAGATGCGACATGGCCCTGAAGCTCGTGGGCGGGGGCCACCTGATCTGCAACCTGAAGACCACATACAGATCCA
AGAAACCCGCTAAGAACCTCAAGATGCCCGGCGTCTACTTTGTGGACCGCAGACTGGAAAGAATCAAGGAGGCCGACAA
TGAGACCTACGTCGAGCAGCACGAGGTGGCTGTGGCCAGATACTGCGACCTCCCTAGCAAACTGGGGCACAAACTTAAT
GGCATGGACGAGCTGTACAAGTAA
mNeptune (SEQ ID No: 98)
MVSKGEELIKENMHMKLYMEGTVNNHHFKCTSEGEGKPYEGTQTGRIKVVEGGPLPFAFDILATCFMYGSKTFINHTQG
IPDFFKQSFPEGFTWERVTTYEDGGVLTATQDTSLQDGCLIYNVKIRGVNFPSNGPVMQKKTLGWEASTETLYPADGGL
EGRCDMALKLVGGGHLICNLKTTYRSKKPAKNLKMPGVYFVDRRLERIKEADNETYVEQHEVAVARYCDLPSKLGHKLN
GMDELYK
TagRFP-T (SEQ ID NO: 99)
ATGGTGTCTAAGGGCGAAGAGCTGATTAAGGAGAACATGCACATGAAGCTGTACATGGAGGGCACCGTGAACAACCACC
ACTTCAAGTGCACATCCGAGGGCGAAGGCAAGCCCTACGAGGGCACCCAGACCATGAGAATCAAGGTGGTCGAGGGCGG
CCCTCTCCCCTTCGCCTTCGACATCCTGGCTACCAGCTTCATGTACGGCAGCAGAACCTTCATCAACCACACCCAGGGC
ATCCCCGACTTCTTTAAGCAGTCCTTCCCTGAGGGCTTCACATGGGAGAGAGTCACCACATACGAAGACGGGGGCGTGC
TGACCGCTACCCAGGACACCAGCCTCCAGGACGGCTGCCTCATCTACAACGTCAAGATCAGAGGGGTGAACTTCCCATC
CAACGGCCCTGTGATGCAGAAGAAAACACTCGGCTGGGAGGCCAACACCGAGATGCTGTACCCCGCTGACGGCGGCCTG
GAAGGCAGAACCGACATGGCCCTGAAGCTCGTGGGCGGGGGCCACCTGATCTGCAACTTCAAGACCACATACAGATCCA
AGAAACCCGCTAAGAACCTCAAGATGCCCGGCGTCTACTATGTGGACCACAGACTGGAAAGAATCAAGGAGGCCGACAA
AGAGACCTACGTCGAGCAGCACGAGGTGGCTGTGGCCAGATACTGCGACCTCCCTAGCAAACTGGGGCACAAACTTAAT
GGCATGGACGAGCTGTACAAG
TagRFP-T (SEQ ID NO: 100)
MVSKGEELIKENMHMKLYMEGTVNNHHFKCTSEGEGKPYEGTQTMRIKVVEGGPLPFAFDILATSFMYGSRTFINHTQG
IPDFFKQSFPEGFTWERVTTYEDGGVLTATQDTSLQDGCLIYNVKIRGVNFPSNGPVMQKKTLGWEANTEMLYPADGGL
EGRTDMALKLVGGGHLICNFKTTYRSKKPAKNLKMPGVYYVDHRLERIKEADKETYVEQHEVAVARYCDLPSKLGHKLN
GMDELYK
LSS-mKate2 (SEQ ID NO: 101)
ATGAGCGAGCTGATTAAGGAGAACATGCACATGAAGCTGTACATGGAAGGCACCGTGAACAACCACCACTTCAAGTGCA
CATCCGAGGGCGAAGGCAAGCCCTACGAGGGCACCCAGACCATGAGAATCAAGGTGGTCGAGGGCGGCCCTCTACCCTT
CGCCTTCGACATCTTGGCTACCAGCTTCATGTACGGCAGCTACACCTTCATCAACCACACCCAGGGCATCCCCGACTTC
TTTAAGCAGTCCTTCCCTGAGGGCTTCACATGGGAGAGAGTCACCACATACGAAGACGGGGGCGTGCTGACCGCTACCC
AGGACACCAGCCTCCAGGACGGTTGCCTCATCTACAACGTCAAGATCAGAGGGGTGAACTTCACATCCAACGGCCCTGT
GATGCAGAAGAAAACACTCGGCTGGGAGGCCGGCACCGAGATGCTGTACCCCGCTGACGGCGGCCTGGAAGGCAGATCT
GACGACGCCCTGAAGCTCGTGGGCGGGGGCCACCTGATCTGCAACTTGAAGAGCACATACAGATCCAAGAAACCCGCTA
AGAATCTCAAGGTGCCCGGCGTCTACTATGTGGACCGAAGACTGGAAAGAATCAAGGAGGCCGACAAAGAGACCTACGT
CGAGCAGCACGAGGTGGCTGTGGCCAGATACTGCGACCTCCCTAGCAAACTGGGGCACAAGCTTAATTAA
LSS-mKate2 (SEQ ID NO: 102)
MSELIKENMHMKLYMEGTVNNHHFKCTSEGEGKPYEGTQTMRIKVVEGGPLPFAFDILATSFMYGSYTFINHTQGIPDF
FKQSFPEGFTWERVTTYEDGGVLTATQDTSLQDGCLIYNVKIRGVNFTSNGPVMQKKTLGWEAGTEMLYPADGGLEGRS
DDALKLVGGGHLICNLKSTYRSKKPAKNLKVPGVYYVDRRLERIKEADKETYVEQHEVAVARYCDLPSKLGHKLN
mKeima (SEQ ID NO: 103)
ATGGTGAGTGTGATCGCTAAACAAATGACCTACAAGGTTTATATGTCAGGCACGGTCAATGGACACTACTTTGAGGTCG
AAGGCGATGGAAAAGGAAAGCCTTACGAGGGAGAGCAGACAGTAAAGCTCACTGTCACCAAGGGTGGACCTCTGCCATT
TGCTTGGGATATTTTATCACCACAGCTTCAGTACGGAAGCATACCATTCACCAAGTACCCTGAAGACATCCCTGATTAT
TTCAAGCAGTCATTCCCTGAGGGATATACATGGGAGAGGAGCATGAACTTTGAAGATGGTGCAGTGTGTACTGTCAGCA
ATGATTCCAGCATCCAAGGCAACTGTTTCATCTACAATGTCAAAATCTCTGGTGAGAACTTTCCTCCCAATGGACCTGT
TATGCAGAAGAAGACACAGGGCTGGGAACCCAGCACTGAGCGTCTCTTTGCACGAGATGGAATGCTGATAGGAAACGAT
TATATGGCTCTGAAGTTGGAAGGAGGTGGTCACTATTTGTGTGAATTTAAATCTACTTACAAGGCAAAGAAGCCTGTGA
GGATGCCAGGGCGCCACGAGATTGACCGCAAACTGGATGTAACCAGTCACAACAGGGATTACACATCTGTTGAGCAGTG
TGAAATAGCCATTGCACGCCACTCTTTGCTCGGTTAA
mKeima (SEQ ID NO: 104)
MVSVIAKQMTYKVYMSGTVNGHYFEVEGDGKGKPYEGEQTVKLTVTKGGPLPFAWDILSPQLQYGSIPFTKYPEDIPDY
FKQSFPEGYTWERSMNFEDGAVCTVSNDSSIQGNCFIYNVKISGENFPPNGPVMQKKTQGWEPSTERLFARDGMLIGND
YMALKLEGGGHYLCEFKSTYKAKKPVRMPGRHEIDRKLDVTSHNRDYTSVEQCEIAIARHSLLG
mTurquoise 2 (SEQ ID NO: 105)
ATGGTGAGCAAGGGCGAGGAGCTGTTCACCGGGGTGGTGCCCATCCTGGTCGAGCTGGACGGCGACGTAAACGGCCACA
AGTTCAGCGTGTCCGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCCTGTCCTGGGGCGTGCAGTGCTTCGCCCGCTACCCCGACCACATG
AAGCAGCACGACTTCTTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAAGGGCATCGACTTCAA
GGAGGACGGCAACATCCTGGGGCACAAGCTGGAGTACAACTACTTTAGCGACAACGTCTATATCACCGCCGACAAGCAG
AAGAACGGCATCAAGGCCAACTTCAAGATCCGCCACAACATCGAGGACGGCGGCGTGCAGCTCGCCGACCACTACCAGC
AGAACACCCCCATCGGCGACGGCCCCGTGCTGCTGCCCGACAACCACTACCTGAGCACCCAGTCCAAGCTGAGCAAAGA
CCCCAACGAGAAGCGCGATCACATGGTCCTGCTGGAGTTCGTGACCGCCGCCGGGATCACTCTCGGCATGGACGAGCTG
TACAAGTAA
mTurquoise 2 (SEQ ID NO: 106)
MVSKGEELFTGVVPILVELDGDVNGHKFSVSGEGEGDATYGKLTLKFICTTGKLPVPWPTLVTTLSWGVQCFARYPDHM
KQHDFFKSAMPEGYVQERTIFFKDDGNYKTRAEVKFEGDTLVNRIELKGIDFKEDGNILGHKLEYNYFSDNVYITADKQ
KNGIKANFKIRHNIEDGGVQLADHYQQNTPIGDGPVLLPDNHYLSTQSKLSKDPNEKRDHMVLLEFVTAAGITLGMDEL
YK
Clover (SEQ ID NO: 107)
ATGGTGAGCAAGGGCGAGGAGCTGTTCACCGGGGTGGTGCCCATCCTGGTCGAGCTGGACGGCGACGTAAACGGCCACA
AGTTCAGCGTCCGCGGCGAGGGCGAGGGCGATGCCACCAACGGCAAGCTGACCCTGAAGTTCATCTGCACCACCGGCAA
GCTGCCCGTGCCCTGGCCCACCCTCGTGACCACCTTCGGCTACGGCGTGGCCTGCTTCAGCCGCTACCCCGACCACATG
AAGCAGCACGACTTCTTCAAGTCCGCCATGCCCGAAGGCTACGTCCAGGAGCGCACCATCTCTTTCAAGGACGACGGTA
CCTACAAGACCCGCGCCGAGGTGAAGTTCGAGGGCGACACCCTGGTGAACCGCATCGAGCTGAAGGGCATCGACTTCAA
GGAGGACGGCAACATCCTGGGGCACAAGCTGGAGTACAACTTCAACAGCCACAACGTCTATATCACGGCCGACAAGCAG
AAGAACGGCATCAAGGCTAACTTCAAGATCCGCCACAACGTTGAGGACGGCAGCGTGCAGCTCGCCGACCACTACCAGC
AGAACACCCCCATCGGCGACGGCCCCGTGCTGCTGCCCGACAACCACTACCTGAGCCATCAGTCCGCCCTGAGCAAAGA
CCCCAACGAGAAGCGCGATCACATGGTCCTGCTGGAGTTCGTGACCGCCGCCGGGATTACACATGGCATGGACGAGCTG
TACAAG
Clover (SEQ ID NO: 108)
MVSKGEELFTGVVPILVELDGDVNGHKFSVRGEGEGDATNGKLTLKFICTTGKLPVPWPTLVTTFGYGVACFSRYPDHM
KQHDFFKSAMPEGYVQERTISFKDDGTYKTRAEVKFEGDTLVNRIELKGIDFKEDGNILGHKLEYNFNSHNVYITADKQ
KNGIKANFKIRHNVEDGSVQLADHYQQNTPIGDGPVLLPDNHYLSHQSALSKDPNEKRDHMVLLEFVTAAGITHGMDEL
YK
mNeon-Green (SEQ ID NO: 109)
ATGGTGAGCAAGGGCGAGGAGGATAACATGGCCTCTCTCCCAGCGACACATGAGTTACACATCTTTGGCTCCATCAACG
GTGTGGACTTTGACATGGTGGGTCAGGGCACCGGCAATCCAAATGATGGTTATGAGGAGTTAAACCTGAAGTCCACCAA
GGGTGACCTCCAGTTCTCCCCCTGGATTCTGGTCCCTCATATCGGGTATGGCTTCCATCAGTACCTGCCCTACCCTGAC
GGGATGTCGCCTTTCCAGGCCGCCATGGTAGATGGCTCCGGATACCAAGTCCATCGCACAATGCAGTTTGAAGATGGTG
CCTCCCTTACTGTTAACTACCGCTACACCTACGAGGGAAGCCACATCAAAGGAGAGGCCCAGGTGAAGGGGACTGGTTT
CCCTGCTGACGGTCCTGTGATGACCAACTCGCTGACCGCTGCGGACTGGTGCAGGTCGAAGAAGACTTACCCCAACGAC
AAAACCATCATCAGTACCTTTAAGTGGAGTTACACCACTGGAAATGGCAAGCGCTACCGGAGCACTGCGCGGACCACCT
ACACCTTTGCCAAGCCAATGGCGGCTAACTATCTGAAGAACCAGCCGATGTACGTGTTCCGTAAGACGGAGCTCAAGCA
CTCCAAGACCGAGCTCAACTTCAAGGAGTGGCAAAAGGCCTTTACCGATGTGATGGGCATGGACGAGCTGTACAAGTAA
mNeon-Green (SEQ ID NO: 110)
MVSKGEEDNMASLPATHELHIFGSINGVDFDMVGQGTGNPNDGYEELNLKSTKGDLQFSPWILVPHIGYGFHQYLPYPD
GMSPFQAAMVDGSGYQVHRTMQFEDGASLTVNYRYTYEGSHIKGEAQVKGTGFPADGPVMTNSLTAADWCRSKKTYPND
KTIISTFKWSYTTGNGKRYRSTARTTYTFAKPMAANYLKNQPMYVFRKTELKHSKTELNFKEWQKAFTDVMGMDELYK
GGTGEL (SEQ ID NO: 111)
GGTGGS (SEQ ID NO: 112)
FKTRHN (SEQ ID NO: 113)
GGGGSGGGGS (SEQ ID NO: 114)
GKSSGSGSESKS (SEQ ID NO: 115),
GSTSGSGKSSEGKG (SEQ ID NO: 116)
GSTSGSGKSSEGSGSTKG (SEQ ID NO: 117)
GSTSGSGKPGSGEGSTKG (SEQ ID NO: 118)
EGKSSGSGSESKEF (SEQ ID NO: 119)
GGGGS (SEQ ID NO: 120)
GKSSGS (SEQ ID NO: 121)
GSESKS (SEQ ID NO: 122)
GSTSGSG (SEQ ID NO: 123)
KSSEGKG (SEQ ID NO: 124)
GSTSGSGKS (SEQ ID NO: 125)
SEGSGSTKG (SEQ ID NO: 126)
GSTSGSGKP (SEQ ID NO: 127)
GSGEGSTKG (SEQ ID NO: 128)
EGKSSGS (SEQ ID NO: 129)
GSESKEF (SEQ ID NO: 130)
mKOκ (SEQ ID NO: 131)
AGTGTGATTAAACCAGAGATGAAGATGAGGTACTACATGGACGGCTCCGTCAATGGGCATGAGTTCACAATTGAAGGTG
AAGGCACAGGCAGACCTTACGAGGGACATCAAGAGATGACACTACGCGTCACAATGGCCGAGGGCGGGCCAATGCCTTT
CGCGTTTGACTTAGTGTCACACGTGTTCTGTTACGGCCACAGAGTATTTACTAAATATCCAGAAGAGATACCAGACTAT
TTCAAACAAGCATTTCCTGAAGGCCTGTCATGGGAAAGGTCGTTGGAGTTCGAAGATGGTGGGTCCGCTTCAGTCAGTG
CGCATATAAGCCTTAGAGGAAACACCTTCTACCACAAATCCAAATTTACTGGGGTTAACTTTCCTGCCGATGGTCCTAT
CATGCAAAACCAAAGTGTTGATTGGGAGCCATCAACCGAGAAAATTACTGCCAGCGACGGAGTTCTGAAGGGTGATGTT
ACGATGTACCTAAAACTTGAAGGAGGCGGCAATCACAAATGCCAATTCAAGACTACTTACAAGGCGGCAAAAGAGATTC
TTGAAATGCCAGGAGACCATTACATCGGCCATCGCCTCGTCAGGAAAACCGAAGGCAACATTACTGAGCAGGTAGAAGA
TGCAGTAGCTCATTCCTAA
mKOκ (SEQ ID NO: 132)
ASVIKPEMKMRYYMDGSVNGHEFTIEGEGTGRPYEGHQEMTLRVTMAEGGPMPFAFDLVSHVFCYGHRVFTKYPEEIPD
YFKQAFPEGLSWERSLEFEDGGSASVSAHISLRGNTFYHKSKFTGVNFPADGPIMQNQSVDWEPSTEKITASDGVLKGD
VTMYLKLEGGGNHKCQFKTTYKAAKEILEMPGDHYIGHRLVRKTEGNITEQVEDAVAHS
List of Primers (SEQ ID NOS: 133-157)
AmLS_sfGFPcp_FW (SEQ ID NO: 133)
GTC CTC GTC GTG GTC GGT TCG AGA AAT TGT CCA ACG TGT ATA TTA CCG CGG
AmGS_sfGFPcp_FW (SEQ ID NO: 134)
GTC CTC GTC GTG GTC GGT TCG AGA AAG GTA GTA ACG TGT ATA TTA CCG CGG
AmFN_sfGFPcp_RV (SEQ ID NO: 135)
GCG CAG AGC AAT AGC GCG ACC ACC ATT AAA GTT ATA TTC CAG TTT ATG CCC
sfLS-LSSmO_FW (SEQ ID NO: 136)
GTC GTG GTC GGT TCG AGA AAT TGT CCA ACG TGT ATA TTA CCG CGG
sfLS-LSSmO_RV (SEQ ID NO: 137)
CCG CGG TAA TAT ACA CGT TGG ACA ATT TCT CGA ACC GAC CAC GAC
sfAmTrac-F138I_FW (SEQ ID NO: 138)
CTT CCT CTA CCA ATG GGC GAT CGC AAT CGC GGC CGC TGG
sfAmTrac-F138I_RV (SEQ ID NO: 139)
CCA GCG GCC GCG ATT GCG ATC GCC CAT TGG TAG AGG AAG
sfAmTrac-L255I_FW (SEQ ID NO: 140)
GTC TTA GGA ACC TTC CTC ATA TGG TTT GGA TGG
sfAmTrac-L255I_RV (SEQ ID NO: 141)
CCA TCC AAA CCA TAT GAG GAA GGT TCC TAA GAC
LS-cpsfGFP_FW (SEQ ID NO: 142)
GGC ATC ATC ATC ATC ATC ATA GCA GCG GCT TGT CCA ACG TGT ATA TTA CCG CGG
LS-cpsfGFP_RV (SEQ ID NO: 143)
CCG CGG TAA TAT ACA CGT TGG ACA AGC CGC TGC TAT GAT GAT GAT GAT GAT GCC
GS-cpsfGFP_FW (SEQ ID NO: 144)
GGC ATC ATC ATC ATC ATC ATA GCA GCG GCG GTA GTA ACG TGT ATA TTA CCG CGG
GS-cpsfGFP_RV (SEQ ID NO: 145)
CCG CGG TAA TAT ACA CGT TAC TAC CGC CGC TGC TAT GAT GAT GAT GAT GAT GCC
cpsfGFP-FN_FW (SEQ ID NO: 146)
GGG CAT AAA CTG GAA TAT AAC TTT AAT TAA CTC GAG GAT CCG GCT GC
cpsfGFP-FN_RV (SEQ ID NO: 147)
GCA GCC GGA TCC TCG AGT TAA TTA AAG TTA TAT TCC AGT TTA TGC CC
LSSmOr-pET15b_InF_1st_FW (SEQ ID NO: 148)
GGC ATC ATC ATC ATC ATC ATA GCA GCG GCA TGG TGA GCA AGG GCG AGG A
LSSmOr-pET15b_InF_1st_RV (SEQ ID NO: 149)
TTA CTT GTA CAG CTC GTC CAT GCC G
LSSmOr-pET15b_InF_2nd_FW (SEQ ID NO: 150)
agg aga tat aCC ATG GGG CAT CAT CAT CAT CAT CAT AGC AGC
LSSmOr-pET15b_InF_RV (SEQ ID NO: 151)
CAG CCG GAT CCT CGA GTT ACT TGT ACA GCT CGT CCA TGC CG
GCaMP6-EGFPcp-LSSmO-InF_FW (SEQ ID NO: 152)
GTACAAGGGCGGTACCATGGTGAGCAAGGGCGAGGA
GCaMP6-EGFPcp-LSSmO-InF_RV (SEQ ID NO: 153)
CACCATGCTCCCTCCCTTGTACAGCTCGTCCATGCC
sfGFPcp-XhoI-M13-InF_FW (SEQ ID NO: 154)
CTGAGCTCACTCGAGAACGTGTATATTACCGCGGAT
sfGFPcp-LP-CaM_RV (SEQ ID NO: 155)
TCAGTCAGTTGGTCCGGCAGGTTATATTCCAGTTTATGCCCC
CaM-LP-sfGFPcp_FW (SEQ ID NO: 156)
GGCATAAACTGGAATATAACCTGCCGGACCAACTGACTGA
CaM-pRSET-HindIII-InF_RV (SEQ ID NO: 157)
CAGCCGGATCAAGCTTCGAATTGC
GO-Matryoshka (LS-FN) T78H-DNA (SEQ ID NO: 163)
ttgtccAACGTGTATATTACCGCGGATAAACAGAAAAACGGCATTAAAGCGAACTTTcaCGTGCGCCATAACGTGGAAG
ATGGCAGCGTGCAGCTGGCGGATCATTATCAGCAGAACACCCCGATTGGCGATGGCCCGGTGCTGCTGCCGGATAACCA
TTATCTGAGCACCCAGACCAAGCTGAGCAAAGATCCGAACGAAAAACGCGATCACATGGTGCTGCTGGAATTTGTGACC
GCAGCGGGCATTACACACGGCATGGATGAACTGTATGGCGGCACCatggtgagcaagggcgaggagaataacatggcca
tcatcaaggagttcatgcgcttcaaggtgcgcatggagggctccgtgaacggccacgagttcgagatcgagggcgaggg
cgagggccgcccctacgagggctttcagaccgttaagctgaaggtgaccaagggtggccccctgcccttcgcctgggac
atcttgtcccctcagttcacctacggctccaaggcctacgtgaagcaccccgccgacatccccgactacctcaagctgt
ccttccccgagggcttcaagtgggagcgcgtgatgaacttcgaggacggcggcgtggtgaccgtgactcaggactcctc
cctgcaggacggcgagttcatctacaaggtgaagctgcgcggcaccaacttcccctccgacggccccgtaatgcagaag
aagaccatgggcatggaggcctcctccgagcggatgtaccccgaggacggcgccctgaagggcgaggacaagctcaggc
tgaagctgaaggacggcggccactacacctccgaggtcaagaccacctacaaggccaagaagcccgtgcagttgcccgg
cgcctacatcgtcgacatcaagttggacatcacctcccacaacgaggactacaccatcgtggaacagtacgaacgcgcc
gagggccgccactccaccggcggcatggacgagctgtacaagGGCGGCAGCGCGAGCCAGGGCGAAGAACTGTTTACCG
GCGTGGTGCCGATTCTGGTGGAACTGGATGGCGATGTGAACGGCCATAAATTTAGCGTGCGCGGCGAAGGCGAAGGCGA
TGCGACCATTGGCAAACTGACCCTGAAATTTATTTCCACCACCGGCAAACTACCGGTGCCGTGGCCGACCCTGGTGACC
ACCTTAACCTATGGCGTGCAGTGCTTTAGCCGCTATCCGGATCATATGAAACGCCATGATTTTTTTAAAAGCGCGATGC
CGGAAGGCTATGTGCAGGAACGCACCATTAGCTTTAAAGATGATGGCAAATATAAAACCCGCGCGGTGGTGAAATTTGA
AGGCGATACCCTGGTGAACCGCATTGAACTGAAAGGCACCGATTTTAAAGAAGATGGCAACATTCTGGGGCATAAACTG
GAATATAACtttaat
GO-Matryoshka (LS-FN) T78H-protein (SEQ ID NO: 164)
LSNVYITADKQKNGIKANFHVRHNVEDGSVQLADHYQQNTPIGDGPVLLPDNHYLSTQTKLSKDPNEKRDHMVLLEFVT
AAGITHGMDELYGGTMVSKGEENNMAIIKEFMRFKVRMEGSVNGHEFEIEGEGEGRPYEGFQTVKLKVTKGGPLPFAWD
ILSPQFTYGSKAYVKHPADIPDYLKLSFPEGFKWERVMNFEDGGVVTVTQDSSLQDGEFIYKVKLRGTNFPSDGPVMQK
KTMGMEASSERMYPEDGALKGEDKLRLKLKDGGHYTSEVKTTYKAKKPVQLPGAYIVDIKLDITSHNEDYTIVEQYERA
EGRHSTGGMDELYKGGSASQGEELFTGVVPILVELDGDVNGHKFSVRGEGEGDATIGKLTLKFISTTGKLPVPWPTLVT
TLTYGVQCFSRYPDHMKRHDFFKSAMPEGYVQERTISFKDDGKYKTRAVVKFEGDTLVNRIELKGTDFKEDGNILGHKL
EYNFN
AtAMT1: 3 DNA (SEQ ID NO: 165)
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAGGTGGTCGCGCT
ATTGCTCTGCGCGGCCACTCTGCCTCGCTAGTAGTCTTAGGAACCTTCCTCCTATGGTTTGGATGGTATGGTTTCAACC
CCGGTTCCTTCACTAAGATACTCGTTCCGTATAATTCTGGTTCCAACTACGGCCAATGGAGCGGAATCGGCCGTACAGC
GGTTAACACCACACTCTCAGGATGCACAGCAGCTCTAACCACACTCTTTGGTAAACGTCTCCTATCAGGCCACTGGAAC
GTAACGGACGTTTGCAACGGGTTACTCGGTGGGTTTGCGGCCATAACCGCAGGTTGCTCCGTCGTAGAGCCATGGGCAG
CGATTGTGTGCGGCTTCATGGCTTCTGTCGTCCTTATCGGATGCAACAAGCTCGCGGAGCTTGTACAATATGATGATCC
ACTCGAGGCAGCCCAACTACATGGAGGGTGTGGCGCGTGGGGGTTGATATTCGTAGGATTGTTTGCCAAAGAGAAGTAT
CTAAACGAGGTTTATGGCGCCACCCCGGGAAGGCCATATGGACTATTTATGGGCGGAGGAGGGAAGCTGTTGGGAGCAC
AATTGGTTCAAATACTTGTGATTGTAGGATGGGTTAGTGCCACAATGGGAACACTCTTCTTCATCCTCAAAAGGCTCAA
TCTGCTTAGGATCTCGGAGCAGCATGAAATGCAAGGGATGGATATGACACGTCACGGTGGCTTTGCTTATATCTACCAT
GATAATGATGATGAGTCTCATAGAGTGGATCCTGGATCTCCTTTCCCTCGATCAGCTACTCCTCCTCGCGTTTAA
AmTrac-LE (AmTrac)-DNA (SEQ ID NO: 166)
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAActcgagAACGTC
TATATCAaGGCCGACAAGCAGAAGAACGGCATCAAGGcGAACTTCAAGATCCGCCACAACATCGAGGACGGCgGCGTGC
AGCTCGCCtACCACTACCAGCAGAACACCCCCATCGGCGACGGCCCCGTGCTGCTGCCCGACAACCACTACCTGAGCgt
CCAGTCCaagCTGAGCAAAGACCCCAACGAGAAGCGCGATCACATGGTCCTGCTGGAGTTCGTGACCGCCGCCGGGATC
ACTCTCGGCATGGACGAGCTGTACAAGGGTGGTACCGGTGGATCTATGGTGAGCAAGGGCGAGGAGCTGTTCACCGGGG
TGGTGCCCATCCTGGTCGAGCTGGACGGCGACGTAAACGGCCACAAGTTCAGCGTGTCCGGCGAGGGCGAGGGCGATGC
CACCTACGGCAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCGCCATGCCCG
AAGGCTACaTCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCACAAGCTGGAG
TACAACtttaatGGTGGTCGCGCTATTGCTCTGCGCGGCCACTCTGCCTCGCTAGTAGTCTTAGGAACCTTCCTCCTAT
GGTTTGGATGGTATGGTTTCAACCCCGGTTCCTTCACTAAGATACTCGTTCCGTATAATTCTGGTTCCAACTACGGCCA
ATGGAGCGGAATCGGCCGTACAGCGGTTAACACCACACTCTCAGGATGCACAGCAGCTCTAACCACACTCTTTGGTAAA
CGTCTCCTATCAGGCCACTGGAACGTAACGGACGTTTGCAACGGGTTACTCGGTGGGTTTGCGGCCATAACCGCAGGTT
GCTCCGTCGTAGAGCCATGGGCAGCGATTGTGTGCGGCTTCATGGCTTCTGTCGTCCTTATCGGATGCAACAAGCTCGC
GGAGCTTGTACAATATGATGATCCACTCGAGGCAGCCCAACTACATGGAGGGTGTGGCGCGTGGGGGTTGATATTCGTA
GGATTGTTTGCCAAAGAGAAGTATCTAAACGAGGTTTATGGCGCCACCCCGGGAAGGCCATATGGACTATTTATGGGCG
GAGGAGGGAAGCTGTTGGGAGCACAATTGGTTCAAATACTTGTGATTGTAGGATGGGTTAGTGCCACAATGGGAACACT
CTTCTTCATCCTCAAAAGGCTCAATCTGCTTAGGATCTCGGAGCAGCATGAAATGCAAGGGATGGATATGACACGTCAC
GGTGGCTTTGCTTATATCTACCATGATAATGATGATGAGTCTCATAGAGTGGATCCTGGATCTCCTTTCCCTCGATCAG
CTACTCCTCCTCGCGTT
AmTrac-LE (AmTrac)-protein (SEQ ID NO: 167)
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKLENV
YIKADKQKNGIKANFKIRHNIEDGGVQLAYHYQQNTPIGDGPVLLPDNHYLSVQSKLSKDPNEKRDHMVLLEFVTAAGI
TLGMDELYKGGTGGSMVSKGEELFTGVVPILVELDGDVNGHKFSVSGEGEGDATYGKLTLKFICTTGKLPVPWPTLVTT
LTYGVQCFSRYPDHMKQHDFFKSAMPEGYIQERTIFFKDDGNYKTRAEVKFEGDTLVNRIELKGIDFKEDGNILGHKLE
YNFNGGRAIALRGHSASLVVLGTFLLWFGWYGFNPGSFTKILVPYNSGSNYGQWSGIGRTAVNTTLSGCTAALTTLFGK
RLLSGHWNVTDVCNGLLGGFAAITAGCSVVEPWAAIVCGFMASVVLIGCNKLAELVQYDDPLEAAQLHGGCGAWGLIFV
GLFAKEKYLNEVYGATPGRPYGLFMGGGGKLLGAQLVQILVIVGWVSATMGTLFFILKRLNLLRISEQHEMQGMDMTRH
GGFAYIYHDNDDESHRVDPGSPFPRSATPPRV
deAmTrac-CP-DNA SEQ ID NO: 168
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAtgtcccAACGTC
TATATCAaGGCCGACAAGCAGAAGAACGGCATCAAGGcGAACTTCAAGATCCGCCACAACATCGAGGACGGCgGCGTGC
AGCTCGCCtACCACTACCAGCAGAACACCCCCATCGGCGACGGCCCCGTGCTGCTGCCCGACAACCACTACCTGAGCgt
CCAGTCCaagCTGAGCAAAGACCCCAACGAGAAGCGCGATCACATGGTCCTGCTGGAGTTCGTGACCGCCGCCGGGATC
ACTCTCGGCATGGACGAGCTGTACAAGGGTGGTACCGGTGGATCTATGGTGAGCAAGGGCGAGGAGCTGTTCACCGGGG
TGGTGCCCATCCTGGTCGAGCTGGACGGCGACGTAAACGGCCACAAGTTCAGCGTGTCCGGCGAGGGCGAGGGCGATGC
CACCTACGGCAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCGCCATGCCCG
AAGGCTACaTCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCACAAGCTGGAG
TACAACtttaatGGTGGTCGCGCTATTGCTCTGCGCGGCCACTCTGCCTCGCTAGTAGTCTTAGGAACCTTCCTCCTAT
GGTTTGGATGGTATGGTTTCAACCCCGGTTCCTTCACTAAGATACTCGTTCCGTATAATTCTGGTTCCAACTACGGCCA
ATGGAGCGGAATCGGCCGTACAGCGGTTAACACCACACTCTCAGGATGCACAGCAGCTCTAACCACACTCTTTGGTAAA
CGTCTCCTATCAGGCCACTGGAACGTAACGGACGTTTGCAACGGGTTACTCGGTGGGTTTGCGGCCATAACCGCAGGTT
GCTCCGTCGTAGAGCCATGGGCAGCGATTGTGTGCGGCTTCATGGCTTCTGTCGTCCTTATCGGATGCAACAAGCTCGC
GGAGCTTGTACAATATGATGATCCACTCGAGGCAGCCCAACTACATGGAGGGTGTGGCGCGTGGGGGTTGATATTCGTA
GGATTGTTTGCCAAAGAGAAGTATCTAAACGAGGTTTATGGCGCCACCCCGGGAAGGCCATATGGACTATTTATGGGCG
GAGGAGGGAAGCTGTTGGGAGCACAATTGGTTCAAATACTTGTGATTGTAGGATGGGTTAGTGCCACAATGGGAACACT
CTTCTTCATCCTCAAAAGGCTCAATCTGCTTAGGATCTCGGAGCAGCATGAAATGCAAGGGATGGATATGACACGTCAC
GGTGGCTTTGCTTATATCTACCATGATAATGATGATGAGTCTCATAGAGTGGATCCTGGATCTCCTTTCCCTCGATCAG
CTACTCCTCCTCGCGTT
deAmTrac-CP-protein SEQ ID NO: 169
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKCPNV
YIKADKQKNGIKANFKIRHNIEDGGVQLAYHYQQNTPIGDGPVLLPDNHYLSVQSKLSKDPNEKRDHMVLLEFVTAAGI
TLGMDELYKGGTGGSMVSKGEELFTGVVPILVELDGDVNGHKFSVSGEGEGDATYGKLTLKFICTTGKLPVPWPTLVTT
LTYGVQCFSRYPDHMKQHDFFKSAMPEGYIQERTIFFKDDGNYKTRAEVKFEGDTLVNRIELKGIDFKEDGNILGHKLE
YNFNGGRAIALRGHSASLVVLGTFLLWFGWYGFNPGSFTKILVPYNSGSNYGQWSGIGRTAVNTTLSGCTAALTTLFGK
RLLSGHWNVTDVCNGLLGGFAAITAGCSVVEPWAAIVCGFMASVVLIGCNKLAELVQYDDPLEAAQLHGGCGAWGLIFV
GLFAKEKYLNEVYGATPGRPYGLFMGGGGKLLGAQLVQILVIVGWVSATMGTLFFILKRLNLLRISEQHEMQGMDMTRH
GGFAYIYHDNDDESHRVDPGSPFPRSATPPRV
deAmTrac-FP-protein SEQ ID NO: 170
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKFPNV
YIKADKQKNGIKANFKIRHNIEDGGVQLAYHYQQNTPIGDGPVLLPDNHYLSVQSKLSKDPNEKRDHMVLLEFVTAAGI
TLGMDELYKGGTGGSMVSKGEELFTGVVPILVELDGDVNGHKFSVSGEGEGDATYGKLTLKFICTTGKLPVPWPTLVTT
LTYGVQCFSRYPDHMKQHDFFKSAMPEGYIQERTIFFKDDGNYKTRAEVKFEGDTLVNRIELKGIDFKEDGNILGHKLE
YNFNGGRAIALRGHSASLVVLGTFLLWFGWYGFNPGSFTKILVPYNSGSNYGQWSGIGRTAVNTTLSGCTAALTTLFGK
RLLSGHWNVTDVCNGLLGGFAAITAGCSVVEPWAAIVCGFMASVVLIGCNKLAELVQYDDPLEAAQLHGGCGAWGLIFV
GLFAKEKYLNEVYGATPGRPYGLFMGGGGKLLGAQLVQILVIVGWVSATMGTLFFILKRLNLLRISEQHEMQGMDMTRH
GGFAYIYHDNDDESHRVDPGSPFPRSATPPRV
deAmTrac-FP-DNA SEQ ID NO: 171
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAtttcctAACGTC
TATATCAaGGCCGACAAGCAGAAGAACGGCATCAAGGcGAACTTCAAGATCCGCCACAACATCGAGGACGGCgGCGTGC
AGCTCGCCtACCACTACCAGCAGAACACCCCCATCGGCGACGGCCCCGTGCTGCTGCCCGACAACCACTACCTGAGCgt
CCAGTCCaagCTGAGCAAAGACCCCAACGAGAAGCGCGATCACATGGTCCTGCTGGAGTTCGTGACCGCCGCCGGGATC
ACTCTCGGCATGGACGAGCTGTACAAGGGTGGTACCGGTGGATCTATGGTGAGCAAGGGCGAGGAGCTGTTCACCGGGG
TGGTGCCCATCCTGGTCGAGCTGGACGGCGACGTAAACGGCCACAAGTTCAGCGTGTCCGGCGAGGGCGAGGGCGATGC
CACCTACGGCAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCGCCATGCCCG
AAGGCTACaTCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCACAAGCTGGAG
TACAACtttaatGGTGGTCGCGCTATTGCTCTGCGCGGCCACTCTGCCTCGCTAGTAGTCTTAGGAACCTTCCTCCTAT
GGTTTGGATGGTATGGTTTCAACCCCGGTTCCTTCACTAAGATACTCGTTCCGTATAATTCTGGTTCCAACTACGGCCA
ATGGAGCGGAATCGGCCGTACAGCGGTTAACACCACACTCTCAGGATGCACAGCAGCTCTAACCACACTCTTTGGTAAA
CGTCTCCTATCAGGCCACTGGAACGTAACGGACGTTTGCAACGGGTTACTCGGTGGGTTTGCGGCCATAACCGCAGGTT
GCTCCGTCGTAGAGCCATGGGCAGCGATTGTGTGCGGCTTCATGGCTTCTGTCGTCCTTATCGGATGCAACAAGCTCGC
GGAGCTTGTACAATATGATGATCCACTCGAGGCAGCCCAACTACATGGAGGGTGTGGCGCGTGGGGGTTGATATTCGTA
GGATTGTTTGCCAAAGAGAAGTATCTAAACGAGGTTTATGGCGCCACCCCGGGAAGGCCATATGGACTATTTATGGGCG
GAGGAGGGAAGCTGTTGGGAGCACAATTGGTTCAAATACTTGTGATTGTAGGATGGGTTAGTGCCACAATGGGAACACT
CTTCTTCATCCTCAAAAGGCTCAATCTGCTTAGGATCTCGGAGCAGCATGAAATGCAAGGGATGGATATGACACGTCAC
GGTGGCTTTGCTTATATCTACCATGATAATGATGATGAGTCTCATAGAGTGGATCCTGGATCTCCTTTCCCTCGATCAG
CTACTCCTCCTCGCGTT
sfAmTrac-LE-protein SEQ ID NO: 172
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKLENV
YITADKQKNGIKANFTVRHNVEDGSVQLADHYQQNTPIGDGPVLLPDNHYLSTQTKLSKDPNEKRDHMVLLEFVTAAGI
THGMDELYGGTGGSASQGEELFTGVVPILVELDGDVNGHKFSVRGEGEGDATIGKLTLKFISTTGKLPVPWPTLVTTLT
YGVQCFSRYPDHMKRHDFFKSAMPEGYVQERTISFKDDGKYKTRAVVKFEGDTLVNRIELKGTDFKEDGNILGHKLEYN
FNGGRAIALRGHSASLVVLGTFLLWFGWYGFNPGSFTKILVPYNSGSNYGQWSGIGRTAVNTTLSGCTAALTTLFGKRL
LSGHWNVTDVCNGLLGGFAAITAGCSVVEPWAAIVCGFMASVVLIGCNKLAELVQYDDPLEAAQLHGGCGAWGLIFVGL
FAKEKYLNEVYGATPGRPYGLFMGGGGKLLGAQLVQILVIVGWVSATMGTLFFILKRLNLLRISEQHEMQGMDMTRHGG
FAYIYHDNDDESHRVDPGSPFPRSATPPRV
sfAmTrac-LE-DNA SEQ ID NO: 173
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAActcgagAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCGGCGGCAGCGCGAGCCAGGGCGAAGAACTGTTTACCGGCGTGGTGC
CGATTCTGGTGGAACTGGATGGCGATGTGAACGGCCATAAATTTAGCGTGCGCGGCGAAGGCGAAGGCGATGCGACCAT
TGGCAAACTGACCCTGAAATTTATTTCCACCACCGGCAAACTACCGGTGCCGTGGCCGACCCTGGTGACCACCTTAACC
TATGGCGTGCAGTGCTTTAGCCGCTATCCGGATCATATGAAACGCCATGATTTTTTTAAAAGCGCGATGCCGGAAGGCT
ATGTGCAGGAACGCACCATTAGCTTTAAAGATGATGGCAAATATAAAACCCGCGCGGTGGTGAAATTTGAAGGCGATAC
CCTGGTGAACCGCATTGAACTGAAAGGCACCGATTTTAAAGAAGATGGCAACATTCTGGGGCATAAACTGGAATATAAC
tttaatGGTGGTCGCGCTATTGCTCTGCGCGGCCACTCTGCCTCGCTAGTAGTCTTAGGAACCTTCCTCCTATGGTTTG
GATGGTATGGTTTCAACCCCGGTTCCTTCACTAAGATACTCGTTCCGTATAATTCTGGTTCCAACTACGGCCAATGGAG
CGGAATCGGCCGTACAGCGGTTAACACCACACTCTCAGGATGCACAGCAGCTCTAACCACACTCTTTGGTAAACGTCTC
CTATCAGGCCACTGGAACGTAACGGACGTTTGCAACGGGTTACTCGGTGGGTTTGCGGCCATAACCGCAGGTTGCTCCG
TCGTAGAGCCATGGGCAGCGATTGTGTGCGGCTTCATGGCTTCTGTCGTCCTTATCGGATGCAACAAGCTCGCGGAGCT
TGTACAATATGATGATCCACTCGAGGCAGCCCAACTACATGGAGGGTGTGGCGCGTGGGGGTTGATATTCGTAGGATTG
TTTGCCAAAGAGAAGTATCTAAACGAGGTTTATGGCGCCACCCCGGGAAGGCCATATGGACTATTTATGGGCGGAGGAG
GGAAGCTGTTGGGAGCACAATTGGTTCAAATACTTGTGATTGTAGGATGGGTTAGTGCCACAATGGGAACACTCTTCTT
CATCCTCAAAAGGCTCAATCTGCTTAGGATCTCGGAGCAGCATGAAATGCAAGGGATGGATATGACACGTCACGGTGGC
TTTGCTTATATCTACCATGATAATGATGATGAGTCTCATAGAGTGGATCCTGGATCTCCTTTCCCTCGATCAGCTACTC
CTCCTCGCGTT
sfAmTrac-LS-protein SEQ ID NO: 174
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKLSNV
YITADKQKNGIKANFTVRHNVEDGSVQLADHYQQNTPIGDGPVLLPDNHYLSTQTKLSKDPNEKRDHMVLLEFVTAAGI
THGMDELYGGTGGSASQGEELFTGVVPILVELDGDVNGHKFSVRGEGEGDATIGKLTLKFISTTGKLPVPWPTLVTTLT
YGVQCFSRYPDHMKRHDFFKSAMPEGYVQERTISFKDDGKYKTRAVVKFEGDTLVNRIELKGTDFKEDGNILGHKLEYN
FNGGRAIALRGHSASLVVLGTFLLWFGWYGFNPGSFTKILVPYNSGSNYGQWSGIGRTAVNTTLSGCTAALTTLFGKRL
LSGHWNVTDVCNGLLGGFAAITAGCSVVEPWAAIVCGFMASVVLIGCNKLAELVQYDDPLEAAQLHGGCGAWGLIFVGL
FAKEKYLNEVYGATPGRPYGLFMGGGGKLLGAQLVQILVIVGWVSATMGTLFFILKRLNLLRISEQHEMQGMDMTRHGG
FAYIYHDNDDESHRVDPGSPFPRSATPPRV
sfAmTrac-LS-DNA SEQ ID NO: 175
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAttgtccAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCGGCGGCAGCGCGAGCCAGGGCGAAGAACTGTTTACCGGCGTGGTGC
CGATTCTGGTGGAACTGGATGGCGATGTGAACGGCCATAAATTTAGCGTGCGCGGCGAAGGCGAAGGCGATGCGACCAT
TGGCAAACTGACCCTGAAATTTATTTCCACCACCGGCAAACTACCGGTGCCGTGGCCGACCCTGGTGACCACCTTAACC
TATGGCGTGCAGTGCTTTAGCCGCTATCCGGATCATATGAAACGCCATGATTTTTTTAAAAGCGCGATGCCGGAAGGCT
ATGTGCAGGAACGCACCATTAGCTTTAAAGATGATGGCAAATATAAAACCCGCGCGGTGGTGAAATTTGAAGGCGATAC
CCTGGTGAACCGCATTGAACTGAAAGGCACCGATTTTAAAGAAGATGGCAACATTCTGGGGCATAAACTGGAATATAAC
TTTaatGGTGGTCGCGCTATTGCTCTGCGCGGCCACTCTGCCTCGCTAGTAGTCTTAGGAACCTTCCTCCTATGGTTTG
GATGGTATGGTTTCAACCCCGGTTCCTTCACTAAGATACTCGTTCCGTATAATTCTGGTTCCAACTACGGCCAATGGAG
CGGAATCGGCCGTACAGCGGTTAACACCACACTCTCAGGATGCACAGCAGCTCTAACCACACTCTTTGGTAAACGTCTC
CTATCAGGCCACTGGAACGTAACGGACGTTTGCAACGGGTTACTCGGTGGGTTTGCGGCCATAACCGCAGGTTGCTCCG
TCGTAGAGCCATGGGCAGCGATTGTGTGCGGCTTCATGGCTTCTGTCGTCCTTATCGGATGCAACAAGCTCGCGGAGCT
TGTACAATATGATGATCCACTCGAGGCAGCCCAACTACATGGAGGGTGTGGCGCGTGGGGGTTGATATTCGTAGGATTG
TTTGCCAAAGAGAAGTATCTAAACGAGGTTTATGGCGCCACCCCGGGAAGGCCATATGGACTATTTATGGGCGGAGGAG
GGAAGCTGTTGGGAGCACAATTGGTTCAAATACTTGTGATTGTAGGATGGGTTAGTGCCACAATGGGAACACTCTTCTT
CATCCTCAAAAGGCTCAATCTGCTTAGGATCTCGGAGCAGCATGAAATGCAAGGGATGGATATGACACGTCACGGTGGC
TTTGCTTATATCTACCATGATAATGATGATGAGTCTCATAGAGTGGATCCTGGATCTCCTTTCCCTCGATCAGCTACTC
CTCCTCGCGTT
sfAmTrac-GS-protein SEQ ID NO: 176
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKGSNV
YITADKQKNGIKANFTVRHNVEDGSVQLADHYQQNTPIGDGPVLLPDNHYLSTQTKLSKDPNEKRDHMVLLEFVTAAGI
THGMDELYGGTGGSASQGEELFTGVVPILVELDGDVNGHKFSVRGEGEGDATIGKLTLKFISTTGKLPVPWPTLVTTLT
YGVQCFSRYPDHMKRHDFFKSAMPEGYVQERTISFKDDGKYKTRAVVKFEGDTLVNRIELKGTDFKEDGNILGHKLEYN
FNGGRAIALRGHSASLVVLGTFLLWFGWYGFNPGSFTKILVPYNSGSNYGQWSGIGRTAVNTTLSGCTAALTTLFGKRL
LSGHWNVTDVCNGLLGGFAAITAGCSVVEPWAAIVCGFMASVVLIGCNKLAELVQYDDPLEAAQLHGGCGAWGLIFVGL
FAKEKYLNEVYGATPGRPYGLFMGGGGKLLGAQLVQILVIVGWVSATMGTLFFILKRLNLLRISEQHEMQGMDMTRHGG
FAYIYHDNDDESHRVDPGSPFPRSATPPRV
sfAmTrac-GS-DNA SEQ ID NO: 177
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAggtagtAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCGGCGGCAGCGCGAGCCAGGGCGAAGAACTGTTTACCGGCGTGGTGC
CGATTCTGGTGGAACTGGATGGCGATGTGAACGGCCATAAATTTAGCGTGCGCGGCGAAGGCGAAGGCGATGCGACCAT
TGGCAAACTGACCCTGAAATTTATTTCCACCACCGGCAAACTACCGGTGCCGTGGCCGACCCTGGTGACCACCTTAACC
TATGGCGTGCAGTGCTTTAGCCGCTATCCGGATCATATGAAACGCCATGATTTTTTTAAAAGCGCGATGCCGGAAGGCT
ATGTGCAGGAACGCACCATTAGCTTTAAAGATGATGGCAAATATAAAACCCGCGCGGTGGTGAAATTTGAAGGCGATAC
CCTGGTGAACCGCATTGAACTGAAAGGCACCGATTTTAAAGAAGATGGCAACATTCTGGGGCATAAACTGGAATATAAC
tttaatGGTGGTCGCGCTATTGCTCTGCGCGGCCACTCTGCCTCGCTAGTAGTCTTAGGAACCTTCCTCCTATGGTTTG
GATGGTATGGTTTCAACCCCGGTTCCTTCACTAAGATACTCGTTCCGTATAATTCTGGTTCCAACTACGGCCAATGGAG
CGGAATCGGCCGTACAGCGGTTAACACCACACTCTCAGGATGCACAGCAGCTCTAACCACACTCTTTGGTAAACGTCTC
CTATCAGGCCACTGGAACGTAACGGACGTTTGCAACGGGTTACTCGGTGGGTTTGCGGCCATAACCGCAGGTTGCTCCG
TCGTAGAGCCATGGGCAGCGATTGTGTGCGGCTTCATGGCTTCTGTCGTCCTTATCGGATGCAACAAGCTCGCGGAGCT
TGTACAATATGATGATCCACTCGAGGCAGCCCAACTACATGGAGGGTGTGGCGCGTGGGGGTTGATATTCGTAGGATTG
TTTGCCAAAGAGAAGTATCTAAACGAGGTTTATGGCGCCACCCCGGGAAGGCCATATGGACTATTTATGGGCGGAGGAG
GGAAGCTGTTGGGAGCACAATTGGTTCAAATACTTGTGATTGTAGGATGGGTTAGTGCCACAATGGGAACACTCTTCTT
CATCCTCAAAAGGCTCAATCTGCTTAGGATCTCGGAGCAGCATGAAATGCAAGGGATGGATATGACACGTCACGGTGGC
TTTGCTTATATCTACCATGATAATGATGATGAGTCTCATAGAGTGGATCCTGGATCTCCTTTCCCTCGATCAGCTACTC
CTCCTCGCGTT
AmTryoshka-GS-protein SEQ ID NO: 178
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKGSNV
YITADKQKNGIKANFTVRHNVEDGSVQLADHYQQNTPIGDGPVLLPDNHYLSTQTKLSKDPNEKRDHMVLLEFVTAAGI
THGMDELYGGTMVSKGEENNMAIIKEFMRFKVRMEGSVNGHEFEIEGEGEGRPYEGFQTVKLKVTKGGPLPFAWDILSP
QFTYGSKAYVKHPADIPDYLKLSFPEGFKWERVMNFEDGGVVTVTQDSSLQDGEFIYKVKLRGTNFPSDGPVMQKKTMG
MEASSERMYPEDGALKGEDKLRLKLKDGGHYTSEVKTTYKAKKPVQLPGAYIVDIKLDITSHNEDYTIVEQYERAEGRH
STGGMDELYKGGSASQGEELFTGVVPILVELDGDVNGHKFSVRGEGEGDATIGKLTLKFISTTGKLPVPWPTLVTTLTY
GVQCFSRYPDHMKRHDFFKSAMPEGYVQERTISFKDDGKYKTRAVVKFEGDTLVNRIELKGTDFKEDGNILGHKLEYNF
NGGRAIALRGHSASLVVLGTFLLWFGWYGFNPGSFTKILVPYNSGSNYGQWSGIGRTAVNTTLSGCTAALTTLFGKRLL
SGHWNVTDVCNGLLGGFAAITAGCSVVEPWAAIVCGFMASVVLIGCNKLAELVQYDDPLEAAQLHGGCGAWGLIFVGLF
AKEKYLNEVYGATPGRPYGLFMGGGGKLLGAQLVQILVIVGWVSATMGTLFFILKRLNLLRISEQHEMQGMDMTRHGGF
AYIYHDNDDESHRVDPGSPFPRSATPPRV
AmTryoshka-GS-DNA SEQ ID NO: 179
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAggtagtAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCatggtgagcaagggcgaggagaataacatggccatcatcaaggagt
tcatgcgcttcaaggtgcgcatggagggctccgtgaacggccacgagttcgagatcgagggcgagggcgagggccgccc
ctacgagggctttcagaccgttaagctgaaggtgaccaagggtggccccctgcccttcgcctgggacatcttgtcccct
cagttcacctacggctccaaggcctacgtgaagcaccccgccgacatccccgactacctcaagctgtccttccccgagg
gcttcaagtgggagcgcgtgatgaacttcgaggacggcggcgtggtgaccgtgactcaggactcctccctgcaggacgg
cgagttcatctacaaggtgaagctgcgcggcaccaacttcccctccgacggccccgtaatgcagaagaagaccatgggc
atggaggcctcctccgagcggatgtaccccgaggacggcgccctgaagggcgaggacaagctcaggctgaagctgaagg
acggcggccactacacctccgaggtcaagaccacctacaaggccaagaagcccgtgcagttgcccggcgcctacatcgt
cgacatcaagttggacatcacctcccacaacgaggactacaccatcgtggaacagtacgaacgcgccgagggccgccac
tccaccggcggcatggacgagctgtacaagGGCGGCAGCGCGAGCCAGGGCGAAGAACTGTTTACCGGCGTGGTGCCGA
TTCTGGTGGAACTGGATGGCGATGTGAACGGCCATAAATTTAGCGTGCGCGGCGAAGGCGAAGGCGATGCGACCATTGG
CAAACTGACCCTGAAATTTATTTCCACCACCGGCAAACTACCGGTGCCGTGGCCGACCCTGGTGACCACCTTAACCTAT
GGCGTGCAGTGCTTTAGCCGCTATCCGGATCATATGAAACGCCATGATTTTTTTAAAAGCGCGATGCCGGAAGGCTATG
TGCAGGAACGCACCATTAGCTTTAAAGATGATGGCAAATATAAAACCCGCGCGGTGGTGAAATTTGAAGGCGATACCCT
GGTGAACCGCATTGAACTGAAAGGCACCGATTTTAAAGAAGATGGCAACATTCTGGGGCATAAACTGGAATATAACttt
aatGGTGGTCGCGCTATTGCTCTGCGCGGCCACTCTGCCTCGCTAGTAGTCTTAGGAACCTTCCTCCTATGGTTTGGAT
GGTATGGTTTCAACCCCGGTTCCTTCACTAAGATACTCGTTCCGTATAATTCTGGTTCCAACTACGGCCAATGGAGCGG
AATCGGCCGTACAGCGGTTAACACCACACTCTCAGGATGCACAGCAGCTCTAACCACACTCTTTGGTAAACGTCTCCTA
TCAGGCCACTGGAACGTAACGGACGTTTGCAACGGGTTACTCGGTGGGTTTGCGGCCATAACCGCAGGTTGCTCCGTCG
TAGAGCCATGGGCAGCGATTGTGTGCGGCTTCATGGCTTCTGTCGTCCTTATCGGATGCAACAAGCTCGCGGAGCTTGT
ACAATATGATGATCCACTCGAGGCAGCCCAACTACATGGAGGGTGTGGCGCGTGGGGGTTGATATTCGTAGGATTGTTT
GCCAAAGAGAAGTATCTAAACGAGGTTTATGGCGCCACCCCGGGAAGGCCATATGGACTATTTATGGGCGGAGGAGGGA
AGCTGTTGGGAGCACAATTGGTTCAAATACTTGTGATTGTAGGATGGGTTAGTGCCACAATGGGAACACTCTTCTTCAT
CCTCAAAAGGCTCAATCTGCTTAGGATCTCGGAGCAGCATGAAATGCAAGGGATGGATATGACACGTCACGGTGGCTTT
GCTTATATCTACCATGATAATGATGATGAGTCTCATAGAGTGGATCCTGGATCTCCTTTCCCTCGATCAGCTACTCCTC
CTCGCGTT
AmTryoshka-GS-F138I-protein SEQ ID NO: 180
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAIAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKGSNV
YITADKQKNGIKANFTVRHNVEDGSVQLADHYQQNTPIGDGPVLLPDNHYLSTQTKLSKDPNEKRDHMVLLEFVTAAGI
THGMDELYGGTMVSKGEENNMAIIKEFMRFKVRMEGSVNGHEFEIEGEGEGRPYEGFQTVKLKVTKGGPLPFAWDILSP
QFTYGSKAYVKHPADIPDYLKLSFPEGFKWERVMNFEDGGVVTVTQDSSLQDGEFIYKVKLRGTNFPSDGPVMQKKTMG
MEASSERMYPEDGALKGEDKLRLKLKDGGHYTSEVKTTYKAKKPVQLPGAYIVDIKLDITSHNEDYTIVEQYERAEGRH
STGGMDELYKGGSASQGEELFTGVVPILVELDGDVNGHKFSVRGEGEGDATIGKLTLKFISTTGKLPVPWPTLVTTLTY
GVQCFSRYPDHMKRHDFFKSAMPEGYVQERTISFKDDGKYKTRAVVKFEGDTLVNRIELKGTDFKEDGNILGHKLEYNF
NGGRAIALRGHSASLVVLGTFLLWFGWYGFNPGSFTKILVPYNSGSNYGQWSGIGRTAVNTTLSGCTAALTTLFGKRLL
SGHWNVTDVCNGLLGGFAAITAGCSVVEPWAAIVCGFMASVVLIGCNKLAELVQYDDPLEAAQLHGGCGAWGLIFVGLF
AKEKYLNEVYGATPGRPYGLFMGGGGKLLGAQLVQILVIVGWVSATMGTLFFILKRLNLLRISEQHEMQGMDMTRHGGF
AYIYHDNDDESHRVDPGSPFPRSATPPRV
AmTryoshka-GS-F138I-DNA SEQ ID NO: 181
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGaTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAggtagtAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCatggtgagcaagggcgaggagaataacatggccatcatcaaggagt
tcatgcgcttcaaggtgcgcatggagggctccgtgaacggccacgagttcgagatcgagggcgagggcgagggccgccc
ctacgagggctttcagaccgttaagctgaaggtgaccaagggtggccccctgcccttcgcctgggacatcttgtcccct
cagttcacctacggctccaaggcctacgtgaagcaccccgccgacatccccgactacctcaagctgtccttccccgagg
gcttcaagtgggagcgcgtgatgaacttcgaggacggcggcgtggtgaccgtgactcaggactcctccctgcaggacgg
cgagttcatctacaaggtgaagctgcgcggcaccaacttcccctccgacggccccgtaatgcagaagaagaccatgggc
atggaggcctcctccgagcggatgtaccccgaggacggcgccctgaagggcgaggacaagctcaggctgaagctgaagg
acggcggccactacacctccgaggtcaagaccacctacaaggccaagaagcccgtgcagttgcccggcgcctacatcgt
cgacatcaagttggacatcacctcccacaacgaggactacaccatcgtggaacagtacgaacgcgccgagggccgccac
tccaccggcggcatggacgagctgtacaagGGCGGCAGCGCGAGCCAGGGCGAAGAACTGTTTACCGGCGTGGTGCCGA
TTCTGGTGGAACTGGATGGCGATGTGAACGGCCATAAATTTAGCGTGCGCGGCGAAGGCGAAGGCGATGCGACCATTGG
CAAACTGACCCTGAAATTTATTTCCACCACCGGCAAACTACCGGTGCCGTGGCCGACCCTGGTGACCACCTTAACCTAT
GGCGTGCAGTGCTTTAGCCGCTATCCGGATCATATGAAACGCCATGATTTTTTTAAAAGCGCGATGCCGGAAGGCTATG
TGCAGGAACGCACCATTAGCTTTAAAGATGATGGCAAATATAAAACCCGCGCGGTGGTGAAATTTGAAGGCGATACCCT
GGTGAACCGCATTGAACTGAAAGGCACCGATTTTAAAGAAGATGGCAACATTCTGGGGCATAAACTGGAATATAACttt
aatGGTGGTCGCGCTATTGCTCTGCGCGGCCACTCTGCCTCGCTAGTAGTCTTAGGAACCTTCCTCCTATGGTTTGGAT
GGTATGGTTTCAACCCCGGTTCCTTCACTAAGATACTCGTTCCGTATAATTCTGGTTCCAACTACGGCCAATGGAGCGG
AATCGGCCGTACAGCGGTTAACACCACACTCTCAGGATGCACAGCAGCTCTAACCACACTCTTTGGTAAACGTCTCCTA
TCAGGCCACTGGAACGTAACGGACGTTTGCAACGGGTTACTCGGTGGGTTTGCGGCCATAACCGCAGGTTGCTCCGTCG
TAGAGCCATGGGCAGCGATTGTGTGCGGCTTCATGGCTTCTGTCGTCCTTATCGGATGCAACAAGCTCGCGGAGCTTGT
ACAATATGATGATCCACTCGAGGCAGCCCAACTACATGGAGGGTGTGGCGCGTGGGGGTTGATATTCGTAGGATTGTTT
GCCAAAGAGAAGTATCTAAACGAGGTTTATGGCGCCACCCCGGGAAGGCCATATGGACTATTTATGGGCGGAGGAGGGA
AGCTGTTGGGAGCACAATTGGTTCAAATACTTGTGATTGTAGGATGGGTTAGTGCCACAATGGGAACACTCTTCTTCAT
CCTCAAAAGGCTCAATCTGCTTAGGATCTCGGAGCAGCATGAAATGCAAGGGATGGATATGACACGTCACGGTGGCTTT
GCTTATATCTACCATGATAATGATGATGAGTCTCATAGAGTGGATCCTGGATCTCCTTTCCCTCGATCAGCTACTCCTC
CTCGCGTT
AmTryoshka-GS-L255I-protein SEQ ID NO: 182
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKGSNV
YITADKQKNGIKANFTVRHNVEDGSVQLADHYQQNTPIGDGPVLLPDNHYLSTQTKLSKDPNEKRDHMVLLEFVTAAGI
THGMDELYGGTMVSKGEENNMAIIKEFMRFKVRMEGSVNGHEFEIEGEGEGRPYEGFQTVKLKVTKGGPLPFAWDILSP
QFTYGSKAYVKHPADIPDYLKLSFPEGFKWERVMNFEDGGVVTVTQDSSLQDGEFIYKVKLRGTNFPSDGPVMQKKTMG
MEASSERMYPEDGALKGEDKLRLKLKDGGHYTSEVKTTYKAKKPVQLPGAYIVDIKLDITSHNEDYTIVEQYERAEGRH
STGGMDELYKGGSASQGEELFTGVVPILVELDGDVNGHKFSVRGEGEGDATIGKLTLKFISTTGKLPVPWPTLVTTLTY
GVQCFSRYPDHMKRHDFFKSAMPEGYVQERTISFKDDGKYKTRAVVKFEGDTLVNRIELKGTDFKEDGNILGHKLEYNF
NGGRAIALRGHSASLVVLGTFLIWFGWYGFNPGSFTKILVPYNSGSNYGQWSGIGRTAVNTTLSGCTAALTTLFGKRLL
SGHWNVTDVCNGLLGGFAAITAGCSVVEPWAAIVCGFMASVVLIGCNKLAELVQYDDPLEAAQLHGGCGAWGLIFVGLF
AKEKYLNEVYGATPGRPYGLFMGGGGKLLGAQLVQILVIVGWVSATMGTLFFILKRLNLLRISEQHEMQGMDMTRHGGF
AYIYHDNDDESHRVDPGSPFPRSATPPRV
AmTryoshka-GS-L255I-DNA SEQ ID NO: 183
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAggtagtAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCatggtgagcaagggcgaggagaataacatggccatcatcaaggagt
tcatgcgcttcaaggtgcgcatggagggctccgtgaacggccacgagttcgagatcgagggcgagggcgagggccgccc
ctacgagggctttcagaccgttaagctgaaggtgaccaagggtggccccctgcccttcgcctgggacatcttgtcccct
cagttcacctacggctccaaggcctacgtgaagcaccccgccgacatccccgactacctcaagctgtccttccccgagg
gcttcaagtgggagcgcgtgatgaacttcgaggacggcggcgtggtgaccgtgactcaggactcctccctgcaggacgg
cgagttcatctacaaggtgaagctgcgcggcaccaacttcccctccgacggccccgtaatgcagaagaagaccatgggc
atggaggcctcctccgagcggatgtaccccgaggacggcgccctgaagggcgaggacaagctcaggctgaagctgaagg
acggcggccactacacctccgaggtcaagaccacctacaaggccaagaagcccgtgcagttgcccggcgcctacatcgt
cgacatcaagttggacatcacctcccacaacgaggactacaccatcgtggaacagtacgaacgcgccgagggccgccac
tccaccggcggcatggacgagctgtacaagGGCGGCAGCGCGAGCCAGGGCGAAGAACTGTTTACCGGCGTGGTGCCGA
TTCTGGTGGAACTGGATGGCGATGTGAACGGCCATAAATTTAGCGTGCGCGGCGAAGGCGAAGGCGATGCGACCATTGG
CAAACTGACCCTGAAATTTATTTCCACCACCGGCAAACTACCGGTGCCGTGGCCGACCCTGGTGACCACCTTAACCTAT
GGCGTGCAGTGCTTTAGCCGCTATCCGGATCATATGAAACGCCATGATTTTTTTAAAAGCGCGATGCCGGAAGGCTATG
TGCAGGAACGCACCATTAGCTTTAAAGATGATGGCAAATATAAAACCCGCGCGGTGGTGAAATTTGAAGGCGATACCCT
GGTGAACCGCATTGAACTGAAAGGCACCGATTTTAAAGAAGATGGCAACATTCTGGGGCATAAACTGGAATATAACttt
aatGGTGGTCGCGCTATTGCTCTGCGCGGCCACTCTGCCTCGCTAGTAGTCTTAGGAACCTTCCTCaTATGGTTTGGAT
GGTATGGTTTCAACCCCGGTTCCTTCACTAAGATACTCGTTCCGTATAATTCTGGTTCCAACTACGGCCAATGGAGCGG
AATCGGCCGTACAGCGGTTAACACCACACTCTCAGGATGCACAGCAGCTCTAACCACACTCTTTGGTAAACGTCTCCTA
TCAGGCCACTGGAACGTAACGGACGTTTGCAACGGGTTACTCGGTGGGTTTGCGGCCATAACCGCAGGTTGCTCCGTCG
TAGAGCCATGGGCAGCGATTGTGTGCGGCTTCATGGCTTCTGTCGTCCTTATCGGATGCAACAAGCTCGCGGAGCTTGT
ACAATATGATGATCCACTCGAGGCAGCCCAACTACATGGAGGGTGTGGCGCGTGGGGGTTGATATTCGTAGGATTGTTT
GCCAAAGAGAAGTATCTAAACGAGGTTTATGGCGCCACCCCGGGAAGGCCATATGGACTATTTATGGGCGGAGGAGGGA
AGCTGTTGGGAGCACAATTGGTTCAAATACTTGTGATTGTAGGATGGGTTAGTGCCACAATGGGAACACTCTTCTTCAT
CCTCAAAAGGCTCAATCTGCTTAGGATCTCGGAGCAGCATGAAATGCAAGGGATGGATATGACACGTCACGGTGGCTTT
GCTTATATCTACCATGATAATGATGATGAGTCTCATAGAGTGGATCCTGGATCTCCTTTCCCTCGATCAGCTACTCCTC
CTCGCGTT
AmTryoshka-LS-F138I-protein SEQ ID NO: 184
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAIAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKLSNV
YITADKQKNGIKANFTVRHNVEDGSVQLADHYQQNTPIGDGPVLLPDNHYLSTQTKLSKDPNEKRDHMVLLEFVTAAGI
THGMDELYGGTMVSKGEENNMAIIKEFMRFKVRMEGSVNGHEFEIEGEGEGRPYEGFQTVKLKVTKGGPLPFAWDILSP
QFTYGSKAYVKHPADIPDYLKLSFPEGFKWERVMNFEDGGVVTVTQDSSLQDGEFIYKVKLRGTNFPSDGPVMQKKTMG
MEASSERMYPEDGALKGEDKLRLKLKDGGHYTSEVKTTYKAKKPVQLPGAYIVDIKLDITSHNEDYTIVEQYERAEGRH
STGGMDELYKGGSASQGEELFTGVVPILVELDGDVNGHKFSVRGEGEGDATIGKLTLKFISTTGKLPVPWPTLVTTLTY
GVQCFSRYPDHMKRHDFFKSAMPEGYVQERTISFKDDGKYKTRAVVKFEGDTLVNRIELKGTDFKEDGNILGHKLEYNF
NGGRAIALRGHSASLVVLGTFLLWFGWYGFNPGSFTKILVPYNSGSNYGQWSGIGRTAVNTTLSGCTAALTTLFGKRLL
SGHWNVTDVCNGLLGGFAAITAGCSVVEPWAAIVCGFMASVVLIGCNKLAELVQYDDPLEAAQLHGGCGAWGLIFVGLF
AKEKYLNEVYGATPGRPYGLFMGGGGKLLGAQLVQILVIVGWVSATMGTLFFILKRLNLLRISEQHEMQGMDMTRHGGF
AYIYHDNDDESHRVDPGSPFPRSATPPRV
AmTryoshka-LS-F138I-DNA SEQ ID NO: 185
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGaTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAttgtccAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCatggtgagcaagggcgaggagaataacatggccatcatcaaggagt
tcatgcgcttcaaggtgcgcatggagggctccgtgaacggccacgagttcgagatcgagggcgagggcgagggccgccc
ctacgagggctttcagaccgttaagctgaaggtgaccaagggtggccccctgcccttcgcctgggacatcttgtcccct
cagttcacctacggctccaaggcctacgtgaagcaccccgccgacatccccgactacctcaagctgtccttccccgagg
gcttcaagtgggagcgcgtgatgaacttcgaggacggcggcgtggtgaccgtgactcaggactcctccctgcaggacgg
cgagttcatctacaaggtgaagctgcgcggcaccaacttcccctccgacggccccgtaatgcagaagaagaccatgggc
atggaggcctcctccgagcggatgtaccccgaggacggcgccctgaagggcgaggacaagctcaggctgaagctgaagg
acggcggccactacacctccgaggtcaagaccacctacaaggccaagaagcccgtgcagttgcccggcgcctacatcgt
cgacatcaagttggacatcacctcccacaacgaggactacaccatcgtggaacagtacgaacgcgccgagggccgccac
tccaccggcggcatggacgagctgtacaagGGCGGCAGCGCGAGCCAGGGCGAAGAACTGTTTACCGGCGTGGTGCCGA
TTCTGGTGGAACTGGATGGCGATGTGAACGGCCATAAATTTAGCGTGCGCGGCGAAGGCGAAGGCGATGCGACCATTGG
CAAACTGACCCTGAAATTTATTTCCACCACCGGCAAACTACCGGTGCCGTGGCCGACCCTGGTGACCACCTTAACCTAT
GGCGTGCAGTGCTTTAGCCGCTATCCGGATCATATGAAACGCCATGATTTTTTTAAAAGCGCGATGCCGGAAGGCTATG
TGCAGGAACGCACCATTAGCTTTAAAGATGATGGCAAATATAAAACCCGCGCGGTGGTGAAATTTGAAGGCGATACCCT
GGTGAACCGCATTGAACTGAAAGGCACCGATTTTAAAGAAGATGGCAACATTCTGGGGCATAAACTGGAATATAACttt
aatGGTGGTCGCGCTATTGCTCTGCGCGGCCACTCTGCCTCGCTAGTAGTCTTAGGAACCTTCCTCCTATGGTTTGGAT
GGTATGGTTTCAACCCCGGTTCCTTCACTAAGATACTCGTTCCGTATAATTCTGGTTCCAACTACGGCCAATGGAGCGG
AATCGGCCGTACAGCGGTTAACACCACACTCTCAGGATGCACAGCAGCTCTAACCACACTCTTTGGTAAACGTCTCCTA
TCAGGCCACTGGAACGTAACGGACGTTTGCAACGGGTTACTCGGTGGGTTTGCGGCCATAACCGCAGGTTGCTCCGTCG
TAGAGCCATGGGCAGCGATTGTGTGCGGCTTCATGGCTTCTGTCGTCCTTATCGGATGCAACAAGCTCGCGGAGCTTGT
ACAATATGATGATCCACTCGAGGCAGCCCAACTACATGGAGGGTGTGGCGCGTGGGGGTTGATATTCGTAGGATTGTTT
GCCAAAGAGAAGTATCTAAACGAGGTTTATGGCGCCACCCCGGGAAGGCCATATGGACTATTTATGGGCGGAGGAGGGA
AGCTGTTGGGAGCACAATTGGTTCAAATACTTGTGATTGTAGGATGGGTTAGTGCCACAATGGGAACACTCTTCTTCAT
CCTCAAAAGGCTCAATCTGCTTAGGATCTCGGAGCAGCATGAAATGCAAGGGATGGATATGACACGTCACGGTGGCTTT
GCTTATATCTACCATGATAATGATGATGAGTCTCATAGAGTGGATCCTGGATCTCCTTTCCCTCGATCAGCTACTCCTC
CTCGCGTT
AmTryoshka-LS-L255I-protein SEQ ID NO: 186
MSGAITCSAADLATLLGPNATAAADYICGQLGTVNNKFTDAAFAIDNTYLLFSAYLVFAMQLGFAMLCAGSVRAKNTMN
IMLTNVLDAAAGGLFYYLFGYAFAFGGSSEGFIGRHNFALRDFPTPTADYSFFLYQWAFAIAAAGITSGSIAERTQFVA
YLIYSSFLTGFVYPVVSHWFWSPDGWASPFRSADDRLFSTGAIDFAGSGVVHMVGGIAGLWGALIEGPRRGRFEKLSNV
YITADKQKNGIKANFTVRHNVEDGSVQLADHYQQNTPIGDGPVLLPDNHYLSTQTKLSKDPNEKRDHMVLLEFVTAAGI
THGMDELYGGTMVSKGEENNMAIIKEFMRFKVRMEGSVNGHEFEIEGEGEGRPYEGFQTVKLKVTKGGPLPFAWDILSP
QFTYGSKAYVKHPADIPDYLKLSFPEGFKWERVMNFEDGGVVTVTQDSSLQDGEFIYKVKLRGTNFPSDGPVMQKKTMG
MEASSERMYPEDGALKGEDKLRLKLKDGGHYTSEVKTTYKAKKPVQLPGAYIVDIKLDITSHNEDYTIVEQYERAEGRH
STGGMDELYKGGSASQGEELFTGVVPILVELDGDVNGHKFSVRGEGEGDATIGKLTLKFISTTGKLPVPWPTLVTTLTY
GVQCFSRYPDHMKRHDFFKSAMPEGYVQERTISFKDDGKYKTRAVVKFEGDTLVNRIELKGTDFKEDGNILGHKLEYNF
NGGRAIALRGHSASLVVLGTFLIWFGWYGFNPGSFTKILVPYNSGSNYGQWSGIGRTAVNTTLSGCTAALTTLFGKRLL
SGHWNVTDVCNGLLGGFAAITAGCSVVEPWAAIVCGFMASVVLIGCNKLAELVQYDDPLEAAQLHGGCGAWGLIFVGLF
AKEKYLNEVYGATPGRPYGLFMGGGGKLLGAQLVQILVIVGWVSATMGTLFFILKRLNLLRISEQHEMQGMDMTRHGGF
AYIYHDNDDESHRVDPGSPFPRSATPPRV
AmTryoshka-LS-L255I-DNA SEQ ID NO: 187
ATGTCAGGAGCAATAACATGCTCTGCGGCCGATCTCGCCACCCTACTTGGCCCCAACGCCACGGCGGCGGCCGACTACA
TTTGCGGCCAATTAGGCACCGTTAACAACAAGTTCACCGATGCAGCCTTCGCCATAGACAACACCTACCTCCTCTTCTC
TGCCTACCTTGTCTTCGCCATGCAGCTCGGCTTCGCTATGCTTTGTGCTGGTTCTGTTAGAGCCAAGAATACGATGAAC
ATCATGCTTACCAATGTCCTTGACGCTGCAGCCGGAGGACTCTTCTACTATCTCTTTGGTTACGCCTTTGCCTTTGGAG
GATCCTCCGAAGGGTTCATTGGAAGACACAACTTTGCTCTTAGAGACTTTCCGACTCCCACAGCTGATTACTCTTTCTT
CCTCTACCAATGGGCGTTCGCAATCGCGGCCGCTGGAATCACAAGTGGTTCGATCGCAGAGAGGACTCAGTTCGTGGCT
TACTTGATATACTCTTCTTTCTTAACCGGATTTGTTTACCCGGTTGTCTCTCACTGGTTTTGGTCCCCGGATGGATGGG
CCAGTCCCTTTCGTTCAGCGGATGATCGTTTGTTTAGCACCGGAGCCATTGACTTTGCTGGCTCCGGTGTTGTTCACAT
GGTTGGTGGCATAGCAGGTTTATGGGGTGCTCTTATTGAAGGTCCTCGTCGTGGTCGGTTCGAGAAAttgtccAACGTG
TATATTACCGCGGATAAACAGAAAAACGGCATTAAAGCGAACTTTACCGTGCGCCATAACGTGGAAGATGGCAGCGTGC
AGCTGGCGGATCATTATCAGCAGAACACCCCGATTGGCGATGGCCCGGTGCTGCTGCCGGATAACCATTATCTGAGCAC
CCAGACCAAGCTGAGCAAAGATCCGAACGAAAAACGCGATCACATGGTGCTGCTGGAATTTGTGACCGCAGCGGGCATT
ACACACGGCATGGATGAACTGTATGGCGGCACCatggtgagcaagggcgaggagaataacatggccatcatcaaggagt
tcatgcgcttcaaggtgcgcatggagggctccgtgaacggccacgagttcgagatcgagggcgagggcgagggccgccc
ctacgagggctttcagaccgttaagctgaaggtgaccaagggtggccccctgcccttcgcctgggacatcttgtcccct
cagttcacctacggctccaaggcctacgtgaagcaccccgccgacatccccgactacctcaagctgtccttccccgagg
gcttcaagtgggagcgcgtgatgaacttcgaggacggcggcgtggtgaccgtgactcaggactcctccctgcaggacgg
cgagttcatctacaaggtgaagctgcgcggcaccaacttcccctccgacggccccgtaatgcagaagaagaccatgggc
atggaggcctcctccgagcggatgtaccccgaggacggcgccctgaagggcgaggacaagctcaggctgaagctgaagg
acggcggccactacacctccgaggtcaagaccacctacaaggccaagaagcccgtgcagttgcccggcgcctacatcgt
cgacatcaagttggacatcacctcccacaacgaggactacaccatcgtggaacagtacgaacgcgccgagggccgccac
tccaccggcggcatggacgagctgtacaagGGCGGCAGCGCGAGCCAGGGCGAAGAACTGTTTACCGGCGTGGTGCCGA
TTCTGGTGGAACTGGATGGCGATGTGAACGGCCATAAATTTAGCGTGCGCGGCGAAGGCGAAGGCGATGCGACCATTGG
CAAACTGACCCTGAAATTTATTTCCACCACCGGCAAACTACCGGTGCCGTGGCCGACCCTGGTGACCACCTTAACCTAT
GGCGTGCAGTGCTTTAGCCGCTATCCGGATCATATGAAACGCCATGATTTTTTTAAAAGCGCGATGCCGGAAGGCTATG
TGCAGGAACGCACCATTAGCTTTAAAGATGATGGCAAATATAAAACCCGCGCGGTGGTGAAATTTGAAGGCGATACCCT
GGTGAACCGCATTGAACTGAAAGGCACCGATTTTAAAGAAGATGGCAACATTCTGGGGCATAAACTGGAATATAACttt
aatGGTGGTCGCGCTATTGCTCTGCGCGGCCACTCTGCCTCGCTAGTAGTCTTAGGAACCTTCCTCaTATGGTTTGGAT
GGTATGGTTTCAACCCCGGTTCCTTCACTAAGATACTCGTTCCGTATAATTCTGGTTCCAACTACGGCCAATGGAGCGG
AATCGGCCGTACAGCGGTTAACACCACACTCTCAGGATGCACAGCAGCTCTAACCACACTCTTTGGTAAACGTCTCCTA
TCAGGCCACTGGAACGTAACGGACGTTTGCAACGGGTTACTCGGTGGGTTTGCGGCCATAACCGCAGGTTGCTCCGTCG
TAGAGCCATGGGCAGCGATTGTGTGCGGCTTCATGGCTTCTGTCGTCCTTATCGGATGCAACAAGCTCGCGGAGCTTGT
ACAATATGATGATCCACTCGAGGCAGCCCAACTACATGGAGGGTGTGGCGCGTGGGGGTTGATATTCGTAGGATTGTTT
GCCAAAGAGAAGTATCTAAACGAGGTTTATGGCGCCACCCCGGGAAGGCCATATGGACTATTTATGGGCGGAGGAGGGA
AGCTGTTGGGAGCACAATTGGTTCAAATACTTGTGATTGTAGGATGGGTTAGTGCCACAATGGGAACACTCTTCTTCAT
CCTCAAAAGGCTCAATCTGCTTAGGATCTCGGAGCAGCATGAAATGCAAGGGATGGATATGACACGTCACGGTGGCTTT
GCTTATATCTACCATGATAATGATGATGAGTCTCATAGAGTGGATCCTGGATCTCCTTTCCCTCGATCAGCTACTCCTC
CTCGCGTT
GCaMP6s-protein SEQ ID NO: 188
SSRRKWNKTGHAVRAIGRLSSLENVYIKADKQKNGIKANFHIRHNIEDGGVQLAYHYQQNTPIGDGPVLLPDNHYLSVQ
SKLSKDPNEKRDHMVLLEFVTAAGITLGMDELYKGGTGGSMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYIQERTIFFKDDGNYKTRAEVKFEGD
TLVNRIELKGIDFKEDGNILGHKLEYNLPDQLTEEQIAEFKEAFSLFDKDGDGTITTKELGTVMRSLGQNPTEAELQDM
INEVDADGDGTIDFPEFLTMMARKMKYRDTEEEIREAFGVFDKDGNGYISAAELRHVMTNLGEKLTDEEVDEMIREADI
DGDGQVNYEEFVQMMTAK
GCaMP6s-DNA SEQ ID NO: 189
TCATCACGTCGTAAGTGGAATAAGACAGGTCACGCAGTCAGAGCTATAGGTCGGCTGAGCTCACTCGAGAACGTCTATA
TCAAGGCCGACAAGCAGAAGAACGGCATCAAGGCGAACTTCCACATCCGCCACAACATCGAGGACGGCGGCGTGCAGCT
CGCCTACCACTACCAGCAGAACACCCCCATCGGCGACGGCCCCGTGCTGCTGCCCGACAACCACTACCTGAGCGTGCAG
TCCAAACTTTCGAAAGACCCCAACGAGAAGCGCGATCACATGGTCCTGCTGGAGTTCGTGACCGCCGCCGGGATCACTC
TCGGCATGGACGAGCTGTACAAGGGCGGTACCGGAGGGAGCATGGTGAGCAAGGGCGAGGAGCTGTTCACCGGGGTGGT
GCCCATCCTGGTCGAGCTGGACGGCGACGTAAACGGCCACAAGTTCAGCGTGTCCGGCGAGGGTGAGGGCGATGCCACC
TACGGCAAGCTGACCCTGAAGTTCATCTGCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACGACTTCTTCAAGTCCGCCATGCCCGAAGG
CTACATCCAGGAGCGCACCATCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCTGGGGCACAAGCTGGAGTACA
ACCTGCCGGACCAACTGACTGAAGAGCAGATCGCAGAATTTAAAGAGGCTTTCTCCCTATTTGACAAGGACGGGGATGG
GACAATAACAACCAAGGAGCTGGGGACGGTGATGCGGTCTCTGGGGCAGAACCCCACAGAAGCAGAGCTGCAGGACATG
ATCAATGAAGTAGATGCCGACGGTGACGGCACAATCGACTTCCCTGAGTTCCTGACAATGATGGCAAGAAAAATGAAAT
ACAGGGACACGGAAGAAGAAATTAGAGAAGCGTTCGGTGTGTTTGATAAGGATGGCAATGGCTACATCAGTGCAGCAGA
GCTTCGCCACGTGATGACAAACCTTGGAGAGAAGTTAACAGATGAAGAGGTTGATGAAATGATCAGGGAAGCAGACATC
GATGGGGATGGTCAGGTAAACTACGAAGAGTTTGTACAAATGATGACAGCGAAGTGA
sfGaMP-protein SEQ ID NO: 190
SSRRKWNKTGHAVRAIGRLSSLENVYITADKQKNGIKANFTVRHNVEDGSVQLADHYQQNTPIGDGPVLLPDNHYLSTQ
TKLSKDPNEKRDHMVLLEFVTAAGITHGMDELYGGTGGSASQGEELFTGVVPILVELDGDVNGHKFSVRGEGEGDATIG
KLTLKFISTTGKLPVPWPTLVTTLTYGVQCFSRYPDHMKRHDFFKSAMPEGYVQERTISFKDDGKYKTRAVVKFEGDTL
VNRIELKGTDFKEDGNILGHKLEYNLPDQLTEEQIAEFKEAFSLFDKDGDGTITTKELGTVMRSLGQNPTEAELQDMIN
EVDADGDGTIDFPEFLTMMARKMKYRDTEEEIREAFGVFDKDGNGYISAAELRHVMTNLGEKLTDEEVDEMIREADIDG
DGQVNYEEFVQMMTAK
sfGaMP-DNA SEQ ID NO: 191
TCATCACGTCGTAAGTGGAATAAGACAGGTCACGCAGTCAGAGCTATAGGTCGGCTGAGCTCACTCGAGAACGTGTATA
TTACCGCGGATAAACAGAAAAACGGCATTAAAGCGAACTTTACCGTGCGCCATAACGTGGAAGATGGCAGCGTGCAGCT
GGCGGATCATTATCAGCAGAACACCCCGATTGGCGATGGCCCGGTGCTGCTGCCGGATAACCATTATCTGAGCACCCAG
ACCAAGCTGAGCAAAGATCCGAACGAAAAACGCGATCACATGGTGCTGCTGGAATTTGTGACCGCAGCGGGCATTACAC
ACGGCATGGATGAACTGTATGGCGGCACCGGCGGCAGCGCGAGCCAGGGCGAAGAACTGTTTACCGGCGTGGTGCCGAT
TCTGGTGGAACTGGATGGCGATGTGAACGGCCATAAATTTAGCGTGCGCGGCGAAGGCGAAGGCGATGCGACCATTGGC
AAACTGACCCTGAAATTTATTTCCACCACCGGCAAACTACCGGTGCCGTGGCCGACCCTGGTGACCACCTTAACCTATG
GCGTGCAGTGCTTTAGCCGCTATCCGGATCATATGAAACGCCATGATTTTTTTAAAAGCGCGATGCCGGAAGGCTATGT
GCAGGAACGCACCATTAGCTTTAAAGATGATGGCAAATATAAAACCCGCGCGGTGGTGAAATTTGAAGGCGATACCCTG
GTGAACCGCATTGAACTGAAAGGCACCGATTTTAAAGAAGATGGCAACATTCTGGGGCATAAACTGGAATATAACCTGC
CGGACCAACTGACTGAAGAGCAGATCGCAGAATTTAAAGAGGCTTTCTCCCTATTTGACAAGGACGGGGATGGGACAAT
AACAACCAAGGAGCTGGGGACGGTGATGCGGTCTCTGGGGCAGAACCCCACAGAAGCAGAGCTGCAGGACATGATCAAT
GAAGTAGATGCCGACGGTGACGGCACAATCGACTTCCCTGAGTTCCTGACAATGATGGCAAGAAAAATGAAATACAGGG
ACACGGAAGAAGAAATTAGAGAAGCGTTCGGTGTGTTTGATAAGGATGGCAATGGCTACATCAGTGCAGCAGAGCTTCG
CCACGTGATGACAAACCTTGGAGAGAAGTTAACAGATGAAGAGGTTGATGAAATGATCAGGGAAGCAGACATCGATGGG
GATGGTCAGGTAAACTACGAAGAGTTTGTACAAATGATGACAGCGAAGTGA
sfGaMP-T78H-protein SEQ ID NO: 192
SSRRKWNKTGHAVRAIGRLSSLENVYITADKQKNGIKANFHVRHNVEDGSVQLADHYQQNTPIGDGPVLLPDNHYLSTQ
TKLSKDPNEKRDHMVLLEFVTAAGITHGMDELYGGTGGSASQGEELFTGVVPILVELDGDVNGHKFSVRGEGEGDATIG
KLTLKFISTTGKLPVPWPTLVTTLTYGVQCFSRYPDHMKRHDFFKSAMPEGYVQERTISFKDDGKYKTRAVVKFEGDTL
VNRIELKGTDFKEDGNILGHKLEYNLPDQLTEEQIAEFKEAFSLFDKDGDGTITTKELGTVMRSLGQNPTEAELQDMIN
EVDADGDGTIDFPEFLTMMARKMKYRDTEEEIREAFGVFDKDGNGYISAAELRHVMTNLGEKLTDEEVDEMIREADIDG
DGQVNYEEFVQMMTAK
sfGaMP-T78H-DNA SEQ ID NO: 193
TCATCACGTCGTAAGTGGAATAAGACAGGTCACGCAGTCAGAGCTATAGGTCGGCTGAGCTCACTCGAGAACGTGTATA
TTACCGCGGATAAACAGAAAAACGGCATTAAAGCGAACTTTcaCGTGCGCCATAACGTGGAAGATGGCAGCGTGCAGCT
GGCGGATCATTATCAGCAGAACACCCCGATTGGCGATGGCCCGGTGCTGCTGCCGGATAACCATTATCTGAGCACCCAG
ACCAAGCTGAGCAAAGATCCGAACGAAAAACGCGATCACATGGTGCTGCTGGAATTTGTGACCGCAGCGGGCATTACAC
ACGGCATGGATGAACTGTATGGCGGCACCGGCGGCAGCGCGAGCCAGGGCGAAGAACTGTTTACCGGCGTGGTGCCGAT
TCTGGTGGAACTGGATGGCGATGTGAACGGCCATAAATTTAGCGTGCGCGGCGAAGGCGAAGGCGATGCGACCATTGGC
AAACTGACCCTGAAATTTATTTCCACCACCGGCAAACTACCGGTGCCGTGGCCGACCCTGGTGACCACCTTAACCTATG
GCGTGCAGTGCTTTAGCCGCTATCCGGATCATATGAAACGCCATGATTTTTTTAAAAGCGCGATGCCGGAAGGCTATGT
GCAGGAACGCACCATTAGCTTTAAAGATGATGGCAAATATAAAACCCGCGCGGTGGTGAAATTTGAAGGCGATACCCTG
GTGAACCGCATTGAACTGAAAGGCACCGATTTTAAAGAAGATGGCAACATTCTGGGGCATAAACTGGAATATAACCTGC
CGGACCAACTGACTGAAGAGCAGATCGCAGAATTTAAAGAGGCTTTCTCCCTATTTGACAAGGACGGGGATGGGACAAT
AACAACCAAGGAGCTGGGGACGGTGATGCGGTCTCTGGGGCAGAACCCCACAGAAGCAGAGCTGCAGGACATGATCAAT
GAAGTAGATGCCGACGGTGACGGCACAATCGACTTCCCTGAGTTCCTGACAATGATGGCAAGAAAAATGAAATACAGGG
ACACGGAAGAAGAAATTAGAGAAGCGTTCGGTGTGTTTGATAAGGATGGCAATGGCTACATCAGTGCAGCAGAGCTTCG
CCACGTGATGACAAACCTTGGAGAGAAGTTAACAGATGAAGAGGTTGATGAAATGATCAGGGAAGCAGACATCGATGGG
GATGGTCAGGTAAACTACGAAGAGTTTGTACAAATGATGACAGCGAAGTGA
MatryoshCaMP-protein SEQ ID NO: 194
SSRRKWNKTGHAVRAIGRLSSLENVYIKADKQKNGIKANFHIRHNIEDGGVQLAYHYQQNTPIGDGPVLLPDNHYLSVQ
SKLSKDPNEKRDHMVLLEFVTAAGITLGMDELYKGGTMVSKGEENNMAIIKEFMRFKVRMEGSVNGHEFEIEGEGEGRP
YEGFQTVKLKVTKGGPLPFAWDILSPQFTYGSKAYVKHPADIPDYLKLSFPEGFKWERVMNFEDGGVVTVTQDSSLQDG
EFIYKVKLRGTNFPSDGPVMQKKTMGMEASSERMYPEDGALKGEDKLRLKLKDGGHYTSEVKTTYKAKKPVQLPGAYIV
DIKLDITSHNEDYTIVEQYERAEGRHSTGGMDELYKGGSMVSKGEELFTGVVPILVELDGDVNGHKFSVSGEGEGDATY
GKLTLKFICTTGKLPVPWPTLVTTLTYGVQCFSRYPDHMKQHDFFKSAMPEGYIQERTIFFKDDGNYKTRAEVKFEGDT
LVNRIELKGIDFKEDGNILGHKLEYNLPDQLTEEQIAEFKEAFSLFDKDGDGTITTKELGTVMRSLGQNPTEAELQDMI
NEVDADGDGTIDFPEFLTMMARKMKYRDTEEEIREAFGVFDKDGNGYISAAELRHVMTNLGEKLTDEEVDEMIREADID
GDGQVNYEEFVQMMTAK
MatryoshCaMP-DNA SEQ ID NO: 195
TCATCACGTCGTAAGTGGAATAAGACAGGTCACGCAGTCAGAGCTATAGGTCGGCTGAGCTCACTCGAGAACGTCTATA
TCAAGGCCGACAAGCAGAAGAACGGCATCAAGGCGAACTTCCACATCCGCCACAACATCGAGGACGGCGGCGTGCAGCT
CGCCTACCACTACCAGCAGAACACCCCCATCGGCGACGGCCCCGTGCTGCTGCCCGACAACCACTACCTGAGCGTGCAG
TCCAAACTTTCGAAAGACCCCAACGAGAAGCGCGATCACATGGTCCTGCTGGAGTTCGTGACCGCCGCCGGGATCACTC
TCGGCATGGACGAGCTGTACAAGGGCGGTACCATGGTGAGCAAGGGCGAGGAgaataacatggccatcatcaaggagtt
catgcgcttcaaggtgcgcatggagggctccgtgaacggccacgagttcgagatcgagggcgagggcgagggccgcccc
tacgagggctttcagaccgttaagctgaaggtgaccaagggtggccccctgcccttcgcctgggacatcttgtcccctc
agttcacctacggctccaaggcctacgtgaagcaccccgccgacatccccgactacctcaagctgtccttccccgaggg
cttcaagtgggagcgcgtgatgaacttcgaggacggcggcgtggtgaccgtgactcaggactcctccctgcaggacggc
gagttcatctacaaggtgaagctgcgcggcaccaacttcccctccgacggccccgtaatgcagaagaagaccatgggca
tggaggcctcctccgagcggatgtaccccgaggacggcgccctgaagggcgaggacaagctcaggctgaagctgaagga
cggcggccactacacctccgaggtcaagaccacctacaaggccaagaagcccgtgcagttgcccggcgcctacatcgtc
gacatcaagttggacatcacctcccacaacgaggactacaccatcgtggaacagtacgaacgcgccgagggccgccact
ccaccggCGGCATGGACGAGCTGTACAAGGGAGGGAGCATGGTGAGCAAGGGCGAGGAGCTGTTCACCGGGGTGGTGCC
CATCCTGGTCGAGCTGGACGGCGACGTAAACGGCCACAAGTTCAGCGTGTCCGGCGAGGGTGAGGGCGATGCCACCTAC
GGCAAGCTGACCCTGAAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGCCATGCCCGAAGGCTA
CATCCAGGAGCGCACCATCTTCTTCAAGGACGACGGCAACTACAAGACCCGCGCCGAGGTGAAGTTCGAGGGCGACACC
CTGGTGAACCGCATCGAGCTGAAGGGCATCGACTTCAAGGAGGACGGCAACATCCTGGGGCACAAGCTGGAGTACAACC
TGCCGGACCAACTGACTGAAGAGCAGATCGCAGAATTTAAAGAGGCTTTCTCCCTATTTGACAAGGACGGGGATGGGAC
AATAACAACCAAGGAGCTGGGGACGGTGATGCGGTCTCTGGGGCAGAACCCCACAGAAGCAGAGCTGCAGGACATGATC
AATGAAGTAGATGCCGACGGTGACGGCACAATCGACTTCCCTGAGTTCCTGACAATGATGGCAAGAAAAATGAAATACA
GGGACACGGAAGAAGAAATTAGAGAAGCGTTCGGTGTGTTTGATAAGGATGGCAATGGCTACATCAGTGCAGCAGAGCT
TCGCCACGTGATGACAAACCTTGGAGAGAAGTTAACAGATGAAGAGGTTGATGAAATGATCAGGGAAGCAGACATCGAT
GGGGATGGTCAGGTAAACTACGAAGAGTTTGTACAAATGATGACAGCGAAGTGA
sfMatryoshCaMP-protein SEQ ID NO: 196
SSRRKWNKTGHAVRAIGRLSSLENVYITADKQKNGIKANFTVRHNVEDGSVQLADHYQQNTPIGDGPVLLPDNHYLSTQ
TKLSKDPNEKRDHMVLLEFVTAAGITHGMDELYGGTMVSKGEENNMAIIKEFMRFKVRMEGSVNGHEFEIEGEGEGRPY
EGFQTVKLKVTKGGPLPFAWDILSPQFTYGSKAYVKHPADIPDYLKLSFPEGFKWERVMNFEDGGVVTVTQDSSLQDGE
FIYKVKLRGTNFPSDGPVMQKKTMGMEASSERMYPEDGALKGEDKLRLKLKDGGHYTSEVKTTYKAKKPVQLPGAYIVD
IKLDITSHNEDYTIVEQYERAEGRHSTGGMDELYKGGSASQGEELFTGVVPILVELDGDVNGHKFSVRGEGEGDATIGK
LTLKFISTTGKLPVPWPTLVTTLTYGVQCFSRYPDHMKRHDFFKSAMPEGYVQERTISFKDDGKYKTRAVVKFEGDTLV
NRIELKGTDFKEDGNILGHKLEYNLPDQLTEEQIAEFKEAFSLFDKDGDGTITTKELGTVMRSLGQNPTEAELQDMINE
VDADGDGTIDFPEFLTMMARKMKYRDTEEEIREAFGVFDKDGNGYISAAELRHVMTNLGEKLTDEEVDEMIREADIDGD
GQVNYEEFVQMMTAK
sfMatryoshCaMP-DNA SEQ ID NO: 197
TCATCACGTCGTAAGTGGAATAAGACAGGTCACGCAGTCAGAGCTATAGGTCGGCTGAGCTCACTCGAGAACGTGTATA
TTACCGCGGATAAACAGAAAAACGGCATTAAAGCGAACTTTACCGTGCGCCATAACGTGGAAGATGGCAGCGTGCAGCT
GGCGGATCATTATCAGCAGAACACCCCGATTGGCGATGGCCCGGTGCTGCTGCCGGATAACCATTATCTGAGCACCCAG
ACCAAGCTGAGCAAAGATCCGAACGAAAAACGCGATCACATGGTGCTGCTGGAATTTGTGACCGCAGCGGGCATTACAC
ACGGCATGGATGAACTGTATGGCGGCACCatggtgagcaagggcgaggagaataacatggccatcatcaaggagttcat
gcgcttcaaggtgcgcatggagggctccgtgaacggccacgagttcgagatcgagggcgagggcgagggccgcccctac
gagggctttcagaccgttaagctgaaggtgaccaagggtggccccctgcccttcgcctgggacatcttgtcccctcagt
tcacctacggctccaaggcctacgtgaagcaccccgccgacatccccgactacctcaagctgtccttccccgagggctt
caagtgggagcgcgtgatgaacttcgaggacggcggcgtggtgaccgtgactcaggactcctccctgcaggacggcgag
ttcatctacaaggtgaagctgcgcggcaccaacttcccctccgacggccccgtaatgcagaagaagaccatgggcatgg
aggcctcctccgagcggatgtaccccgaggacggcgccctgaagggcgaggacaagctcaggctgaagctgaaggacgg
cggccactacacctccgaggtcaagaccacctacaaggccaagaagcccgtgcagttgcccggcgcctacatcgtcgac
atcaagttggacatcacctcccacaacgaggactacaccatcgtggaacagtacgaacgcgccgagggccgccactcca
ccggcggcatggacgagctgtacaagGGCGGCAGCGCGAGCCAGGGCGAAGAACTGTTTACCGGCGTGGTGCCGATTCT
GGTGGAACTGGATGGCGATGTGAACGGCCATAAATTTAGCGTGCGCGGCGAAGGCGAAGGCGATGCGACCATTGGCAAA
CTGACCCTGAAATTTATTTCCACCACCGGCAAACTACCGGTGCCGTGGCCGACCCTGGTGACCACCTTAACCTATGGCG
TGCAGTGCTTTAGCCGCTATCCGGATCATATGAAACGCCATGATTTTTTTAAAAGCGCGATGCCGGAAGGCTATGTGCA
GGAACGCACCATTAGCTTTAAAGATGATGGCAAATATAAAACCCGCGCGGTGGTGAAATTTGAAGGCGATACCCTGGTG
AACCGCATTGAACTGAAAGGCACCGATTTTAAAGAAGATGGCAACATTCTGGGGCATAAACTGGAATATAACCTGCCGG
ACCAACTGACTGAAGAGCAGATCGCAGAATTTAAAGAGGCTTTCTCCCTATTTGACAAGGACGGGGATGGGACAATAAC
AACCAAGGAGCTGGGGACGGTGATGCGGTCTCTGGGGCAGAACCCCACAGAAGCAGAGCTGCAGGACATGATCAATGAA
GTAGATGCCGACGGTGACGGCACAATCGACTTCCCTGAGTTCCTGACAATGATGGCAAGAAAAATGAAATACAGGGACA
CGGAAGAAGAAATTAGAGAAGCGTTCGGTGTGTTTGATAAGGATGGCAATGGCTACATCAGTGCAGCAGAGCTTCGCCA
CGTGATGACAAACCTTGGAGAGAAGTTAACAGATGAAGAGGTTGATGAAATGATCAGGGAAGCAGACATCGATGGGGAT
GGTCAGGTAAACTACGAAGAGTTTGTACAAATGATGACAGCGAAGTGA
sfMatryoshCaMP-T78H-protein SEQ ID NO: 198
SSRRKWNKTGHAVRAIGRLSSLENVYITADKQKNGIKANFHVRHNVEDGSVQLADHYQQNTPIGDGPVLLPDNHYLSTQ
TKLSKDPNEKRDHMVLLEFVTAAGITHGMDELYGGTMVSKGEENNMAIIKEFMRFKVRMEGSVNGHEFEIEGEGEGRPY
EGFQTVKLKVTKGGPLPFAWDILSPQFTYGSKAYVKHPADIPDYLKLSFPEGFKWERVMNFEDGGVVTVTQDSSLQDGE
FIYKVKLRGTNFPSDGPVMQKKTMGMEASSERMYPEDGALKGEDKLRLKLKDGGHYTSEVKTTYKAKKPVQLPGAYIVD
IKLDITSHNEDYTIVEQYERAEGRHSTGGMDELYKGGSASQGEELFTGVVPILVELDGDVNGHKFSVRGEGEGDATIGK
LTLKFISTTGKLPVPWPTLVTTLTYGVQCFSRYPDHMKRHDFFKSAMPEGYVQERTISFKDDGKYKTRAVVKFEGDTLV
NRIELKGTDFKEDGNILGHKLEYNLPDQLTEEQIAEFKEAFSLFDKDGDGTITTKELGTVMRSLGQNPTEAELQDMINE
VDADGDGTIDFPEFLTMMARKMKYRDTEEEIREAFGVFDKDGNGYISAAELRHVMTNLGEKLTDEEVDEMIREADIDGD
GQVNYEEFVQMMTAK
sfMatryoshCaMP-T78H-DNA SEQ ID NO: 199
TCATCACGTCGTAAGTGGAATAAGACAGGTCACGCAGTCAGAGCTATAGGTCGGCTGAGCTCACTCGAGAACGTGTATA
TTACCGCGGATAAACAGAAAAACGGCATTAAAGCGAACTTTcaCGTGCGCCATAACGTGGAAGATGGCAGCGTGCAGCT
GGCGGATCATTATCAGCAGAACACCCCGATTGGCGATGGCCCGGTGCTGCTGCCGGATAACCATTATCTGAGCACCCAG
ACCAAGCTGAGCAAAGATCCGAACGAAAAACGCGATCACATGGTGCTGCTGGAATTTGTGACCGCAGCGGGCATTACAC
ACGGCATGGATGAACTGTATGGCGGCACCatggtgagcaagggcgaggagaataacatggccatcatcaaggagttcat
gcgcttcaaggtgcgcatggagggctccgtgaacggccacgagttcgagatcgagggcgagggcgagggccgcccctac
gagggctttcagaccgttaagctgaaggtgaccaagggtggccccctgcccttcgcctgggacatcttgtcccctcagt
tcacctacggctccaaggcctacgtgaagcaccccgccgacatccccgactacctcaagctgtccttccccgagggctt
caagtgggagcgcgtgatgaacttcgaggacggcggcgtggtgaccgtgactcaggactcctccctgcaggacggcgag
ttcatctacaaggtgaagctgcgcggcaccaacttcccctccgacggccccgtaatgcagaagaagaccatgggcatgg
aggcctcctccgagcggatgtaccccgaggacggcgccctgaagggcgaggacaagctcaggctgaagctgaaggacgg
cggccactacacctccgaggtcaagaccacctacaaggccaagaagcccgtgcagttgcccggcgcctacatcgtcgac
atcaagttggacatcacctcccacaacgaggactacaccatcgtggaacagtacgaacgcgccgagggccgccactcca
ccggcggcatggacgagctgtacaagGGCGGCAGCGCGAGCCAGGGCGAAGAACTGTTTACCGGCGTGGTGCCGATTCT
GGTGGAACTGGATGGCGATGTGAACGGCCATAAATTTAGCGTGCGCGGCGAAGGCGAAGGCGATGCGACCATTGGCAAA
CTGACCCTGAAATTTATTTCCACCACCGGCAAACTACCGGTGCCGTGGCCGACCCTGGTGACCACCTTAACCTATGGCG
TGCAGTGCTTTAGCCGCTATCCGGATCATATGAAACGCCATGATTTTTTTAAAAGCGCGATGCCGGAAGGCTATGTGCA
GGAACGCACCATTAGCTTTAAAGATGATGGCAAATATAAAACCCGCGCGGTGGTGAAATTTGAAGGCGATACCCTGGTG
AACCGCATTGAACTGAAAGGCACCGATTTTAAAGAAGATGGCAACATTCTGGGGCATAAACTGGAATATAACCTGCCGG
ACCAACTGACTGAAGAGCAGATCGCAGAATTTAAAGAGGCTTTCTCCCTATTTGACAAGGACGGGGATGGGACAATAAC
AACCAAGGAGCTGGGGACGGTGATGCGGTCTCTGGGGCAGAACCCCACAGAAGCAGAGCTGCAGGACATGATCAATGAA
GTAGATGCCGACGGTGACGGCACAATCGACTTCCCTGAGTTCCTGACAATGATGGCAAGAAAAATGAAATACAGGGACA
CGGAAGAAGAAATTAGAGAAGCGTTCGGTGTGTTTGATAAGGATGGCAATGGCTACATCAGTGCAGCAGAGCTTCGCCA
CGTGATGACAAACCTTGGAGAGAAGTTAACAGATGAAGAGGTTGATGAAATGATCAGGGAAGCAGACATCGATGGGGAT
GGTCAGGTAAACTACGAAGAGTTTGTACAAATGATGACAGCGAAGTGA
GGT (SEQ ID NO: 200)
GEL (SEQ ID NO: 201)
GGT (SEQ ID NO: 202)
FKT (SEQ ID NO: 203)
RHN (SEQ ID NO: 204)
TSapphire DNA SEQ ID NO: 205
ATGGTGAGCAAGGGCGAGGAGCTGTTCACCGGGGTGGTGCCCATCCTGGTCGAGCTGGACGGCGACGTAAACGGCCACA
AGTTCAGCGTGTCCGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCTTCTCCTACGGCGTGATGGTGTTCGCCCGCTACCCCGACCACATG
AAGCAGCACGACTTCTTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAAGGGCATCGACTTCAA
GGAGGACGGCAACATCCTGGGGCACAAGCTGGAGTACAACTTCAACAGCCACAACGTCTATATCATGGCCGACAAGCAG
AAGAACGGCATCAAGGCCAACTTCAAGATCCGCCACAACATCGAGGACGGCGGCGTGCAGCTCGCCGACCACTACCAGC
AGAACACCCCCATCGGCGACGGCCCCGTGCTGCTGCCCGACAACCACTACCTGAGCATCCAGTCCAAGCTGAGCAAAGA
CCCCAACGAGAAGCGCGATCACATGGTCCTGCTGGAGTTCGTGACCGCCGCCGGGATCACTCTGGGCATGGACGAGCTG
TACAAGTAA
TSapphire protein SEQ ID NO: 206
MVSKGEELFTGVVPILVELDGDVNGHKFSVSGEGEGDATYGKLTLKFICTTGKLPVPWPTLVTTFSYGVMVFARYPDHM
KQHDFFKSAMPEGYVQERTIFFKDDGNYKTRAEVKFEGDTLVNRIELKGIDFKEDGNILGHKLEYNFNSHNVYIMADKQ
KNGIKANFKIRHNIEDGGVQLADHYQQNTPIGDGPVLLPDNHYLSIQSKLSKDPNEKRDHMVLLEFVTAAGITLGMDEL
YKf

1. A fluorescent polypeptide comprising a fusion of a circularly permuted, first fluorescent protein as a sensing domain and a second fluorescent protein as a reference domain, the first fluorescent protein comprising a first fluorescent moiety and the second fluorescent protein comprising a second fluorescent moiety, wherein the reference domain is nested within the sequence of the circularly permuted sensing domain, said first and second fluorescent proteins forming a single cassette which can be inserted into sites within a sensing protein of interest to generate a ratiometric biosensor.

2. A fluorescent polypeptide of claim 1 wherein the first fluorescent moiety and the second fluorescent moiety may be excited at a single wavelength and fluoresce at different or spectrally distinct wavelengths.

3. A fluorescent polypeptide of claim 1 wherein the circularly permuted, first fluorescent protein is optionally interrupted to form a free amino-terminus and a free carboxy-terminus of the fluorescent polypeptide that are different from the original amino-terminus of the first fluorescent protein and the original carboxy-terminus of the first fluorescent protein, respectively.

4. A fluorescent polypeptide of claim 1 wherein the circularly permuted, first fluorescent protein comprises an amino-terminus of the first fluorescent protein and a carboxy-terminus of the first fluorescent protein which are joined by the second fluorescent protein to form the fluorescent polypeptide, and an amino acid sequence connecting beta-strands of the first fluorescent protein is optionally interrupted to form a free amino-terminus and a free carboxy-terminus of the fluorescent polypeptide.

5. A fluorescent polypeptide of claim 3 wherein the amino-terminus of the first fluorescent protein is joined to the second fluorescent protein by a second linker and the carboxy-terminus of the first fluorescent protein is joined to the second fluorescent protein by a first linker, wherein said first linker and said second linker may be the same or different, said first linker and/or said second linker optionally comprising a sequence of amino acids.

6. A fluorescent polypeptide of claim 3 wherein the amino-terminus of the first fluorescent protein is joined to the carboxy-terminus of the second fluorescent protein and the carboxy-terminus of the first fluorescent protein is joined to the amino-terminus of the second fluorescent protein.

7. A fluorescent polypeptide of claim 5 wherein the sequence of amino acids comprises 1, 2, 3, 4, 5, 6, 7, 8, 9, 10, 11, 12, 13, 14, 15, 16, 17, 18, 19 or 20 amino acids.

8. A fluorescent polypeptide of claim 5 wherein the first linker and the second linker are flexible and comprise an amino acid sequence -Gly-Gly-.

9. A fluorescent polypeptide of claim 5 wherein the first linker and the second linker, if joined in the absence of the second fluorescent protein, would form an amino acid sequence comprising at least one of the following GGTGEL (SEQ ID NO:111), GGTGGS (SEQ ID NO:112), FKTRHN (SEQ ID NO:113), GGGGSGGGGS (SEQ ID NO:114), GKSSGSGSESKS (SEQ ID NO:115), GSTSGSGKSSEGKG (SEQ ID NO:116), GSTSGSGKSSEGSGSTKG (SEQ ID NO:117), GSTSGSGKPGSGEGSTKG (SEQ ID NO:118), or EGKSSGSGSESKEF (SEQ ID NO:119), or said first linker or second linker comprises amino acid sequence comprising GGT, GEL, GGS, FKT, RHN, GGGGS (SEQ ID NO:120), GKSSGS (SEQ ID NO:121), GSESKS (SEQ ID NO:122), GSTSGSG (SEQ ID NO:123), KSSEGKG (SEQ ID NO:124), GSTSGSGKS (SEQ ID NO:125), SEGSGSTKG (SEQ ID NO:126), GSTSGSGKP (SEQ ID NO:127), GSGEGSTKG (SEQ ID NO:128), EGKSSGS (SEQ ID NO:129), or GSESKEF (SEQ ID NO:130).

10. A fluorescent polypeptide of claim 3 wherein at least one of the free amino-terminus and the free carboxy-terminus comprise an amino acid sequence linker.

11. A fluorescent polypeptide of claim 1 wherein the first fluorescent protein is mCerulean (SEQ ID NO:2), GFP (SEQ ID NO:5), EGFP (SEQ ID NO:4), mVenus (SEQ ID NO:12), mT-Sapphire (SEQ ID NO:206), mCherry (SEQ ID NO:14), mKate (SEQ ID NO:16), mKate2 (SEQ ID NO:96), mRuby (SEQ ID NO:92), mRuby2 (SEQ ID NO:94) or mApple (SEQ ID NO:18).

12. A fluorescent polypeptide of claim 1 wherein the circularly permuted, first fluorescent protein is cpsfGFP (SEQ ID NO:7) or cpEGFP (SEQ ID NO:26).

13. A fluorescent polypeptide of claim 12 wherein the motif GGTGGS (SEQ ID NO:112) is GGTGEL (SEQ ID NO:111), FKTRHN (SEQ ID NO:113), GGGGSGGGGS (SEQ ID NO:114), GKSSGSGSESKS (SEQ ID NO:115), GSTSGSGKSSEGKG (SEQ ID NO:116), GSTSGSGKSSEGSGSTKG (SEQ ID NO:117), GSTSGSGKPGSGEGSTKG (SEQ ID NO:118), or EGKSSGSGSESKEF (SEQ ID NO:119), or one of GGT or GGS of the GGTGGS (SEQ ID NO:112) motif is GGT, GEL, GGS, FKT, RHN, GGGGS (SEQ ID NO:120), GKSSGS (SEQ ID NO:121), GSESKS (SEQ ID NO:122), GSTSGSG (SEQ ID NO:123), KSSEGKG (SEQ ID NO:124), GSTSGSGKS (SEQ ID NO:125), SEGSGSTKG (SEQ ID NO:126), GSTSGSGKP (SEQ ID NO:127), GSGEGSTKG (SEQ ID NO:128), EGKSSGS (SEQ ID NO:129), or GSESKEF (SEQ ID NO:130).

14. A fluorescent polypeptide of claim 1 wherein the second fluorescent protein is mVenus (SEQ ID NO:12), LSSmOrange (SEQ ID NO:20), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG.

15. A fluorescent polypeptide of claim 1 comprising GO-Matroshka-LS-FN (SEQ ID NO:30) or a sequence of GO-Matroshka-LS-FN wherein the LSSmOrange (SEQ ID NO:20) motif of SEQ ID NO:30 is mVenus (SEQ ID NO:12), mHoneydew (SEQ ID No:22), mBanana (SEQ ID NO:24), mOrange, dTomato (SEQ ID NO:84), tdTomato (SEQ ID NO:86), mTangerine (SEQ ID NO:88), mStrawberry (SEQ ID NO:90), mCherry (SEQ ID NO:14), mApple (SEQ ID NO:18), mRuby (SEQ ID NO:92), mRuby2 (SEQ ID NO:94), mKate2 (SEQ ID NO:96), mNeptune (SEQ ID No:98), TagRFP-T (SEQ ID NO:100), mBeRFP, LSS-mKate2 (SEQ ID NO:102), mKeima (SEQ ID NO:104), mKOκ (SEQ ID NO:132), mOrange, mTurquoise 2 (SEQ ID NO:106), Clover (SEQ ID NO:108), mNeon-Green (SEQ ID NO:110), or mUKG.

16. A fluorescent polypeptide of claim 3 wherein the circularly permuted, first fluorescent protein is optionally interrupted to form a free amino-terminus and a free carboxy-terminus of the fluorescent polypeptide that are different from the amino-terminus of the first fluorescent protein and the carboxy-terminus of the first fluorescent protein, respectively, said circularly permuted, first fluorescent protein being cpsfGFP (SEQ ID NO:7) or cpEGFP (SEQ ID NO:26) and said optional interruption being at any one of residues 128-148, residues 155-160, residues 168-176 or residues 227-229 of the first fluorescent protein.

17. A fluorescent polypeptide of claim 16 wherein the amino-terminal end is selected from E142, Y143, Y145, H148, D155, H169, E172, D173, A227 or I229 of the first fluorescent protein, and the carboxy-terminal end is selected from N144, N146, N144, N149, K162, K156, N170, I171, D173, E172, A227, or I229, of the first fluorescent protein.

18. A fluorescent sensor comprising a fluorescent polypeptide of claim 1.

19. A fluorescent sensor comprising a fluorescent polypeptide, said fluorescent polypeptide comprising a circularly permuted first fluorescent protein as a sensing domain and optionally a second fluorescent protein as a reference domain, said second fluorescent protein, when present, being nested within the sequence of the circularly permuted sensing domain so as to form a ratiometric fluorescent sensor.

20. A fluorescent sensor of claim 18 wherein the response of the sensor may be determined by ratiometric measurement of the fluorescence of the first moiety and the fluorescence of the second moiety upon excitation with said similar wavelength.

21. A fluorescent sensor of claim 18 further comprising a sensor polypeptide.

22. A fluorescent sensor of claim 21, wherein the sensor polypeptide is selected from the group consisting of calmodulin or binding fragment thereof, a calmodulin-related protein, recoverin, a nucleoside diphosphate or triphosphate binding protein, an inositol-1,4,5-triphospha-te receptor, a cyclic nucleotide receptor, a nitric oxide receptor, a growth factor receptor, a hormone receptor, a ligand-binding domain of a hormone receptor, a steroid hormone receptor, a ligand binding domain of a steroid hormone receptor, a cytokine receptor, a growth factor receptor, a neurotransmitter receptor, a ligand-gated channel, a voltage-gated channel, a protein kinase C, a domain of protein kinase C, a cGMP-dependent protein kinase, an inositol polyphosphate receptor, a phosphate receptor, a carbohydrate receptor, an SH2 domain, an SH3 domain, a PTB domain, an antibody, an antigen-binding site from an antibody, a single-chain antibody, a zinc-finger domain, a protein kinase substrate, a protease substrate, a phosphorylation domain, a redox sensitive loop, Perceval, CH-GECO 2.1, RCaMP, RGECO1, REX-GECO1, Flamindo2, FlincGs, DAG sensor, iGluSnFR, HyPer, Ins(1,3,4,5)P4, a maltose sensor, a membrane voltage sensor, peredox, sonar, protein phosphorylation, tandem fluorescent protein timers, rxRFP, a superoxide indicator, ASAP 1, a VSFP, or LOOn-GFP.

23. A fluorescent sensor of claim 22, wherein the sensor polypeptide is calmodulin or a calmodulin-related protein moiety.

24. A fluorescent sensor of claim 22 wherein the sensor polypeptide is a calmodulin-binding domain of skMLCKp, smMLCK, CaMKII, Caldesmon, Calspermin, phosphofructokinase calcineurin, phosphorylase kinase, Ca2+-ATPase 59 kDa PDE, 60 kDa PDE, nitric oxide synthase, type I adenylyl cyclase, Bordetella pertussis adenylyl cyclase, Neuromodulin, Spectrin, MARCKS, F52, beta-Adducin, HSP90a, HIV-1 gp160, BBMHBI, Dilute MHC, Mastoparan, Melittin, Glucagon, Secretin, VIP, GIP, or Model Peptide CBP2.

25. A fluorescent polypeptide of claim 1, wherein the circularly permuted, first fluorescent protein further comprises a localization sequence.

26. A fluorescent polypeptide of claim 1 wherein the circularly permuted, first fluorescent protein is capable of being made by a method of producing a circularly permuted fluorescent nucleic acid sequence, comprising: linking a nucleic acid sequence encoding a linker moiety to the 5′ nucleotide of a polynucleotide encoding the first fluorescent protein; circularizing the polynucleotide with the nucleic acid sequence encoding the linker sequence; and cleaving the circularized polynucleotide with a nuclease, wherein cleavage linearizes the circularized polynucleotide, and expressing the polynucleotide sequence.

27. A fluorescent sensor of claim 18, wherein the circularly permuted, first fluorescent protein is capable of being made by a method of producing a circularly permuted fluorescent nucleic acid sequence, comprising: linking a nucleic acid sequence encoding a linker moiety to the 5′ nucleotide of a polynucleotide encoding the first fluorescent protein; circularizing the polynucleotide with the nucleic acid sequence encoding the linker sequence; and cleaving the circularized polynucleotide with a nuclease, wherein cleavage linearizes the circularized polynucleotide, and expressing the polynucleotide sequence.

28. A fluorescent polypeptide of claim 26 wherein the first fluorescent protein is mCerulean (SEQ ID NO:2), GFP (SEQ ID NO:5), EGFP (SEQ ID NO:4), mVenus (SEQ ID NO:12), mT-Sapphire (SEQ ID NO:206), mCherry (SEQ ID NO:14), mKate (SEQ ID NO:16), or mApple (SEQ ID NO:18).

29. A fluorescent sensor of claim 27 wherein the first fluorescent protein is mCerulean (SEQ ID NO:2), GFP (SEQ ID NO:5), EGFP (SEQ ID NO:4), mVenus (SEQ ID NO:12), mT-Sapphire (SEQ ID NO:206), mCherry (SEQ ID NO:14), mKate (SEQ ID NO:16), or mApple (SEQ ID NO:18).

30. A nucleic acid sequence encoding a fluorescent polypeptide of claim 1.

31. A nucleic acid sequence encoding a fluorescent sensor of claim 18.

32. An expression vector containing the nucleic acid sequence of claim 30.

33. A transgenic non-human animal, plant, bacteria or fungi, isolated animal cell, or plant cell comprising a nucleic acid sequence of claim 30.

34. An expression vector comprising expression control sequences operatively linked to a nucleic acid sequence of claim 30.

35. A host cell transfected with an expression vector of claim 34.

36. The cell of claim 33, wherein the cell is a prokaryote.

37. The cell of claim 36, wherein the cell is E. coli.

38. The cell of claim 33, wherein the cell is a eukaryotic cell.

39. The cell of claim 38, wherein the cell is a yeast cell.

40. The cell of claim 33, wherein the cell is a mammalian cell.

41. A method of detecting the presence of an environmental parameter in a sample comprising contacting a sensor of claim 18 with the sample and determining a change in fluorescence of the sensor in response to the presence of the environmental parameter.

42. The method of claim 41 wherein the environmental parameter is the presence, absence or change in an ion, pH, calcium, ammonium, a hormone, a growth factor, a cytokine, a chemokine, a neurotransmitter, a ligand, a steroid, an insulin-like growth factor, insulin, somatostatin, glucagon, interleukins, IL-2, a transforming growth factor, TGF-α, TGF-β, a platelet-derived growth factor, an epidermal growth factor, a nerve growth factor, a fibroblast growth factor, interferon-gamma, GM-CSF, acetylcholine, a biogenic amine, an amino acid, ATP, a peptide, an opioid, a hypothalamic-releasing hormone, a neurohypophyseal hormone, a pituitary hormone, a tachykinin, a somatostatin, a gastrointestinal peptide, or a voltage.


SEQUENCE LISTING (206 sequences)

SEQ ID NO: 1 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 2 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSKLSKDPNEKRDHMVLLEFVTAAGITLGMDELYK

SEQ ID NO: 3 - protein, 238 aa, Artificial Sequence
  Description of Artificial Sequence Synthetic polypeptide
MSKGEELFTGVVPVLVELDGDVNGQKFSVSGEGEGDATYGKLTLNFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMGDKPKNGIKVNFKIRHNIKDGSVQLADHYQQNTPIGDGPVLLPDNHYLSTQSALSKDPNEKRDHMILLEFVTAARITHGMDELYK

SEQ ID NO: 4 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - protein, 238 aa, Artificial Sequence
  Description of Artificial Sequence Synthetic polypeptide
MSKGEELFTGVVPILVELDGDVNGQKFSVSGEGEGDATYGKLTLKFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDELYK

SEQ ID NO: 6 - DNA, 729 bp, Artificial Sequence
  Description of Artificial Sequence Synthetic polynucleotide
aacagccataacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacttt

SEQ ID NO: 7 - protein, 243 aa, Artificial Sequence
  Description of Artificial Sequence Synthetic polypeptide
NSHNVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNF

SEQ ID NO: 8 - protein, 243 aa, Artificial Sequence
  Description of Artificial Sequence Synthetic polypeptide
LSNVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 9 - DNA, 729 bp, Artificial Sequence
  Description of Artificial Sequence Synthetic polynucleotide
aacagccataacgtgtatattaccgcggataaacagaaaaacggcattaaagcgaactttcacgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacttt

SEQ ID NO: 10 - protein, 243 aa, Artificial Sequence
  Description of Artificial Sequence Synthetic polypeptide
NSHNVYITADKQKNGIKANFHVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNF

SEQ ID NO: 11 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 12 - protein, 239 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 13 - DNA, 711 bp, Artificial Sequence
  Description of Artificial Sequence Synthetic polynucleotide
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 14 - protein, 236 aa, Artificial Sequence
  Description of Artificial Sequence Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 15 - DNA, 714 bp, Artificial Sequence
  Description of Artificial Sequence Synthetic polynucleotide
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaagaccttcatcaaccacacccagggcatccccgacttctggaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaggggacatggccctgaagctcgtgggcgggggccacctgatctgcaacttgaagaccacatacagatccaagaaacccgctaagaacctcaagatgcccggcgtctactatgtggaccgcagactggaaagaatcaaggaggccgacaaagagacctacgtcgagcagcacgaggtggctgtggccagatactgcgacctccctagcaaactggggcacaagcttaattaa

SEQ ID NO: 16 - protein, 237 aa, Artificial Sequence
  Description of Artificial Sequence Synthetic polypeptide
MVSKGEELIKENMHMKLYMEGTVNNHHFKCTSEGEGKPYEGTQTMRIKVVEGGPLPFAFDILATSFMYGSKTFINHTQGIPDFWKQSFPEGFTWERVTTYEDGGVLTATQDTSLQDGCLIYNVKIRGVNFPSNGPVMQKKTLGWEANTEMLYPADGGLEGRGDMALKLVGGGHLICNLKTTYRSKKPAKNLKMPGVYYVDRRLERIKEADKETYVEQHEVAVARYCDLPSKLGHKLN

SEQ ID NO: 17 - DNA, 711 bp, Artificial Sequence
  Description of Artificial Sequence Synthetic polynucleotide
atggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaa

SEQ ID NO: 18 - protein, 236 aa, Artificial Sequence
  Description of Artificial Sequence Synthetic polypeptide
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 19 - DNA, 711 bp, Artificial Sequence
  Description of Artificial Sequence Synthetic polynucleotide
atggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagtaa

SEQ ID NO: 20 - protein, 236 aa, Artificial Sequence
  Description of Artificial Sequence Synthetic polypeptide
MVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYK

SEQ ID NO: 21 - DNA, 696 bp, Artificial Sequence
  Description of Artificial Sequence Synthetic polynucleotide
atggtgagcaagggcgaggaggtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtggggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggcggccaccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagattgacgggaagctggacatcacctcccacaacgaggactacaccatcgtggaacagtacgagcgcgccgagggcggccactccaccggcggcatggacgagctgtacaag

SEQ ID NO: 22 - protein, 232 aa, Artificial Sequence
  Description of Artificial Sequence Synthetic polypeptide
MVSKGEEVIKEFMRFKVRMEGSVNGHEFEIEGEGEGRPYEGTQTAKLKVTKGGPLPFAWDILSPQFMWGSKAYVKHPADIPDYLKLSFPEGFKWERVMNFEDGGVVTVTQDSSLQDGEFIYKVKLRGTNFPSDGPVMQKKTMGWAATTERMYPEDGALKGEIKMRLKLKDGGHYDAEVKTTYMAKKPVQLPGAYKIDGKLDITSHNEDYTIVEQYERAEGGHSTGGMDELYK

SEQ ID NO: 23 - DNA, 711 bp, Artificial Sequence
  Description of Artificial Sequence Synthetic polynucleotide
atggtgagcaagggcgaggagaataacatggccgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttctgttacggctccaaggcctacgtgaagcaccccactggtatccccgactacttcaagctgtccttccccgagggcttcaagtgggagcgcgtgatgaacttcgaggacggcggcgtggtgaccgtggctcaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacagcgccgagaccaagaccacctacaaggccaagaagcccgtgcagttgcccggcgcctacatagccggcgagaagatcgacatcacctcccacaatgaggactacactatcgtggaattgtacgagcgcgccgagggccgccactccaccggcggcatggacgagctgtacaagtag

SEQ ID NO: 24 - protein, 236 aa, Artificial Sequence
  Description of Artificial Sequence Synthetic polypeptide
MVSKGEENNMAVIKEFMRFKVRMEGSVNGHEFEIEGEGEGRPYEGTQTAKLKVTKGGPLPFAWDILSPQFCYGSKAYVKHPTGIPDYFKLSFPEGFKWERVMNFEDGGVVTVAQDSSLQDGEFIYKVKLRGTNFPSDGPVMQKKTMGWEASSERMYPEDGALKGEIKMRLKLKDGGHYSAETKTTYKAKKPVQLPGAYIAGEKIDITSHNEDYTIVELYERAEGRHSTGGMDELYK

SEQ ID NO: 25 - DNA, 723 bp, Artificial Sequence
  Description of Artificial Sequence Synthetic polynucleotide
aacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 26 - protein, 241 aa, Artificial Sequence
  Description of Artificial Sequence Synthetic polypeptide
NVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 27 - DNA, 729 bp, Artificial Sequence; identical to SEQ ID NO: 6
  Description of Artificial Sequence Synthetic polynucleotide

SEQ ID NO: 28 - protein, 243 aa, Artificial Sequence; identical to SEQ ID NO: 7
  Description of Artificial Sequence Synthetic polypeptide

SEQ ID NO: 29 - DNA, 1437 bp, Artificial Sequence
  Description of Artificial Sequence Synthetic polynucleotide
ttgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 30 - protein, 479 aa, Artificial Sequence
  Description of Artificial Sequence Synthetic polypeptide
LSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 31 - DNA, 708 bp, Artificial Sequence
  Description of Artificial Sequence Synthetic polynucleotide
atggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaag

SEQ ID NO: 32 - protein, 236 aa, Artificial Sequence; identical to SEQ ID NO: 20
  Description of Artificial Sequence Synthetic polypeptide

SEQ ID NO: 33 - DNA, 8434 bp, Artificial Sequence
  Description of Artificial Sequence Synthetic polynucleotide
actagtgaattcgcggccatcacaagtttgtacaaaaaagcaggctccatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtttaaacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcga

SEQ ID NO: 34 - protein, 498 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 35 - DNA, 9167 bp, Artificial Sequence
  Description of Artificial Sequence Synthetic polynucleotide
actagtgaattcgcggccatcacaagtttgtacaaaaaagcaggcttcatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcga

SEQ ID NO: 36 - protein, 245 aa, Artificial Sequence
  Description of Artificial Sequence Synthetic polypeptide
GSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFN

SEQ ID NO: 37 - DNA, 9167 bp, Artificial Sequence
  Description of Artificial Sequence Synthetic polynucleotide
actagtgaattcgcggccatcacaagtttgtacaaaaaagcaggcttcatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcga

SEQ ID NO: 38 - protein, 245 aa, Artificial Sequence
  Description of Artificial Sequence Synthetic polypeptide
LENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFN

SEQ ID NO: 39 - DNA, 9167 bp, Artificial Sequence
  Description of Artificial Sequence Synthetic polynucleotide
actagtgaattcgcggccatcacaagtttgtacaaaaaagcaggcttcatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcga

SEQ ID NO: 40 - protein, 245 aa, Artificial Sequence
  Description of Artificial Sequence Synthetic polypeptide
LSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFN

SEQ ID NO: 41 - DNA, 9869 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgatcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 42 - DNA, 1437 bp, Artificial Sequence; identical to SEQ ID NO: 29
  Description of Artificial Sequence Synthetic polynucleotide

SEQ ID NO: 43 - protein, 374 aa, Artificial Sequence
  Description of Artificial Sequence Synthetic polypeptide
LSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGK

SEQ ID NO: 44 - DNA, 9869 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcatatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 45 - DNA, 9161 bp, Artificial Sequence
  Description of Artificial Sequence Synthetic polynucleotide
actagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctcga

SEQ ID NO: 46 - DNA, 729 bp, Artificial Sequence
  Description of Artificial Sequence Synthetic polynucleotide
ggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 47 - protein, 243 aa, Artificial Sequence
  Description of Artificial Sequence Synthetic polypeptide
GSNVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 48 - DNA, 9863 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccgtgggtgaggatagcgtgctgatcaccgagaacatgcacatgaaactgtacatggagggcaccgtgaacgaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaaacctttatcaaccacacccagggcatccccgacttctttaagcagtccttccctgagggcttcacatgggagaggatcaccacatacgaagacgggggcgtgctgaccgctacccaggacaccagcctccagaacggctgcctcatctacaacgtcaagatcaacggggtgaacttcccatccaacggccctgtgatgcagaagaaaacactcggctgggaggccagcaccgagatgctgtaccccgctgacagcggcctgagaggccatagccagatggccctgaagctcgtgggcgggggctacctgcactgctccctcaagaccacatacagatccaagaaacccgctaagaacctcaagatgcccggcttctacttcgtggacaggagactggaaagaatcaaggaggccgacaaagagacctacgtcgagcagcacgagatggctgtggccaggtactgcgacctgcctagcaaactggggcacagcggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 49 - DNA, 720 bp, Artificial Sequence
  Description of Artificial Sequence Synthetic polynucleotide
ggcggcaccgtgggtgaggatagcgtgctgatcaccgagaacatgcacatgaaactgtacatggagggcaccgtgaacgaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaaacctttatcaaccacacccagggcatccccgacttctttaagcagtccttccctgagggcttcacatgggagaggatcaccacatacgaagacgggggcgtgctgaccgctacccaggacaccagcctccagaacggctgcctcatctacaacgtcaagatcaacggggtgaacttcccatccaacggccctgtgatgcagaagaaaacactcggctgggaggccagcaccgagatgctgtaccccgctgacagcggcctgagaggccatagccagatggccctgaagctcgtgggcgggggctacctgcactgctccctcaagaccacatacagatccaagaaacccgctaagaacctcaagatgcccggcttctacttcgtggacaggagactggaaagaatcaaggaggccgacaaagagacctacgtcgagcagcacgagatggctgtggccaggtactgcgacctgcctagcaaactggggcacagcggcggcagc

SEQ ID NO: 50 - protein, 240 aa, Artificial Sequence
  Description of Artificial Sequence Synthetic polypeptide
GGTVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRRLERIKEADKETYVEQHEMAVARYCDLPSKLGHSGGS

SEQ ID NO: 51 - DNA, 9869 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 52 - DNA, 1437 bp, Artificial Sequence
  Description of Artificial Sequence Synthetic polynucleotide
ggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 53 - protein, 479 aa, Artificial Sequence
  Description of Artificial Sequence Synthetic polypeptide
GSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 54 - DNA, 9869 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 55 - DNA, 726 bp, Artificial Sequence
  Description of Artificial Sequence Synthetic polynucleotide
ggcggcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcggcagc

SEQ ID NO: 56 - protein, 242 aa, Artificial Sequence
  Description of Artificial Sequence Synthetic polypeptide
GGTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGS

SEQ ID NO: 57 - DNA, 9854 bp, Artificial Sequence
  Description of Artificial Sequence Synthetic polynucleotide
gaactagtgaattcgcggccatcacaagtttgtacaaaaaagcaggctttatgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagaggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgttgacccagctttcttgtacaaagtggtgtagttaattcatgatggccgctgcaggtcgacctcgagggggggcccggtacccaattcgccctatagtgagtcgtattacgcgcggatccagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaattcttattcttgagtaactctttcctgtaggtcaggttgctttctcaggtatagcatgaggtcgctcttattgaccacacctctaccggcatgccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcatgtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccaagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcctc

SEQ ID NO: 58 - DNA, 711 bp, Artificial Sequence
  Description of Artificial Sequence Synthetic polynucleotide
ggcggcaccgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagaggcggcagc

SEQ ID NO: 59 - protein, 237 aa, Artificial Sequence
  Description of Artificial Sequence Synthetic polypeptide
GGTVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRGGS

SEQ ID NO: 60 - DNA, 1422 bp, Artificial Sequence
  Description of Artificial Sequence Synthetic polynucleotide
ggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagaggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 61 - protein, 474 aa, Artificial Sequence
  Description of Artificial Sequence Synthetic polypeptide
GSNVYITADKQKNGIKANFTVRHNVEDGSVQLADHYQQNTPIGDGPVLLPDNHYLSTQTKLSKDPNEKRDHMVLLEFVTAAGITHGMDELYGGTVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 62 - DNA, 1431 bp, Artificial Sequence
  Description of Artificial Sequence Synthetic polynucleotide
ggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccgtgggtgaggatagcgtgctgatcaccgagaacatgcacatgaaactgtacatggagggcaccgtgaacgaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaaacctttatcaaccacacccagggcatccccgacttctttaagcagtccttccctgagggcttcacatgggagaggatcaccacatacgaagacgggggcgtgctgaccgctacccaggacaccagcctccagaacggctgcctcatctacaacgtcaagatcaacggggtgaacttcccatccaacggccctgtgatgcagaagaaaacactcggctgggaggccagcaccgagatgctgtaccccgctgacagcggcctgagaggccatagccagatggccctgaagctcgtgggcgggggctacctgcactgctccctcaagaccacatacagatccaagaaacccgctaagaacctcaagatgcccggcttctacttcgtggacaggagactggaaagaatcaaggaggccgacaaagagacctacgtcgagcagcacgagatggctgtggccaggtactgcgacctgcctagcaaactggggcacagcggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 63 - protein, 477 aa, Artificial Sequence
  Description of Artificial Sequence Synthetic polypeptide
GSNVYITADKQKNGIKANFTVRHNVEDGSVQLADHYQQNTPIGDGPVLLPDNHYLSTQTKLSKDPNEKRDHMVLLEFVTAAGITHGMDELYGGTVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRRLERIKEADKETYVEQHEMAVARYCDLPSKLGHSGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 64 - protein, 972 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHRGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 65 - protein, 741 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 66 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLIWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 67 - protein, 743 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 68 - protein, 743 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 69 - protein, 743 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 70 - protein, 975 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRRLERIKEADKETYVEQHEMAVARYCDLPSKLGHSGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 71 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 72 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 73 - protein, 479 aa, Artificial Sequence
  Description of Artificial Sequence Synthetic polypeptide
GSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 74 - DNA, 4932 bp, Artificial Sequence
  Description of Artificial Sequence Synthetic polynucleotide
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgagcggccgcgactctagatcataatcagccataccacatttgtagaggttttacttgctttaaaaaacctcccacacctccccctgaacctgaaacataaaatgaatgcaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 75 - DNA, 1881 bp, Artificial Sequence
  Description of Artificial Sequence Synthetic polynucleotide
aacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 76 - protein, 626 aa, Artificial Sequence
  Description of Artificial Sequence Synthetic polypeptide
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 77 - protein, 479 aa, Artificial Sequence
  Description of Artificial Sequence Synthetic polypeptide
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLP

SEQ ID NO: 78 - DNA, 4218 bp, Artificial Sequence
  Description of Artificial Sequence Synthetic polynucleotide
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgagcggccgcgactctagatcataatcagccataccacatttgtagaggttttacttgctttaaaaaacctcccacacctccccctgaacctgaaacataaaatgaatgcaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 79 - DNA, 4926 bp, Artificial Sequence
  Description of Artificial Sequence Synthetic polynucleotide
gatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgagcggccgcgactctagatcataatcagccataccacatttgtagaggttttacttgctttaaaaaacctcccacacctccccctgaacctgaaacataaaatgaatgcaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 80 - DNA, 1881 bp, Artificial Sequence
  Description of Artificial Sequence Synthetic polynucleotide
ctcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 81 - protein, 626 aa, Artificial Sequence
  Description of Artificial Sequence Synthetic polypeptide
LENVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 82 - protein, 479 aa, Artificial Sequence
  Description of Artificial Sequence Synthetic polypeptide
LENVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLP

SEQ ID NO: 83 - DNA, 702 bp, Artificial Sequence
  Description of Artificial Sequence Synthetic polynucleotide
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

SEQ ID NO: 84 - protein, 234 aa, Artificial Sequence
  Description of Artificial Sequence Synthetic polypeptide
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 85 - DNA, 1431 bp, Artificial Sequence
  Description of Artificial Sequence Synthetic polynucleotide
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtag

SEQ ID NO: 86 - protein, 476 aa, Artificial Sequence
  Description of Artificial Sequence Synthetic polypeptide
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 87 - DNA, 696 bp, Artificial Sequence
  Description of Artificial Sequence Synthetic polynucleotide
atggtgagcaagggcgaggaggtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttctgttacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaattgtacgagcgcgccgagggccgccactccaccggcggcatggacgagctgtacaag

SEQ ID NO: 88 - protein, 232 aa, Artificial Sequence
  Description of Artificial Sequence Synthetic polypeptide
MVSKGEEVIKEFMRFKVRMEGSVNGHEFEIEGEGEGRPYEGTQTAKLKVTKGGPLPFAWDILSPQFCYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYMAKKPVQLPGAYKTDIKLDITSHNEDYTIVELYERAEGRHSTGGMDELYK

SEQ ID NO: 89 - DNA, 711 bp, Artificial Sequence
  Description of Artificial Sequence Synthetic polynucleotide
atggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtaa

SEQ ID NO: 90 - protein, 236 aa, Artificial Sequence
  Description of Artificial Sequence Synthetic polypeptide
MVSKGEENNMAIIKEFMRFKVRMEGSVNGHEFEIEGEGEGRPYEGTQTAKLKVTKGGPLPFAWDILTPNFTYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYKAKKPVQLPGAYIVGIKLDITSHNEDYTIVELYERAEGRHSTGGMDELYK

SEQ ID NO: 91 - DNA, 681 bp, Artificial Sequence
  Description of Artificial Sequence Synthetic polynucleotide
atgaacagcctgatcaaagaaaacatgcggatgaaggtggtgctggaaggcagcgtgaacggccaccagttcaagtgcaccggcgagggcgagggcaacccctacatgggcacccagaccatgcggatcaaagtgatcgagggcggacctctgcccttcgccttcgacatcctggccacatccttcatgtacggcagccggaccttcatcaagtaccccaagggcatccccgatttcttcaagcagagcttccccgagggcttcacctgggagagagtgaccagatacgaggacggcggcgtgatcaccgtgatgcaggacaccagcctggaagatggctgcctggtgtaccatgcccaggtcaggggcgtgaattttcccagcaacggcgccgtgatgcagaagaaaaccaagggctgggagcccaacaccgagatgatgtaccccgctgacggcggactgagaggctacacccacatggccctgaaggtggacggcggagggcacctgagctgcagcttcgtgaccacctaccgatccaagaaaaccgtgggcaacatcaagatgcccggcatccacgccgtggaccaccggctggaaaggctggaagagtccgacaacgagatgttcgtggtgcagcgggagcacgccgtggccaagttcgccggcctgggcggaggg

SEQ ID NO: 92 - protein, 227 aa, Artificial Sequence
  Description of Artificial Sequence Synthetic polypeptide
MNSLIKENMRMKVVLEGSVNGHQFKCTGEGEGNPYMGTQTMRIKVIEGGPLPFAFDILATSFMYGSRTFIKYPKGIPDFFKQSFPEGFTWERVTRYEDGGVITVMQDTSLEDGCLVYHAQVRGVNFPSNGAVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGG

SEQ ID NO: 93 - DNA, 711 bp, Artificial Sequence
  Description of Artificial Sequence Synthetic polynucleotide
atggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaag

SEQ ID NO: 94 - protein, 237 aa, Artificial Sequence
  Description of Artificial Sequence Synthetic polypeptide
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYK

SEQ ID NO: 95 - DNA, 699 bp, Artificial Sequence
  Description of Artificial Sequence Synthetic polynucleotide
atggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatga

SEQ ID NO: 96 - protein, 232 aa, Artificial Sequence
  Description of Artificial Sequence Synthetic polypeptide
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 97 - DNA, 735 bp, Artificial Sequence
  Description of Artificial Sequence Synthetic polynucleotide
atggtgtctaagggcgaagagctgattaaggagaacatgcacatgaagctgtacatggagggcaccgtgaacaaccaccacttcaagtgcacatccgagggcgaaggcaagccctacgagggcacccagaccggcagaatcaaggtggtcgagggcggccctctccccttcgccttcgacatcctggctacctgcttcatgtacggcagcaagaccttcatcaaccacacccagggcatccccgatttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccagtaccgagacgctgtaccccgctgacggcggcctggaaggcagatgcgacatggccctgaagctcgtgggcgggggccacctgatctgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggacgagctgtacaagtaa

SEQ ID NO: 98 - protein, 244 aa, Artificial Sequence
  Description of Artificial Sequence Synthetic polypeptide
MVSKGEELIKENMHMKLYMEGTVNNHHFKCTSEGEGKPYEGTQTGRIKVVEGGPLPFAFDILATCFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRCDMALKLVGGGHLICNLKTTYRSKKPAKNLKMPGVYFVDRRLERIKEADNETYVEQHEVAVARYCDLPSKLGHKLNGMDELYK

SEQ ID NO: 99 - DNA, 732 bp, Artificial Sequence
  Description of Artificial Sequence Synthetic polynucleotide
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaag

SEQ ID NO: 100 - protein, 244 aa, Artificial Sequence
  Description of Artificial Sequence Synthetic polypeptide
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYK

SEQ ID NO: 101 - DNA, 702 bp, Artificial Sequence
  Description of Artificial Sequence Synthetic polynucleotide
atgagcgagctgattaaggagaacatgcacatgaagctgtacatggaaggcaccgtgaacaaccaccacttcaagtgcacatccgagggcgaaggcaagccctacgagggcacccagaccatgagaatcaaggtggtcgagggcggccctctacccttcgccttcgacatcttggctaccagcttcatgtacggcagctacaccttcatcaaccacacccagggcatccccgacttctttaagcagtccttccctgagggcttcacatgggagagagtcaccacatacgaagacgggggcgtgctgaccgctacccaggacaccagcctccaggacggttgcctcatctacaacgtcaagatcagaggggtgaacttcacatccaacggccctgtgatgcagaagaaaacactcggctgggaggccggcaccgagatgctgtaccccgctgacggcggcctggaaggcagatctgacgacgccctgaagctcgtgggcgggggccacctgatctgcaacttgaagagcacatacagatccaagaaacccgctaagaatctcaaggtgcccggcgtctactatgtggaccgaagactggaaagaatcaaggaggccgacaaagagacctacgtcgagcagcacgaggtggctgtggccagatactgcgacctccctagcaaactggggcacaagcttaattaa

SEQ ID NO: 102 - protein, 233 aa, Artificial Sequence
  Description of Artificial Sequence Synthetic polypeptide
MSELIKENMHMKLYMEGTVNNHHFKCTSEGEGKPYEGTQTMRIKVVEGGPLPFAFDILATSFMYGSYTFINHTQGIPDFFKQSFPEGFTWERVTTYEDGGVLTATQDTSLQDGCLIYNVKIRGVNFTSNGPVMQKKTLGWEAGTEMLYPADGGLEGRSDDALKLVGGGHLICNLKSTYRSKKPAKNLKVPGVYYVDRRLERIKEADKETYVEQHEVAVARYCDLPSKLGHKLN

SEQ ID NO: 103 - DNA, 669 bp, Artificial Sequence
  Description of Artificial Sequence Synthetic polynucleotide
atggtgagtgtgatcgctaaacaaatgacctacaaggtttatatgtcaggcacggtcaatggacactactttgaggtcgaaggcgatggaaaaggaaagccttacgagggagagcagacagtaaagctcactgtcaccaagggtggacctctgccatttgcttgggatattttatcaccacagcttcagtacggaagcataccattcaccaagtaccctgaagacatccctgattatttcaagcagtcattccctgagggatatacatgggagaggagcatgaactttgaagatggtgcagtgtgtactgtcagcaatgattccagcatccaaggcaactgtttcatctacaatgtcaaaatctctggtgagaactttcctcccaatggacctgttatgcagaagaagacacagggctgggaacccagcactgagcgtctctttgcacgagatggaatgctgataggaaacgattatatggctctgaagttggaaggaggtggtcactatttgtgtgaatttaaatctacttacaaggcaaagaagcctgtgaggatgccagggcgccacgagattgaccgcaaactggatgtaaccagtcacaacagggattacacatctgttgagcagtgtgaaatagccattgcacgccactctttgctcggttaa

SEQ ID NO: 104 - protein, 222 aa, Artificial Sequence
  Description of Artificial Sequence Synthetic polypeptide
MVSVIAKQMTYKVYMSGTVNGHYFEVEGDGKGKPYEGEQTVKLTVTKGGPLPFAWDILSPQLQYGSIPFTKYPEDIPDYFKQSFPEGYTWERSMNFEDGAVCTVSNDSSIQGNCFIYNVKISGENFPPNGPVMQKKTQGWEPSTERLFARDGMLIGNDYMALKLEGGGHYLCEFKSTYKAKKPVRMPGRHEIDRKLDVTSHNRDYTSVEQCEIAIARHSLLG

SEQ ID NO: 105 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 106 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 107 - DNA, 717 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggctaacttcaagatccgccacaacgttgaggacggcagcgtgcagctcgccgaccactaccagcagaacacccccatcggcgacggccccgtgctgctgcccgacaaccactacctgagccatcagtccgccctgagcaaagaccccaacgagaagcgcgatcacatggtcctgctggagttcgtgaccgccgccgggattacacatggcatggacgagctgtacaag

SEQ ID NO: 108 - protein, 239 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSHQSALSKDPNEKRDHMVLLEFVTAAGITHGMDELYK

SEQ ID NO: 109 - DNA, 711 bp, Artificial Sequence
  Description of Artificial Sequence Synthetic polynucleotide
atggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaa

SEQ ID NO: 110 - protein, 236 aa, Artificial Sequence
  Description of Artificial Sequence Synthetic polypeptide
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 111 - protein, 6 aa, Artificial Sequence
  Description of Artificial Sequence Synthetic peptide
GGTGEL

SEQ ID NO: 112 - protein, 6 aa, Artificial Sequence
  Description of Artificial Sequence Synthetic peptide
GGTGGS

SEQ ID NO: 113 - protein, 6 aa, Artificial Sequence
  Description of Artificial Sequence Synthetic peptide
FKTRHN

SEQ ID NO: 114 - protein, 10 aa, Artificial Sequence
  Description of Artificial Sequence Synthetic peptide
GGGGSGGGGS

SEQ ID NO: 115 - protein, 12 aa, Artificial Sequence
  Description of Artificial Sequence Synthetic peptide
GKSSGSGSESKS

SEQ ID NO: 116 - protein, 14 aa, Artificial Sequence
  Description of Artificial Sequence Synthetic peptide
GSTSGSGKSSEGKG

SEQ ID NO: 117 - protein, 18 aa, Artificial Sequence
  Description of Artificial Sequence Synthetic peptide
GSTSGSGKSSEGSGSTKG

SEQ ID NO: 118 - protein, 18 aa, Artificial Sequence
  Description of Artificial Sequence Synthetic peptide
GSTSGSGKPGSGEGSTKG

SEQ ID NO: 119 - protein, 14 aa, Artificial Sequence
  Description of Artificial Sequence Synthetic peptide
EGKSSGSGSESKEF

SEQ ID NO: 120 - protein, 5 aa, Artificial Sequence
  Description of Artificial Sequence Synthetic peptide
GGGGS

SEQ ID NO: 121 - protein, 6 aa, Artificial Sequence
  Description of Artificial Sequence Synthetic peptide
GKSSGS

SEQ ID NO: 122 - protein, 6 aa, Artificial Sequence
  Description of Artificial Sequence Synthetic peptide
GSESKS

SEQ ID NO: 123 - protein, 7 aa, Artificial Sequence
  Description of Artificial Sequence Synthetic peptide
GSTSGSG

SEQ ID NO: 124 - protein, 7 aa, Artificial Sequence
  Description of Artificial Sequence Synthetic peptide
KSSEGKG

SEQ ID NO: 125 - protein, 9 aa, Artificial Sequence
  Description of Artificial Sequence Synthetic peptide
GSTSGSGKS

SEQ ID NO: 126 - protein, 9 aa, Artificial Sequence
  Description of Artificial Sequence Synthetic peptide
SEGSGSTKG

SEQ ID NO: 127 - protein, 9 aa, Artificial Sequence
  Description of Artificial Sequence Synthetic peptide
GSTSGSGKP

SEQ ID NO: 128 - protein, 9 aa, Artificial Sequence
  Description of Artificial Sequence Synthetic peptide
GSGEGSTKG

SEQ ID NO: 129 - protein, 7 aa, Artificial Sequence
  Description of Artificial Sequence Synthetic peptide
EGKSSGS

SEQ ID NO: 130 - protein, 7 aa, Artificial Sequence
  Description of Artificial Sequence Synthetic peptide
GSESKEF

SEQ ID NO: 131 - DNA, 651 bp, Artificial Sequence
  Description of Artificial Sequence Synthetic polynucleotide
agtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcggccatcgcctcgtcaggaaaaccgaaggcaacattactgagcaggtagaagatgcagtagctcattcctaa

SEQ ID NO: 132 - protein, 217 aa, Artificial Sequence
  Description of Artificial Sequence Synthetic polypeptide
ASVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITEQVEDAVAHS

SEQ ID NO: 133 - DNA, 51 bp, Artificial Sequence
  Description of Artificial Sequence Synthetic primer
gtcctcgtcgtggtcggttcgagaaattgtccaacgtgtatattaccgcgg

SEQ ID NO: 134 - DNA, 51 bp, Artificial Sequence
  Description of Artificial Sequence Synthetic primer
gtcctcgtcgtggtcggttcgagaaaggtagtaacgtgtatattaccgcgg

SEQ ID NO: 135 - DNA, 51 bp, Artificial Sequence
  Description of Artificial Sequence Synthetic primer
gcgcagagcaatagcgcgaccaccattaaagttatattccagtttatgccc

SEQ ID NO: 136 - DNA, 45 bp, Artificial Sequence
  Description of Artificial Sequence Synthetic primer
gtcgtggtcggttcgagaaattgtccaacgtgtatattaccgcgg

SEQ ID NO: 137 - DNA, 45 bp, Artificial Sequence
  Description of Artificial Sequence Synthetic primer
ccgcggtaatatacacgttggacaatttctcgaaccgaccacgac

SEQ ID NO: 138 - DNA, 39 bp, Artificial Sequence
  Description of Artificial Sequence Synthetic primer
cttcctctaccaatgggcgatcgcaatcgcggccgctgg

SEQ ID NO: 139 - DNA, 39 bp, Artificial Sequence
  Description of Artificial Sequence Synthetic primer
ccagcggccgcgattgcgatcgcccattggtagaggaag

SEQ ID NO: 140 - DNA, 33 bp, Artificial Sequence
  Description of Artificial Sequence Synthetic primer
gtcttaggaaccttcctcatatggtttggatgg

SEQ ID NO: 141 - DNA, 33 bp, Artificial Sequence
  Description of Artificial Sequence Synthetic primer
ccatccaaaccatatgaggaaggttcctaagac

SEQ ID NO: 142 - DNA, 54 bp, Artificial Sequence
  Description of Artificial Sequence Synthetic primer
ggcatcatcatcatcatcatagcagcggcttgtccaacgtgtatattaccgcgg

SEQ ID NO: 143 - DNA, 54 bp, Artificial Sequence
  Description of Artificial Sequence Synthetic primer
ccgcggtaatatacacgttggacaagccgctgctatgatgatgatgatgatgcc

SEQ ID NO: 144 - DNA, 54 bp, Artificial Sequence
  Description of Artificial Sequence Synthetic primer
ggcatcatcatcatcatcatagcagcggcggtagtaacgtgtatattaccgcgg

SEQ ID NO: 145 - DNA, 54 bp, Artificial Sequence
  Description of Artificial Sequence Synthetic primer
ccgcggtaatatacacgttactaccgccgctgctatgatgatgatgatgatgcc

SEQ ID NO: 146 - DNA, 47 bp, Artificial Sequence
  Description of Artificial Sequence Synthetic primer
gggcataaactggaatataactttaattaactcgaggatccggctgc

SEQ ID NO: 147 - DNA, 47 bp, Artificial Sequence
  Description of Artificial Sequence Synthetic primer
gcagccggatcctcgagttaattaaagttatattccagtttatgccc

SEQ ID NO: 148 - DNA, 49 bp, Artificial Sequence
  Description of Artificial Sequence Synthetic primer
ggcatcatcatcatcatcatagcagcggcatggtgagcaagggcgagga

SEQ ID NO: 149 - DNA, 25 bp, Artificial Sequence
  Description of Artificial Sequence Synthetic primer
ttacttgtacagctcgtccatgccg

SEQ ID NO: 150 - DNA, 42 bp, Artificial Sequence
  Description of Artificial Sequence Synthetic primer
aggagatataccatggggcatcatcatcatcatcatagcagc

SEQ ID NO: 151 - DNA, 41 bp, Artificial Sequence
  Description of Artificial Sequence Synthetic primer
cagccggatcctcgagttacttgtacagctcgtccatgccg

SEQ ID NO: 152 - DNA, 36 bp, Artificial Sequence
  Description of Artificial Sequence Synthetic primer
gtacaagggcggtaccatggtgagcaagggcgagga

SEQ ID NO: 153 - DNA, 36 bp, Artificial Sequence
  Description of Artificial Sequence Synthetic primer
caccatgctccctcccttgtacagctcgtccatgcc

SEQ ID NO: 154 - DNA, 36 bp, Artificial Sequence
  Description of Artificial Sequence Synthetic primer
ctgagctcactcgagaacgtgtatattaccgcggat

SEQ ID NO: 155 - DNA, 42 bp, Artificial Sequence
  Description of Artificial Sequence Synthetic primer
tcagtcagttggtccggcaggttatattccagtttatgcccc

SEQ ID NO: 156 - DNA, 40 bp, Artificial Sequence
  Description of Artificial Sequence Synthetic primer
ggcataaactggaatataacctgccggaccaactgactga

SEQ ID NO: 157 - DNA, 24 bp, Artificial Sequence
  Description of Artificial Sequence Synthetic primer
cagccggatcaagcttcgaattgc

SEQ ID NO: 158 - protein, 5 aa, Artificial Sequence
  Description of Artificial Sequence Synthetic peptide
KKKRK

SEQ ID NO: 159 - protein, 26 aa, Artificial Sequence
  Description of Artificial Sequence Synthetic peptide
MLRTSSLFTRRVQPSLFRNILRLQST

SEQ ID NO: 160 - protein, 4 aa, Artificial Sequence
  Description of Artificial Sequence Synthetic peptide
KDEL

SEQ ID NO: 161 - protein, 4 aa, Artificial Sequence
  Description of Artificial Sequence Synthetic peptide
  MOD_RES (4)..(4): Any amino acid
CAAX

SEQ ID NO: 162 - protein, 4 aa, Artificial Sequence
  Description of Artificial Sequence Synthetic peptide
  MOD_RES (3)..(4): Any amino acid
CCXX

SEQ ID NO: 163 - DNA, 1437 bp, Artificial Sequence
  Description of Artificial Sequence Synthetic polynucleotide
ttgtccaacgtgtatattaccgcggataaacagaaaaacggcattaaagcgaactttcacgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaat

SEQ ID NO: 164 - protein, 479 aa, Artificial Sequence
  Description of Artificial Sequence Synthetic polypeptide
LSNVYITADKQKNGIKANFHVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFN

SEQ ID NO: 165 - DNA, 1497 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtttaa

SEQ ID NO: 166 - DNA, 2229 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 167 - protein, 743 aa, Artificial Sequence; identical to SEQ ID NO: 68
  Description of Artificial Sequence Synthetic polypeptide

SEQ ID NO: 168 - DNA, 2229 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaatgtcccaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 169 - protein, 743 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKCPNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 170 - protein, 743 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKFPNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 171 - DNA, 2229 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaatttcctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtccagtccaagctgagcaaagaccccaacgagaagcgcgatcacatggtcctgctggagttcgtgaccgccgccgggatcactctcggcatggacgagctgtacaagggtggtaccggtggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 172 - protein, 741 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLENVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 173 - DNA, 2223 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaactcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 174 - protein, 741 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLSNVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 175 - DNA, 2223 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 176 - protein, 741 aa, Artificial Sequence; identical to SEQ ID NO: 65
  Description of Artificial Sequence Synthetic polypeptide

SEQ ID NO: 177 - DNA, 2223 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 178 - protein, 977 aa, Artificial Sequence; identical to SEQ ID NO: 71
  Description of Artificial Sequence Synthetic polypeptide

SEQ ID NO: 179 - DNA, 2931 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 180 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAIAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 181 - DNA, 2931 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgatcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 182 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAFAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKGSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLIWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 183 - DNA, 2931 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaaggtagtaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcatatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 184 - protein, 977 aa, Artificial Sequence
  Description of Artificial Sequence Synthetic polypeptide
MSGAITCSAADLATLLGPNATAAADYICGQLGTVNNKFTDAAFAIDNTYLLFSAYLVFAMQLGFAMLCAGSVRAKNTMNIMLTNVLDAAAGGLFYYLFGYAFAFGGSSEGFIGRHNFALRDFPTPTADYSFFLYQWAIAIAAAGITSGSIAERTQFVAYLIYSSFLTGFVYPVVSHWFWSPDGWASPFRSADDRLFSTGAIDFAGSGVVHMVGGIAGLWGALIEGPRRGRFEKLSNVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNGGRAIALRGHSASLVVLGTFLLWFGWYGFNPGSFTKILVPYNSGSNYGQWSGIGRTAVNTTLSGCTAALTTLFGKRLLSGHWNVTDVCNGLLGGFAAITAGCSVVEPWAAIVCGFMASVVLIGCNKLAELVQYDDPLEAAQLHGGCGAWGLIFVGLFAKEKYLNEVYGATPGRPYGLFMGGGGKLLGAQLVQILVIVGWVSATMGTLFFILKRLNLLRISEQHEMQGMDMTRHGGFAYIYHDNDDESHRVDPGSPFPRSATPPRV

SEQ ID NO: 185 - DNA, 2931 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgatcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcctatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 186 - protein, 977 aa, Artificial Sequence; identical to SEQ ID NO: 66
  Description of Artificial Sequence Synthetic polypeptide

SEQ ID NO: 187 - DNA, 2931 bp, Artificial Sequence
  Description of Artificial Sequence Synthetic polynucleotide
atgtcaggagcaataacatgctctgcggccgatctcgccaccctacttggccccaacgccacggcggcggccgactacatttgcggccaattaggcaccgttaacaacaagttcaccgatgcagccttcgccatagacaacacctacctcctcttctctgcctaccttgtcttcgccatgcagctcggcttcgctatgctttgtgctggttctgttagagccaagaatacgatgaacatcatgcttaccaatgtccttgacgctgcagccggaggactcttctactatctctttggttacgcctttgcctttggaggatcctccgaagggttcattggaagacacaactttgctcttagagactttccgactcccacagctgattactctttcttcctctaccaatgggcgttcgcaatcgcggccgctggaatcacaagtggttcgatcgcagagaggactcagttcgtggcttacttgatatactcttctttcttaaccggatttgtttacccggttgtctctcactggttttggtccccggatggatgggccagtccctttcgttcagcggatgatcgtttgtttagcaccggagccattgactttgctggctccggtgttgttcacatggttggtggcatagcaggtttatggggtgctcttattgaaggtcctcgtcgtggtcggttcgagaaattgtccaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataactttaatggtggtcgcgctattgctctgcgcggccactctgcctcgctagtagtcttaggaaccttcctcatatggtttggatggtatggtttcaaccccggttccttcactaagatactcgttccgtataattctggttccaactacggccaatggagcggaatcggccgtacagcggttaacaccacactctcaggatgcacagcagctctaaccacactctttggtaaacgtctcctatcaggccactggaacgtaacggacgtttgcaacgggttactcggtgggtttgcggccataaccgcaggttgctccgtcgtagagccatgggcagcgattgtgtgcggcttcatggcttctgtcgtccttatcggatgcaacaagctcgcggagcttgtacaatatgatgatccactcgaggcagcccaactacatggagggtgtggcgcgtgggggttgatattcgtaggattgtttgccaaagagaagtatctaaacgaggtttatggcgccaccccgggaaggccatatggactatttatgggcggaggagggaagctgttgggagcacaattggttcaaatacttgtgattgtaggatgggttagtgccacaatgggaacactcttcttcatcctcaaaaggctcaatctgcttaggatctcggagcagcatgaaatgcaagggatggatatgacacgtcacggtggctttgcttatatctaccatgataatgatgatgagtctcatagagtggatcctggatctcctttccctcgatcagctactcctcctcgcgtt

SEQ ID NO: 188 - protein, 413 aa, Artificial Sequence
  Description of Artificial Sequence Synthetic polypeptide
SSRRKWNKTGHAVRAIGRLSSLENVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 189 - DNA, 1242 bp, Artificial Sequence
  Description of Artificial Sequence Synthetic polynucleotide
tcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 190 - protein, 411 aa, Artificial Sequence
  Description of Artificial Sequence Synthetic polypeptide
SSRRKWNKTGHAVRAIGRLSSLENVYITADKQKNGIKANFTVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 191 - DNA, 1236 bp, Artificial Sequence
  Description of Artificial Sequence Synthetic polynucleotide
tcatcacgtcgtaagtggaataagacaggtcacgcagtcagagctataggtcggctgagctcactcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 192 - protein, 411 aa, Artificial Sequence
  Description of Artificial Sequence Synthetic polypeptide
SSRRKWNKTGHAVRAIGRLSSLENVYITADKQKNGIKANFHVRHNVEDGSVQLADHYQQNTPIGDGPVLLPDNHYLSTQTKLSKDPNEKRDHMVLLEFVTAAGITHGMDELYGGTGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 193 - DNA, 1236 bp, Artificial Sequence
  Description of Artificial Sequence Synthetic polynucleotide
tcatcacgtcgtaagtggaataagacaggtcacgcagtcagagctataggtcggctgagctcactcgagaacgtgtatattaccgcggataaacagaaaaacggcattaaagcgaactttcacgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 194 - protein, 649 aa, Artificial Sequence
  Description of Artificial Sequence Synthetic polypeptide
SSRRKWNKTGHAVRAIGRLSSLENVYIKADKQKNGIKANFHIRHNIEDGGVQLAYHYQQNTPIGDGPVLLPDNHYLSVQSKLSKDPNEKRDHMVLLEFVTAAGITLGMDELYKGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 195 - DNA, 1950 bp, Artificial Sequence
  Description of Artificial Sequence Synthetic polynucleotide
tcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 196 - protein, 647 aa, Artificial Sequence
  Description of Artificial Sequence Synthetic polypeptide
SSRRKWNKTGHAVRAIGRLSSLENVYITADKQKNGIKANFTVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 197 - DNA, 1944 bp, Artificial Sequence
  Description of Artificial Sequence Synthetic polynucleotide
tcatcacgtcgtaagtggaataagacaggtcacgcagtcagagctataggtcggctgagctcactcgagaacgtgtatattaccgcggataaacagaaaaacggcattaaagcgaactttaccgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 198 - protein, 647 aa, Artificial Sequence
  Description of Artificial Sequence Synthetic polypeptide
SSRRKWNKTGHAVRAIGRLSSLENVYITADKQKNGIKANFHVRHNVEDGSVQLADHYQQNTPIGDGPVLLPDNHYLSTQTKLSKDPNEKRDHMVLLEFVTAAGITHGMDELYGGTMVSKGEENNMAIIKEFMRFKVRMEGSVNGHEFEIEGEGEGRPYEGFQTVKLKVTKGGPLPFAWDILSPQFTYGSKAYVKHPADIPDYLKLSFPEGFKWERVMNFEDGGVVTVTQDSSLQDGEFIYKVKLRGTNFPSDGPVMQKKTMGMEASSERMYPEDGALKGEDKLRLKLKDGGHYTSEVKTTYKAKKPVQLPGAYIVDIKLDITSHNEDYTIVEQYERAEGRHSTGGMDELYKGGSASQGEELFTGVVPILVELDGDVNGHKFSVRGEGEGDATIGKLTLKFISTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNLPDQLTEEQIAEFKEAFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 199 - DNA, 1944 bp, Artificial Sequence
  Description of Artificial Sequence Synthetic polynucleotide
tcatcacgtcgtaagtggaataagacaggtcacgcagtcagagctataggtcggctgagctcactcgagaacgtgtatattaccgcggataaacagaaaaacggcattaaagcgaactttcacgtgcgccataacgtggaagatggcagcgtgcagctggcggatcattatcagcagaacaccccgattggcgatggcccggtgctgctgccggataaccattatctgagcacccagaccaagctgagcaaagatccgaacgaaaaacgcgatcacatggtgctgctggaatttgtgaccgcagcgggcattacacacggcatggatgaactgtatggcggcaccatggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgttaagctgaaggtgaccaagggtggccccctgcccttcgcctgggacatcttgtcccctcagttcacctacggctccaaggcctacgtgaagcaccccgccgacatccccgactacctcaagctgtccttccccgagggcttcaagtgggagcgcgtgatgaacttcgaggacggcggcgtggtgaccgtgactcaggactcctccctgcaggacggcgagttcatctacaaggtgaagctgcgcggcaccaacttcccctccgacggccccgtaatgcagaagaagaccatgggcatggaggcctcctccgagcggatgtaccccgaggacggcgccctgaagggcgaggacaagctcaggctgaagctgaaggacggcggccactacacctccgaggtcaagaccacctacaaggccaagaagcccgtgcagttgcccggcgcctacatcgtcgacatcaagttggacatcacctcccacaacgaggactacaccatcgtggaacagtacgaacgcgccgagggccgccactccaccggcggcatggacgagctgtacaagggcggcagcgcgagccagggcgaagaactgtttaccggcgtggtgccgattctggtggaactggatggcgatgtgaacggccataaatttagcgtgcgcggcgaaggcgaaggcgatgcgaccattggcaaactgaccctgaaatttatttccaccaccggcaaactaccggtgccgtggccgaccctggtgaccaccttaacctatggcgtgcagtgctttagccgctatccggatcatatgaaacgccatgatttttttaaaagcgcgatgccggaaggctatgtgcaggaacgcaccattagctttaaagatgatggcaaatataaaacccgcgcggtggtgaaatttgaaggcgataccctggtgaaccgcattgaactgaaaggcaccgattttaaagaagatggcaacattctggggcataaactggaatataacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 200 - DNA, 3 bp, Artificial Sequence
  Description of Artificial Sequence Synthetic oligonucleotide
ggt

SEQ ID NO: 201 - protein, 3 aa, Artificial Sequence
  Description of Artificial Sequence Synthetic peptide
GEL

SEQ ID NO: 202 - DNA, 3 bp, Artificial Sequence
  Description of Artificial Sequence Synthetic oligonucleotide
ggt

SEQ ID NO: 203 - protein, 3 aa, Artificial Sequence
  Description of Artificial Sequence Synthetic peptide
FKT

SEQ ID NO: 204 - protein, 3 aa, Artificial Sequence
  Description of Artificial Sequence Synthetic peptide
RHN

SEQ ID NO: 205 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttctcctacggcgtgatggtgttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcatccagtccaagctgagcaaagaccccaacgagaagcgcgatcacatggtcctgctggagttcgtgaccgccgccgggatcactctgggcatggacgagctgtacaagtaa

SEQ ID NO: 206 - protein, 240 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTFSYGVMVFARYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGGVQLADHYQQNTPIGDGPVLLPDNHYLSIQSKLSKDPNEKRDHMVLLEFVTAAGITLGMDELYKF